SEQUENCE LISTING (44 sequences)

SEQ ID NO: 1 - protein, 126 aa, synthetic construct
EVQLLEEVQLVESGGGLVQPGGSLRLSCAASGFTFDDYGMSWVRQAPGKWLEWVSDISWNGGSTYYADSVKGRFTISRDNAENTLYLQMNSLKPDDTAVYYCAKMGEGGWGANDYWGQGTQVTVSS

SEQ ID NO: 2 - protein, 111 aa, synthetic construct
EVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSS

SEQ ID NO: 3 - protein, 126 aa, Homo sapiens
MADEIAKAQVARPGGDTIFGKIIRKEIPAKIIFEDDRSLAFHDISPQAPTHFLVIPKKHISQISVAEDDDESLLGHLMIVGKKSAADLGLNKGYRMEVNEGSDGGQSVYHVHLHVLGGRQMHWPPG

SEQ ID NO: 4 - protein, 6 aa, synthetic construct
GPLGVR

SEQ ID NO: 5 - protein, 17 aa, synthetic construct
GGSGGGPLGVRGSGGEL

SEQ ID NO: 6 - protein, 269 aa, synthetic construct
MADEIAKAQVARPGGDTIFGKIIRKEIPAKIIFEDDRSLAFHDISPQAPTHFLVIPKKHISQISVAEDDDESLLGHLMIVGKKSAADLGLNKGYRMEVNEGSDGGQSVYHVHLHVLGGRQMHWPPGGGSGGGPLGVRGSGGELEVQLLEEVQLVESGGGLVQPGGSLRLSCAASGFTFDDYGMSWVRQAPGKWLEWVSDISWNGGSTYYADSVKGRFTISRDNAENTLYLQMNSLKPDDTAVYYCAKMGEGGWGANDYWGQGTQVTVSS

SEQ ID NO: 7 - protein, 277 aa, synthetic construct
MGHHHHHHMADEIAKAQVARPGGDTIFGKIIRKEIPAKIIFEDDRSLAFHDISPQAPTHFLVIPKKHISQISVAEDDDESLLGHLMIVGKKSAADLGLNKGYRMEVNEGSDGGQSVYHVHLHVLGGRQMHWPPGGGSGGGPLGVRGSGGELEVQLLEEVQLVESGGGLVQPGGSLRLSCAASGFTFDDYGMSWVRQAPGKWLEWVSDISWNGGSTYYADSVKGRFTISRDNAENTLYLQMNSLKPDDTAVYYCAKMGEGGWGANDYWGQGTQVTVSS

SEQ ID NO: 8 - protein, 254 aa, synthetic construct
MADEIAKAQVARPGGDTIFGKIIRKEIPAKIIFEDDRSLAFHDISPQAPTHFLVIPKKHISQISVAEDDDESLLGHLMIVGKKSAADLGLNKGYRMEVNEGSDGGQSVYHVHLHVLGGRQMHWPPGGGSGGGPLGVRGSGGELEVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSS

SEQ ID NO: 9 - protein, 262 aa, synthetic construct
MGHHHHHHMADEIAKAQVARPGGDTIFGKIIRKEIPAKIIFEDDRSLAFHDISPQAPTHFLVIPKKHISQISVAEDDDESLLGHLMIVGKKSAADLGLNKGYRMEVNEGSDGGQSVYHVHLHVLGGRQMHWPPGGGSGGGPLGVRGSGGELEVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSS

SEQ ID NO: 10 - protein, 27 aa, synthetic construct
QDGNEEMGGITQTPYKVSISGTTVILT

SEQ ID NO: 11 - protein, 170 aa, synthetic construct
QDGNEEMGGITQTPYKVSISGTTVILTGGSGGGPLGVRGSGGELEVQLLEEVQLVESGGGLVQPGGSLRLSCAASGFTFDDYGMSWVRQAPGKWLEWVSDISWNGGSTYYADSVKGRFTISRDNAENTLYLQMNSLKPDDTAVYYCAKMGEGGWGANDYWGQGTQVTVSS

SEQ ID NO: 12 - protein, 178 aa, synthetic construct
MGHHHHHHQDGNEEMGGITQTPYKVSISGTTVILTGGSGGGPLGVRGSGGELEVQLLEEVQLVESGGGLVQPGGSLRLSCAASGFTFDDYGMSWVRQAPGKWLEWVSDISWNGGSTYYADSVKGRFTISRDNAENTLYLQMNSLKPDDTAVYYCAKMGEGGWGANDYWGQGTQVTVSS

SEQ ID NO: 13 - protein, 159 aa, synthetic construct
GISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERR

SEQ ID NO: 14 - protein, 319 aa, synthetic construct
GISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERRGGISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERR

SEQ ID NO: 15 - protein, 5 aa, synthetic construct
GGGGS

SEQ ID NO: 16 - protein, 10 aa, synthetic construct
GGGGSGGGGS

SEQ ID NO: 17 - protein, 5 aa, synthetic construct
GGSGG

SEQ ID NO: 18 - protein, 9 aa, synthetic construct
GGSGGGSGG

SEQ ID NO: 19 - protein, 15 aa, synthetic construct
GGGASGGGGSGGGGS

SEQ ID NO: 20 - protein, 6 aa, synthetic construct
HHHHHH

SEQ ID NO: 21 - protein, 624 aa, synthetic construct
MGHHHHHHMADEIAKAQVARPGGDTIFGKIIRKEIPAKIIFEDDRSLAFHDISPQAPTHFLVIPKKHISQISVAEDDDESLLGHLMIVGKKSAADLGLNKGYRMEVNEGSDGGQSVYHVHLHVLGGRQMHWPPGGGSGGGPLGVRGSGGELEVQLLEEVQLVESGGGLVQPGGSLRLSCAASGFTFDDYGMSWVRQAPGKWLEWVSDISWNGGSTYYADSVKGRFTISRDNAENTLYLQMNSLKPDDTAVYYCAKMGEGGWGANDYWGQGTQVTVSSGGGASGGGGSGGGGSGISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERRGGISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERRGGGLKDYKDDDDK

SEQ ID NO: 22 - protein, 481 aa, synthetic construct
MGHHHHHHEVQLLEEVQLVESGGGLVQPGGSLRLSCAASGFTFDDYGMSWVRQAPGKWLEWVSDISWNGGSTYYADSVKGRFTISRDNAENTLYLQMNSLKPDDTAVYYCAKMGEGGWGANDYWGQGTQVTVSSGGGASGGGGSGGGGSGISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERRGGISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERRGGGLKDYKDDDDK

SEQ ID NO: 23 - protein, 609 aa, synthetic construct
MGHHHHHHMADEIAKAQVARPGGDTIFGKIIRKEIPAKIIFEDDRSLAFHDISPQAPTHFLVIPKKHISQISVAEDDDESLLGHLMIVGKKSAADLGLNKGYRMEVNEGSDGGQSVYHVHLHVLGGRQMHWPPGGGSGGGPLGVRGSGGELEVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSSGGGASGGGGSGGGGSGISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERRGGISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERRGGGLKDYKDDDDK

SEQ ID NO: 24 - protein, 475 aa, synthetic construct
MGHHHHHHGGSGGGSGGEVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSSGGGASGGGGSGGGGSGISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERRGGISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERRGGGLKDYKDDDDK

SEQ ID NO: 25 - protein, 525 aa, synthetic construct
MGHHHHHHQDGNEEMGGITQTPYKVSISGTTVILTGGSGGGPLGVRGSGGELEVQLLEEVQLVESGGGLVQPGGSLRLSCAASGFTFDDYGMSWVRQAPGKWLEWVSDISWNGGSTYYADSVKGRFTISRDNAENTLYLQMNSLKPDDTAVYYCAKMGEGGWGANDYWGQGTQVTVSSGGGASGGGGSGGGGSGISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERRGGISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERRGGGLKDYKDDDDK

SEQ ID NO: 26 - DNA, 1875 bp, synthetic construct
  source 1..1875: mol_type = other DNA
atgggccaccatcaccatcatcatatggcagatgagattgccaaggctcaggtcgctcggcctggtggcgacacgatctttgggaagatcatccgcaaggaaataccagccaaaatcatttttgaggatgaccggagccttgctttccatgacatttcccctcaagcaccaacacattttctggtgatacccaagaaacatatatcccagatttctgtggcagaagatgatgatgaaagtcttcttggacacttaatgattgttggcaagaaaagcgctgctgatctgggcctgaataagggttatcgaatggaagtgaatgaaggttcagatggtggacagtctgtctatcacgttcatctccatgttcttggaggtcggcaaatgcattggcctcctggtggaggttcaggtggtggaccgctgggagtgcgcggcagcggaggcgagctcgaggtgcagctgctcgaggaggtgcagctggtggagtctgggggaggcttggtgcagcctggggggtctctgagactctcctgtgcagcctctggattcacttttgatgattatggcatgagctgggtccgacaggctccagggaagtggctggagtgggtctcagatattagctggaatggtggtagcacatactatgcagactccgtgaagggccggttcaccatctccagagacaacgccgagaacacgctgtatctgcaaatgaacagcctgaaacctgacgacacggccgtgtattactgtgcaaaaatgggtgaagggggatggggtgcaaatgactactggggccaggggacccaggtcaccgtctcctccggaggcggagctagcggtggaggcggctcagggggaggtggatccggaatctcgttgattgcggcattagcggtcgaccgcgttatcggaatggaaaacgcgatgccctggaatttacctgctgaccttgcttggttcaagcgtaacactttaaacaagccggtgattatgggacgccacacgtgggaatccatcggccgccctctgccgggacgtaagaacatcattctttcaagccaaccaggaaccgacgatcgcgtgacgtgggtcaagagtgtcgacgaagcaatcgcggccgcgggagacgtcccggaaatcatggtcatcggaggaggacgtgtctatgagcagtttttgcctaaggcgcagaagctgtacttaacccatatcgacgcagaggtggagggcgacacacacttccccgattacgagcccgatgattgggagtcagtgttctcagaatttcacgacgcggatgcgcagaactctcactcttatagtttcgagattttggagcgccgcggtggaattagtcttatcgctgcgttggcagtcgatcgcgtaatcggtatggagaatgctatgccttggaaccttcccgcagacttggcctggttcaaacgcaatactttaaataaacctgtgattatgggccgtcatacttgggagtcgatcgggcgtcctttgcccggacgcaagaatatcattttgagttcccaaccgggcaccgatgatcgtgttacgtgggttaagagtgtggacgaagctatcgccgctgctggggacgtacccgaaattatggttattgggggtggacgcgtatatgagcaatttctgccgaaagcccaaaaactttatcttacccacattgatgccgaagtggaaggcgatacgcatttcccggactatgagccggatgattgggaatcagtgtttagcgagtttcacgatgcagacgctcagaacagtcattcatactcgtttgagattttagagcgccgtggaggtggccttaaggattacaaggacgacgatgacaagtaa

SEQ ID NO: 27 - DNA, 1446 bp, synthetic construct
  source 1..1446: mol_type = other DNA
atgggccaccatcaccatcatcatgaggtgcagctgctcgaggaggtgcagctggtggagtctgggggaggcttggtgcagcctggggggtctctgagactctcctgtgcagcctctggattcacttttgatgattatggcatgagctgggtccgacaggctccagggaagtggctggagtgggtctcagatattagctggaatggtggtagcacatactatgcagactccgtgaagggccggttcaccatctccagagacaacgccgagaacacgctgtatctgcaaatgaacagcctgaaacctgacgacacggccgtgtattactgtgcaaaaatgggtgaagggggatggggtgcaaatgactactggggccaggggacccaggtcaccgtctcctccggaggcggagctagcggtggaggcggctcagggggaggtggatccggaatctcgttgattgcggcattagcggtcgaccgcgttatcggaatggaaaacgcgatgccctggaatttacctgctgaccttgcttggttcaagcgtaacactttaaacaagccggtgattatgggacgccacacgtgggaatccatcggccgccctctgccgggacgtaagaacatcattctttcaagccaaccaggaaccgacgatcgcgtgacgtgggtcaagagtgtcgacgaagcaatcgcggccgcgggagacgtcccggaaatcatggtcatcggaggaggacgtgtctatgagcagtttttgcctaaggcgcagaagctgtacttaacccatatcgacgcagaggtggagggcgacacacacttccccgattacgagcccgatgattgggagtcagtgttctcagaatttcacgacgcggatgcgcagaactctcactcttatagtttcgagattttggagcgccgcggtggaattagtcttatcgctgcgttggcagtcgatcgcgtaatcggtatggagaatgctatgccttggaaccttcccgcagacttggcctggttcaaacgcaatactttaaataaacctgtgattatgggccgtcatacttgggagtcgatcgggcgtcctttgcccggacgcaagaatatcattttgagttcccaaccgggcaccgatgatcgtgttacgtgggttaagagtgtggacgaagctatcgccgctgctggggacgtacccgaaattatggttattgggggtggacgcgtatatgagcaatttctgccgaaagcccaaaaactttatcttacccacattgatgccgaagtggaaggcgatacgcatttcccggactatgagccggatgattgggaatcagtgtttagcgagtttcacgatgcagacgctcagaacagtcattcatactcgtttgagattttagagcgccgtggaggtggccttaaggattacaaggacgacgatgacaagtaa

SEQ ID NO: 28 - DNA, 1830 bp, synthetic construct
  source 1..1830: mol_type = other DNA
atgggccaccatcaccatcatcatatggcagatgagattgccaaggctcaggtcgctcggcctggtggcgacacgatctttgggaagatcatccgcaaggaaataccagccaaaatcatttttgaggatgaccggagccttgctttccatgacatttcccctcaagcaccaacacattttctggtgatacccaagaaacatatatcccagatttctgtggcagaagatgatgatgaaagtcttcttggacacttaatgattgttggcaagaaaagcgctgctgatctgggcctgaataagggttatcgaatggaagtgaatgaaggttcagatggtggacagtctgtctatcacgttcatctccatgttcttggaggtcggcaaatgcattggcctcctggtggaggttcaggtggtggaccgctgggagtgcgcggtagcggaggcgagctcgaagtgcaattagtagagagtggcggggggcttgttcagcccggaggtagcttgactctttcctgcgcggccagccgttttatgatttccgagtattctatgcactgggtgcgtcaggcgcccggcaagggactggaatgggtcagtacgattaatcccgccggtacgaccgattatgcggagagcgtaaaaggccgtttcactatctctcgcgataacgccaaaaataccttatatttgcaaatgaattcccttaaaccagaagatacggctgtctactattgcgacggctacggataccgtggccaagggacccaagtcacggtttcatcaggaggcggagctagcggtggaggcggctcagggggaggtggatccggaatctcgttgattgcggcattagcggtcgaccgcgttatcggaatggaaaacgcgatgccctggaatttacctgctgaccttgcttggttcaagcgtaacactttaaacaagccggtgattatgggacgccacacgtgggaatccatcggccgccctctgccgggacgtaagaacatcattctttcaagccaaccaggaaccgacgatcgcgtgacgtgggtcaagagtgtcgacgaagcaatcgcggccgcgggagacgtcccggaaatcatggtcatcggaggaggacgtgtctatgagcagtttttgcctaaggcgcagaagctgtacttaacccatatcgacgcagaggtggagggcgacacacacttccccgattacgagcccgatgattgggagtcagtgttctcagaatttcacgacgcggatgcgcagaactctcactcttatagtttcgagattttggagcgccgcggtggaattagtcttatcgctgcgttggcagtcgatcgcgtaatcggtatggagaatgctatgccttggaaccttcccgcagacttggcctggttcaaacgcaatactttaaataaacctgtgattatgggccgtcatacttgggagtcgatcgggcgtcctttgcccggacgcaagaatatcattttgagttcccaaccgggcaccgatgatcgtgttacgtgggttaagagtgtggacgaagctatcgccgctgctggggacgtacccgaaattatggttattgggggtggacgcgtatatgagcaatttctgccgaaagcccaaaaactttatcttacccacattgatgccgaagtggaaggcgatacgcatttcccggactatgagccggatgattgggaatcagtgtttagcgagtttcacgatgcagacgctcagaacagtcattcatactcgtttgagattttagagcgccgtggaggtggccttaaggattacaaggacgacgatgacaagtaa

SEQ ID NO: 29 - DNA, 1428 bp, synthetic construct
  source 1..1428: mol_type = other DNA
atgggccaccatcaccatcatcatggcggtagcggaggcggttcaggtggtgaagtgcaattagtagagagtggcggggggcttgttcagcccggaggtagcttgactctttcctgcgcggccagccgttttatgatttccgagtattctatgcactgggtgcgtcaggcgcccggcaagggactggaatgggtcagtacgattaatcccgccggtacgaccgattatgcggagagcgtaaaaggccgtttcactatctctcgcgataacgccaaaaataccttatatttgcaaatgaattcccttaaaccagaagatacggctgtctactattgcgacggctacggataccgtggccaagggacccaagtcacggtttcatcaggaggcggagctagcggtggaggcggctcagggggaggtggatccggaatctcgttgattgcggcattagcggtcgaccgcgttatcggaatggaaaacgcgatgccctggaatttacctgctgaccttgcttggttcaagcgtaacactttaaacaagccggtgattatgggacgccacacgtgggaatccatcggccgccctctgccgggacgtaagaacatcattctttcaagccaaccaggaaccgacgatcgcgtgacgtgggtcaagagtgtcgacgaagcaatcgcggccgcgggagacgtcccggaaatcatggtcatcggaggaggacgtgtctatgagcagtttttgcctaaggcgcagaagctgtacttaacccatatcgacgcagaggtggagggcgacacacacttccccgattacgagcccgatgattgggagtcagtgttctcagaatttcacgacgcggatgcgcagaactctcactcttatagtttcgagattttggagcgccgcggtggaattagtcttatcgctgcgttggcagtcgatcgcgtaatcggtatggagaatgctatgccttggaaccttcccgcagacttggcctggttcaaacgcaatactttaaataaacctgtgattatgggccgtcatacttgggagtcgatcgggcgtcctttgcccggacgcaagaatatcattttgagttcccaaccgggcaccgatgatcgtgttacgtgggttaagagtgtggacgaagctatcgccgctgctggggacgtacccgaaattatggttattgggggtggacgcgtatatgagcaatttctgccgaaagcccaaaaactttatcttacccacattgatgccgaagtggaaggcgatacgcatttcccggactatgagccggatgattgggaatcagtgtttagcgagtttcacgatgcagacgctcagaacagtcattcatactcgtttgagattttagagcgccgtggaggtggccttaaggattacaaggacgacgatgacaagtaa

SEQ ID NO: 30 - DNA, 1578 bp, synthetic construct
  source 1..1578: mol_type = other DNA
atgggccaccatcaccatcatcatcaggatggcaacgaagaaatgggcggcattacccagaccccgtataaagtgagcattagcggcaccaccgtgattctgaccggaggttcaggtggtggaccgctgggagtgcgcggcagcggaggcgagctcgaggtgcagctgctcgaggaggtgcagctggtggagtctgggggaggcttggtgcagcctggggggtctctgagactctcctgtgcagcctctggattcacttttgatgattatggcatgagctgggtccgacaggctccagggaagtggctggagtgggtctcagatattagctggaatggtggtagcacatactatgcagactccgtgaagggccggttcaccatctccagagacaacgccgagaacacgctgtatctgcaaatgaacagcctgaaacctgacgacacggccgtgtattactgtgcaaaaatgggtgaagggggatggggtgcaaatgactactggggccaggggacccaggtcaccgtctcctccggaggcggagctagcggtggaggcggctcagggggaggtggatccggaatctcgttgattgcggcattagcggtcgaccgcgttatcggaatggaaaacgcgatgccctggaatttacctgctgaccttgcttggttcaagcgtaacactttaaacaagccggtgattatgggacgccacacgtgggaatccatcggccgccctctgccgggacgtaagaacatcattctttcaagccaaccaggaaccgacgatcgcgtgacgtgggtcaagagtgtcgacgaagcaatcgcggccgcgggagacgtcccggaaatcatggtcatcggaggaggacgtgtctatgagcagtttttgcctaaggcgcagaagctgtacttaacccatatcgacgcagaggtggagggcgacacacacttccccgattacgagcccgatgattgggagtcagtgttctcagaatttcacgacgcggatgcgcagaactctcactcttatagtttcgagattttggagcgccgcggtggaattagtcttatcgctgcgttggcagtcgatcgcgtaatcggtatggagaatgctatgccttggaaccttcccgcagacttggcctggttcaaacgcaatactttaaataaacctgtgattatgggccgtcatacttgggagtcgatcgggcgtcctttgcccggacgcaagaatatcattttgagttcccaaccgggcaccgatgatcgtgttacgtgggttaagagtgtggacgaagctatcgccgctgctggggacgtacccgaaattatggttattgggggtggacgcgtatatgagcaatttctgccgaaagcccaaaaactttatcttacccacattgatgccgaagtggaaggcgatacgcatttcccggactatgagccggatgattgggaatcagtgtttagcgagtttcacgatgcagacgctcagaacagtcattcatactcgtttgagattttagagcgccgtggaggtggccttaaggattacaaggacgacgatgacaagtaa

SEQ ID NO: 31 - protein, 129 aa, Homo sapiens
SNAMADEIAKAQVARPGGDTIFGKIIRKEIPAKIIFEDDRSLAFHDISPQAPTHFLVIPKKHISQISVAEDDDESLLGHLMIVGKKSAADLGLNKGYRMEVNEGSDGGQSVYHVHLHVLGGRQMHWPPG

SEQ ID NO: 32 - protein, 601 aa, synthetic construct
MGHHHHHHGGSGGGSGGGSGGMISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERRGMISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERRGGSGGGSGGGSGGDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRGGGGSGGGGSGGGGSGGREVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSS

SEQ ID NO: 33 - DNA, 1803 bp, synthetic construct
  source 1..1803: mol_type = other DNA
atgggacaccatcaccatcaccacggtggttcaggtggtggttcaggaggtggatctggaggtatgatcagtctgattgcggcgttagcggtagatcgcgtgattggtatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgggtacggacgatcgcgtaacgtgggtgaagtcggtggatgaagccatcgcggcggctggtgacgtaccagaaatcatggtgattggcggcggtcgcgtttatgaacagttcttgccaaaagcgcaaaaactgtatctgacgcatatcgacgcagaagtggaaggcgacacccatttcccggattacgagccggatgactgggaatcggtattcagtgaattccacgatgctgatgcgcagaactctcacagctatagctttgagattctggagcggcggggcatgatctctctgatcgcggctcttgctgttgatcgcgttatcggcatggagaatgcaatgccttggaacttgccagcggaccttgcttggttcaagcgtaatacattgaataaacctgtaatcatgggtcgccatacgtgggagtcgatcggccgccctctgcccggccgcaaaaacatcattctgagctctcaaccaggtactgatgaccgtgttacgtgggttaaaagtgtagacgaagccattgcagctgcgggtgatgtacccgagattatggtaatcggaggggggcgtgtatacgaacagttcttgcccaaggcgcaaaagttatatttgacgcatattgatgccgaagtcgaaggcgatacacattttccggattacgagcctgatgactgggaatcggttttttccgagtttcatgacgcggatgcccaaaactctcacagttattcttttgagattcttgaacgtcgcgggggcagtggaggtggcagtggaggtgggtctgggggcgacattcagatgacgcagacgacatcgagcttatccgcaagcctgggggatcgcgttacgatctcttgtcgtgcatcccaagacatccgcaactacttaaactggtatcaacaaaaaccggatggaaccgtgaaattgcttatctactatacttcgcgcctgcactcgggagtaccctcaaagtttagcggctccgggagtggtacagactatagtcttaccatttccaatcttgaacaggaagacattgcaacgtacttttgccaacaagggaatactcttccttggacttttgcgggagggacgaaacttgagattaaacgcggcgggggaggatcagggggtgggggctcaggaggcggaggttccggcgggcgcgaggtacaattacagcagtccgggccggagcttgttaagccgggagcgagcatgaaaatttcatgtaaggcaagtggctatagcttcactggttataccatgaactgggttaagcaatcgcacggaaagaatctggagtggatgggtttgattaacccctataagggggtttcgacttacaatcaaaagtttaaagacaaagccacactgactgttgataagagttcttcgaccgcatacatggaattgctgtcactgaccagtgaggattcggcggtgtactattgtgcacgcagcggttattatggagattccgactggtacttcgatgtctggggagctggtacaacggtcaccgttagttca

SEQ ID NO: 34 - protein, 13 aa, synthetic construct
GGSGGGSGGGSGG

SEQ ID NO: 35 - protein, 248 aa, synthetic construct
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRGGGGSGGGGSGGGGSGGREVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSS

SEQ ID NO: 36 - protein, 159 aa, synthetic construct
MISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERR

SEQ ID NO: 37 - protein, 319 aa, synthetic construct
MISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERRGMISLIAALAVDRVIGMENAMPWNLPADLAWFKRNTLNKPVIMGRHTWESIGRPLPGRKNIILSSQPGTDDRVTWVKSVDEAIAAAGDVPEIMVIGGGRVYEQFLPKAQKLYLTHIDAEVEGDTHFPDYEPDDWESVFSEFHDADAQNSHSYSFEILERR

SEQ ID NO: 38 - protein, 5 aa, synthetic construct
DYGMS

SEQ ID NO: 39 - protein, 17 aa, synthetic construct
DISWNGGSTYYADSVKG

SEQ ID NO: 40 - protein, 11 aa, synthetic construct
MGEGGWGANDY

SEQ ID NO: 41 - protein, 4 aa, synthetic construct
GGGS

SEQ ID NO: 42 - protein, 6 aa, synthetic construct
GGSGGS

SEQ ID NO: 43 - protein, 6 aa, synthetic construct
GSSGSS

SEQ ID NO: 44 - protein, 6 aa, synthetic construct
GLPGVR

(19) United States
(12) Patent Application Publication
(10) Pub. No.: US 2023/0406927 A1
(43) Pub. Date: Dec. 21, 2023

(54) MASKED SINGLE-DOMAIN ANTIBODIES AND METHODS THEREOF

(71) Applicant: Regents of the University of Minnesota, Minneapolis, MN (US)

(72) Inventors: Carston R. Wagner, Minneapolis, MN (US); Abhishek Kulkarni, Minneapolis, MN (US)

(73) Assignee: Regents of the University of Minnesota, Minneapolis, MN (US)

(21) Appl. No.: 18/141,728

(22) Filed: May 1, 2023

Related U.S. Application Data
(60) Provisional application No. 63/336,932, filed on Apr. 29, 2022.

(51) Int. Cl. C07K 16/28; A61P 35/00; C07K 16/30

(52) U.S. Cl. CPC C07K 16/2809; A61P 35/00; C07K 16/3076; C07K 2317/569; C07K 2317/60

(57) ABSTRACT

Certain embodiments of the invention provide a masked single-domain antibody as described herein, as well as methods of use thereof. Certain embodiments of the invention provide a masked CSAN as described herein, as well as methods of use thereof.

### CROSS-REFERENCE TO RELATED APPLICATION

This application claims priority to U.S. Provisional Application No. 63/336,932 that was filed on Apr. 29, 2022. The entire content of the application referenced above is hereby incorporated by reference herein.

### GOVERNMENT FUNDING

This invention was made with government support under CA185627 and CA247681 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Jul. 31, 2023, is named 09531_558US1_SL.xml and is 55,309 bytes in size.

### BACKGROUND OF THE INVENTION

Tumor-associated antigen targeting relies on differential antigen expression between cancer and healthy cells. However, many current cancer immuno-therapeutics exhibit “on-target, off-tumor” toxicity. Accordingly, methods (e.g., new cancer immunotherapy methods) and compositions are needed.

### SUMMARY OF THE INVENTION

Certain embodiments of the invention provide a recombinant protein, comprising a masking polypeptide linked to a single-domain antibody (sdAb), or an antigen binding fragment thereof, via a protease-sensitive polypeptide linker, wherein the binding affinity of the sdAb, or the antigen binding fragment thereof, to its target antigen is reduced by the masking polypeptide.

Certain embodiments of the invention provide a polynucleotide comprising a nucleic acid sequence encoding a recombinant protein described herein.

Certain embodiments of the invention provide an assembly, such as a chemically self-assembled nanoring (CSAN), comprising a recombinant protein described herein.

Certain embodiments of the invention provide a pharmaceutical composition comprising a recombinant protein or a CSAN described herein, and a pharmaceutically acceptable excipient.

Certain embodiments of the invention provide a method for treating or preventing cancer in an animal (e.g., a mammal such as human), comprising administering a therapeutically effective amount of a recombinant protein or a CSAN described herein to the animal.

Certain embodiments provide a masked single-domain antibody (sdAb; also referenced herein as a nanobody), or an antigen binding fragment thereof, as described herein.

Thus, certain embodiments provide a polypeptide or protein as described herein. For example, certain embodiments provide a protein or polypeptide comprising an amino acid sequence having at least about 70%, 75%, 80%, 85%, 90%, 95%, 96%, 97%, 98%, 99% or 100% sequence identity to a sequence described herein.

Certain embodiments provide a nucleic acid as provided herein. For example, certain embodiments provide a nucleic acid comprising a sequence having at least about 70%, 75%, 80%, 85%, 90%, 95%, 96%, 97%, 98%, 99% or 100% sequence identity to a sequence described herein. Certain embodiments also provide expression cassettes and vectors comprising such nucleic acids.

Certain embodiments provide a protein comprising a human histidine nucleotide triad binding protein 1 (hHINT1) polypeptide linked through a polypeptide linker to a sdAb, or an antigen binding fragment thereof. In certain embodiments, the hHINT1 polypeptide comprises an amino acid sequence having at least about 70%, 75%, 80%, 85%, 90%, 95%, 96%, 97%, 98%, 99% or 100% sequence identity to a hHINT1 sequence described herein.

Certain embodiments also provide a protein comprising a human CD3ε derived peptide linked through a polypeptide linker to a sdAb, or an antigen binding fragment thereof. In certain embodiments, the human CD3ε derived peptide comprises an amino acid sequence having at least about 70%, 75%, 80%, 85%, 90%, 95%, 96%, 97%, 98%, 99% or 100% sequence identity to a human CD3ε peptide sequence described herein.

In certain embodiments, the linker is a flexible polypeptide linker. In certain embodiments, the linker is cleavable polypeptide linker that is capable of being cleaved by a proteolytic enzyme expressed within a solid tumor. In certain embodiments, the cleavable polypeptide linker comprises a polypeptide sequence disclosed herein (e.g., MMP-2 substrate sequence GPLGVR (SEQ ID NO: 4)). In certain embodiments, the proteolytic enzyme is MMP-2.

In certain embodiments, the sdAb is anti-CD3 sdAb, or an antigen binding fragment thereof.

Certain embodiments provide a method as described herein for preparing a masked sdAb.

Certain embodiments provide an assembly (e.g., CSAN or conjugate thereof) comprising a protein or polypeptide as described herein.

Certain embodiments of the invention provide a pharmaceutical composition comprising a protein, polypeptide, assembly, or conjugate thereof described herein and a pharmaceutically acceptable excipient.

Certain embodiments of the invention provide a method comprising contacting a cell with a protein, polypeptide, assembly, or conjugate thereof as described herein. In certain embodiments, the cell is contacted in vitro. In certain embodiments, the cell is contacted in vivo. In certain embodiments, the cell is an immune cell (e.g., T cell). In certain embodiments, the cell is a tumor cell.

Certain embodiments of the invention provide a method for treating or preventing cancer in an animal (e.g., a human) comprising administering a therapeutically effective amount of a protein, polypeptide, assembly, or conjugate thereof described herein to the animal.

The invention also provides a protein, polypeptide, or assembly, or a conjugate described herein for use in medical therapy.

The invention also provides a protein, polypeptide, or assembly, or a conjugate thereof described herein for the prophylactic or therapeutic treatment of cancer.

The invention also provides the use of a protein, polypeptide, or assembly, or a conjugate thereof described herein to prepare a medicament for treating cancer in an animal (e.g., a mammal such as a human).

### BRIEF DESCRIPTION OF THE FIGURES

FIG. 1. Redirection of T cells by EpCAM-targeting bispecific CSANs.

FIG. 2. Potential “on-target, off-tumor” toxicity exhibited by bispecific CSANs.

FIG. 3. Mechanism of action of Pro-CSANs. The Pro-CSANs bind to tumor cells first, followed by unmasking of the anti-CD3 targeting element via MMP-2, leading to engagement and activation of T cells specifically in the TME.

FIGS. 4A-4B. Genetic masking of the anti-CD3 nanobody. FIG. 4A: Crystal structure of a generic nanobody (PDB: 6JB2); CDR=complementarity determining regions. FIG. 4B: Architecture of the steric and affinity masked anti-CD3 DHFR2 fusion proteins.

FIG. 5. Protein production protocol. LB=Luria Broth; Kan=Kanamycin; IMAC=Immobilized metal affinity chromatography.

FIG. 6. SEC analysis of unmasked or masked fusion proteins. SEC analysis (upper panel graph) showed that anti-CD3-1DD monomer as a single peak. Monomeric proteins were incubated with a 3-fold excess of Bis-MTX for 1 hr at room temperature. Upon addition of bisMTX (chemical dimerizer), the protein trace shifts to the left indicating the formation of protein nanorings. Reduction and/or disappearance of the monomer trace indicated nanoring formation. SEC analysis (lower panel graph) also showed that upon addition of bisMTX (chemical dimerizer), hHINT1-anti-CD3-1DD monomer protein can also oligomerize to form nanorings (left-ward shift of the monomer peak into nanoring peak).

FIG. 7. Results of SDS-PAGE analysis demonstrating the unmasking of the fusion proteins. Reactions ran at room temperature.

FIGS. 8A-8E. Flow cytometry histograms depicting binding of proteins to T cells. (FIG. 8A-FIG. 8E) Qualitative binding analysis of masked and unmasked-αCD3-1DD proteins. (FIG. 8D and FIG. 8E) Nanorings were cleaved with 10 μg/mL recombinant MMP-2 for 3 hours at 37° C. followed by incubation with T cells. Tables indicate median fluorescence intensity values; msGFP2=monomeric super-folder green fluorescent protein. For FIGS. 8A-8E, the order of flow cytometry histograms (from top to bottom) matches the order of subset name in the table (from top to bottom).

FIG. 9. Design of the anti-CD3 nanobody-1DD fusion protein. As seen from the crystal structure of a nanobody (PDB: 6JB2), the CDRs are located close to the N-terminus; therefore, the N-terminus of this nanobody was further extended to include a protease-sensitive linker and hHINT1.

FIG. 10. Exemplary designs of masked fusion proteins, such as HINT1 masked-αCD3-1DD fusion protein (upper panel graph) and HINT1 masked-αPSMA-1DD fusion protein (lower panel graph).

FIG. 11. αCD3-1DD and HINT1-αCD3-1DD proteins were successfully cloned and purified.

FIGS. 12A-12C. (FIG. 12A) Soluble protein production and purification in E coli. (FIG. 12B) In vitro MMP-2 cleavage assay. (FIG. 12C) Binding analysis using flow cytometry.

FIG. 13. As seen through the above time-dependent SDS-PAGE analysis, hHINT1-anti-CD3-1DD was almost completely digested by MMP-2 within 24 hours, resulting in the removal of hHINT1 mask and formation of anti-CD3-1DD protein.

FIG. 14. HINT1 mask was removed by MMP-2 in vitro.

FIG. 15. Determination of the apparent affinity of the fusion proteins using flow cytometry. Anti-CD3-1DD fusion protein showed an apparent K_{D} value of 100 nM, whereas the hHINT1-anti-CD3-1DD fusion protein showed an apparent K_{D} of greater than 4000 nM. Therefore, the hHINT1 mask reduced the binding affinity of the anti-CD3 nanobody by 40-fold. Data representative of 1 experiment using T cells isolated from healthy donor blood.

FIG. 16. hHINT1-anti-PSMA-1DD and anti-CD3 scFv-1DD proteins can self-assemble to form masked CSANs or protein nanorings. These nanorings label the surface of T cells that can only crosslink with PSMA expressing prostate cancer cells owing to removal of hHINT1 mask via upregulated MMP-2 activity.

FIG. 17. SEC analysis of unmasked or masked fusion proteins. SEC analysis (upper panel graph) showed that anti-PSMA-1DD monomer as a single peak. Upon addition of bisMTX (chemical dimerizer), the protein trace shifts to the left indicating the formation of protein nanorings. SEC analysis (lower panel graph) also showed that upon addition of bisMTX (chemical dimerizer), hHINT1-anti-PSMA-1DD monomer protein can also oligomerize to form nanorings (left-ward shift of the monomer peak into nanoring peak).

FIG. 18. SDS-PAGE analysis. hHINT1 mask was completely removed by MMP-2 within 24 hours. Similar observations to the hHINT1-anti-CD3-1DD protein indicating the success of the overall fusion protein design strategy.

FIG. 19. Anti-PSMA/anti-CD3 scFv labeled T cells kill PSMA+LNCaP cells. Anti-PSMA/anti-CD3 scFv nanorings efficiently lysed tumor cells over the period of 72 hrs. Anti-CD3 scFv rings (monospecific, do not possess the PSMA-targeting nanobody) showed comparatively lesser tumor cell lysis at 72 hrs. Additionally, the masked protein, hHINT1-anti-PSMA/anti-CD3 scFv showed a reduction in the target cell lysis (approximately 33% reduction) when compared to the anti-PSMA/anti-CD3 scFv treatment.

### DETAILED DESCRIPTION

Described herein are masked single-domain antibodies (sdAbs; also referenced herein as nanobodies), which are capable of being unmasked via cleavage of a masking polypeptide. For example, described herein is the development of an approach that allows the design of masked cancer antigen binding sdAbs that are able to be unmasked by tumor produced protease(s). The masked sdAbs can then act to deliver a drug payload or target immune cells to the tumor, while avoiding targeting to potential antigen expressing normal tissues.

The masked sdAbs or pro-sdAbs incorporate a unique masking group that can be applied to any sdAb. As described in more detail below, in one embodiment, the design involves the preparation of a monomeric version of hHINT1 linked through a flexible soluble linker incorporating a MMP (protease produced by in the tumor microenvironment) proteolytic site to the sdAb, such that the close proximity to the binding site on the sdAb sterically blocks it from binding its target antigen. Once the hHINT1 is removed by the action of tumor expressed protease (e.g., MMP), the sdAb is free to bind the tumor cells and allow for potential immune cell targeting or drug delivery.

In particular, the technology described herein enables the construction of masked sdAbs that can be used for, e.g., cancer immunotherapy. SdAbs are particularly useful as an alternative stratedy due to their smaller size (half the size of a scFv), easier ability to be produced and designed recombinantly and low immunogenicity.

The terms “nanobody” and “single-domain antibody” are used interchangeably herein. As used herein, the term “nanobody” or “single-domain antibody” refers to a single monomeric variable antibody domain comprising three complementarity-determining regions (CDRs including CDR1, CDR2, CDR3) and four framework regions (FRs including FR1, FR2, FR3, FR4), such as a VHH, a humanized VHH or a camelized VH (such as a camelized human VH) or generally a sequence optimized VHH (such as e.g., optimized for chemical stability and/or solubility, maximum overlap with known human framework regions and maximum expression), which is capable of binding to a specific antigen. The terms “nanobody” and “single-domain antibody” are used herein in its broadest form to include variants that may have various amino acid substitutions (e.g., conservative substitutions) and also functional “fragment” or “antigen binding fragment” of the nanobody as long as the fragment retains binding to the specific antigen.

A sdAb, or antigen binding fragment thereof, has a native or inherent binding affinity for its target antigen. The sdAb, or antigen binding fragment thereof, may be incorporated into a recombinant protein described herein as a targeting domain, and accordingly, provide the recombinant protein a native or inherent binding affinity for the target antigen (e.g., when unmasked). The recombinant protein may further comprise additional domain(s) as described herein.

Certain embodiments of the invention provide a recombinant protein comprising 1) a sdAb, or an antigen binding fragment thereof, and 2) a masking polypeptide that is operably linked to the sdAb, or an antigen binding fragment thereof. In certain embodiments, the sdAb, or an antigen binding fragment thereof, is operably linked to the masking polypeptide via a protease-sensitive polypeptide linker. Due to the presence of the masking polypeptide, the binding affinity of the sdAb for its target antigen is reduced. In certain embodiments, prior to unmasking, the binding between the masked sdAb and its target antigen is reduced by at least 15%, 20%, 30%, 40%, 50%, 60%, 70%, 80%, 90% or more (e.g., reduced by at least 1 fold, 5 fold, 10 fold, 15 fold, 20 fold, 25 fold, 30 fold, 35 fold, 40 fold, or more) as compared to a corresponding unmasked sdAb control.

As used herein, the term “masking polypeptide” refers to a polypeptide segment that (by its presence in the recombinant protein) reduces the binding affinity of the recombinant protein for the target antigen. However, the masking polypeptide may be removed from the recombinant protein and the binding affinity of the recombinant protein for the target antigen is restored. Namely, the recombinant protein does not exhibit the native or inherent binding affinity or capability for its target antigen in the presence of the masking polypeptide comprised within the recombinant protein. The recombinant protein could be activated by shedding the masking polypeptide to regain the binding affinity for its target antigen. In certain embodiments, the binding between the recombinant protein and its target antigen is reduced by at least 15%, 20%, 30%, 40%, 50%, 60%, 70%, 80%, 90% or more (e.g., reduced by at least 1 fold, 5 fold, 10 fold, 15 fold, 20 fold, 25 fold, 30 fold, 35 fold, 40 fold, or more) as compared to a corresponding unmasked recombinant protein control.

In certain embodiments, the recombinant protein comprises a masked sdAb. In certain embodiments, the recombinant protein consists of a masked sdAb.

In certain embodiments, the masking polypeptide is operably linked to the N-terminus of the sdAb, or antigen binding fragment thereof.

In certain embodiments, the masking polypeptide is a steric masking polypeptide and/or an affinity masking polypeptide.

In certain embodiments, the masking polypeptide is a steric masking polypeptide.

In certain embodiments, the masking polypeptide reduces the binding affinity of the recombinant protein for the target antigen through steric hindrance.

As used herein, the terms “steric hindrance” refers to a phenomenon wherein the bulk and physical presence of a group or domain in a recombinant protein reduces or denies the targeting domain present in the recombinant protein (i.e., present in the sdAb) access to its target antigen.

In certain embodiments, the masking polypeptide comprises a human histidine nucleotide triad binding protein 1 (hHINT1) polypeptide (see, e.g., NCBI accession number P49773.2 and 5IPE_A). Thus, in certain embodiments, the masking polypeptide comprises an amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to a hHINT1 polypeptide.

In certain embodiments, the masking polypeptide comprises an amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 3 or 31. In certain embodiments, the masking polypeptide comprises an amino acid sequence having at least 75% sequence identity to SEQ ID NO: 3 or 31. In certain embodiments, the masking polypeptide comprises an amino acid sequence having at least 80% sequence identity to SEQ ID NO: 3 or 31. In certain embodiments, the masking polypeptide comprises an amino acid sequence having at least 85% sequence identity to SEQ ID NO: 3 or 31. In certain embodiments, the masking polypeptide comprises an amino acid sequence having at least 90% sequence identity to SEQ ID NO: 3 or 31. In certain embodiments, the masking polypeptide comprises an amino acid sequence having at least 95% sequence identity to SEQ ID NO: 3 or 31. In certain embodiments, the masking polypeptide comprises the amino acid sequence of SEQ ID NO: 3 or 31. In certain embodiments, the masking polypeptide consists of the amino acid sequence of SEQ ID NO: 3 or 31.

In certain embodiments, the masking polypeptide is an affinity masking polypeptide. Normally, in its naked or unmasked format, an unhindered sdAb, or antigen binding fragment thereof, can bind with its target antigen as intermolecular binding partners. The sdAb, or antigen binding fragment thereof, binds a specific location of the target antigen. Such specific residues or segment of the target antigen bound by the sdAb, or antigen binding fragment thereof, is herein referred to as an “epitope”. Thus, in certain embodiments, the masking polypeptide comprises an epitope polypeptide sequence that is capable of specifically binding to the sdAb, or antigen binding fragment thereof. Thus, within a recombinant protein, the masking polypeptide may comprise an epitope polypeptide sequence that may function as an intramolecular binding partner for the targeting domain (i.e., the sdAb, or antigen binding fragment thereof). Accordingly, when comprised within the recombinant protein, the masking polypeptide comprising an epitope polypeptide sequence is capable of competitively inhibiting the intermolecular binding between the recombinant protein with a separate target antigen. In certain embodiments, the masking polypeptide comprises a tumor associated antigen (TAA) derived epitope polypeptide sequence that is capable of specifically binding to an anti-TAA sdAb. In certain embodiments, the masking polypeptide comprises a CD3 derived epitope polypeptide sequence that is capable of specifically binding to an anti-CD3 sdAb.

In certain embodiments, the masking polypeptide comprises a human CD3ε (CD3 epsilon chain) (see NCBI accession number P07766.2) derived polypeptide sequence, such as a CD3 epsilon chain fragment. In certain embodiments, the CD3 epsilon chain fragment comprises an epitope that is recognized by an anti-CD3 sdAb. Thus, in certain embodiments, the masking polypeptide comprises an amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to a human CD3 epsilon chain fragment sequence.

In certain embodiments, the masking polypeptide comprises an amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 10. In certain embodiments, the masking polypeptide comprises an amino acid sequence having at least 75% sequence identity to SEQ ID NO: 10. In certain embodiments, the masking polypeptide comprises an amino acid sequence having at least 80% sequence identity to SEQ ID NO: 10. In certain embodiments, the masking polypeptide comprises an amino acid sequence having at least 85% sequence identity to SEQ ID NO: 10. In certain embodiments, the masking polypeptide comprises an amino acid sequence having at least 90% sequence identity to SEQ ID NO: 10. In certain embodiments, the masking polypeptide comprises an amino acid sequence having at least 95% sequence identity to SEQ ID NO: 10. In certain embodiments, the masking polypeptide comprises the amino acid sequence of SEQ ID NO: 10. In certain embodiments, the masking polypeptide consists of the amino acid sequence of SEQ ID NO: 10.

In certain embodiments, the masking polypeptide has a length of about 5 to 180, or 10 to 160 amino acids (aa) in length. In certain embodiments, the masking polypeptide has a length of about 15 to 150 amino acids (aa) in length. In certain embodiments, the masking polypeptide has a length of about 20 to 140 amino acids (aa) in length. In certain embodiments, the masking polypeptide has a length of about 25 to 135 amino acids (aa) in length. In certain embodiments, the masking polypeptide has a length of about 30 to 130 amino acids (aa) in length. In certain embodiments, the masking polypeptide has a length of about 10 to 130, 20 to 130, 30 to 130, 40 to 130, 50 to 130, 60 to 130, 70 to 130, 80 to 130, 90 to 130, 100 to 130, 110 to 130, 120 to 130, 125 to 130 amino acids (aa) in length. In certain embodiments, the masking polypeptide has a length of about 26 to 28 amino acids (aa) in length. In certain embodiments, the masking polypeptide has a length of about 126 to 129 amino acids (aa) in length. In certain embodiments, the masking polypeptide has a length of about 10 to 30, 11 to 29, 12 to 28, 13 to 27, 14 to 26, or 15 to 25 amino acids (aa) in length.

The recombinant protein described herein may comprise one or more linkers between different domains (e.g., linking the masking polypeptide to the sdAb, or an antigen binding fragment thereof; or linking the sdAb, or an antigen binding fragment thereof, to an additional domain/element present in the recombinant protein). In certain embodiments, the linker is a flexible peptide or polypeptide linker. In certain embodiments, the linker is a glycine linker. In certain embodiments, the linker is a glycine rich linker (e.g., more than 60% of the linker sequence is glycine). In certain embodiments, the linker is a glycine-serine linker. In certain embodiments, the linker comprises G, GG, GSG, GGS, GGGS (SEQ ID NO: 41), GGGGS (SEQ ID NO: 15), GGSGGS (SEQ ID NO: 42), GSSGSS (SEQ ID NO: 43), or combination or repetition thereof. In certain embodiments, the linker is a glycine or glycine-serine linker as described in Table 1. In certain embodiments, each linker independently has a length of about 1 to 30, 2 to 29, 3 to 28, 4 to 27, 5 to 26, or 6 to 25 amino acids (aa) in length. In certain embodiments, a linker has a length of about 3 to 20, 10 to 19, 13 to 18, or 15 to 17 amino acids (aa) in length.

In certain embodiments, the linker is a cleavable linker. For example, in certain embodiments, the linker is a protease-sensitive polypeptide linker. The term “protease-sensitive polypeptide linker” refers to a linker sequence comprising an enzymatically cleavable polypeptide segment that is a substrate polypeptide sequence recognized by a proteolytic enzyme. The protease-sensitive polypeptide linker sequence may further comprise additional segment(s), for example, a flexible linker sequence such as a glycine, glycine rich, or glycine-serine sequence.

In certain embodiments, the protease-sensitive polypeptide linker is capable of being cleaved by a proteolytic enzyme that is expressed or overexpressed within a solid tumor. In certain embodiments, the protease-sensitive polypeptide linker is capable of being cleaved by a matrix metalloproteinase (MMP). In certain embodiments, the proteolytic enzyme (e.g., a MMP) is expressed or overexpressed by a cancer cell or other cells located within tumor microenvironment. In certain embodiments, the MMP is MMP2, MMP7, MMP9, or MMP13. In certain embodiments, the MMP is MMP2.

In certain embodiments, the protease-sensitive polypeptide linker comprises a MMP substrate sequence. MMP substrate sequences are known in the art and described herein, for example, the entire content of B. Ratnikov, et al., Proc Natl Acad Sci USA. 2014 Oct. 7; 111(40):E4148-55 is incorporated by reference herein. In certain embodiments, the protease-sensitive polypeptide linker comprises a MMP2 substrate sequence. In certain embodiments, the protease-sensitive polypeptide linker comprises GPLGVR (SEQ ID NO:4).

In certain embodiments, the protease-sensitive polypeptide linker comprises an amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 5. In certain embodiments, the protease-sensitive polypeptide linker comprises an amino acid sequence having at least 75% sequence identity to SEQ ID NO: 5. In certain embodiments, the protease-sensitive polypeptide linker comprises an amino acid sequence having at least 80% sequence identity to SEQ ID NO: 5. In certain embodiments, the protease-sensitive polypeptide linker comprises an amino acid sequence having at least 85% sequence identity to SEQ ID NO: 5. In certain embodiments, the protease-sensitive polypeptide linker comprises an amino acid sequence having at least 90% sequence identity to SEQ ID NO: 5. In certain embodiments, the protease-sensitive polypeptide linker comprises an amino acid sequence having at least 95% sequence identity to SEQ ID NO: 5. In certain embodiments, the protease-sensitive polypeptide linker comprises the amino acid sequence of SEQ ID NO: 5. In certain embodiments, the protease-sensitive polypeptide linker consists of the amino acid sequence of SEQ ID NO: 5.

In certain embodiments, the protease-sensitive polypeptide linker comprises an amino acid sequence comprising GPLGVR (SEQ ID NO:4) and having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 5.

In certain embodiments, the sdAb, or antigen binding fragment thereof, has affinity for a target antigen protein (e.g., a cell membrane anchored protein). In certain embodiments, the targeting domain (i.e., the sdAb, or antigen binding fragment thereof), may have affinity for a cell surface protein, such as a cell membrane anchored protein. In certain embodiments, the target antigen protein is a tumor associated antigen (TAA) expressed or overexpressed by a cancer cell (e.g., a malignant cell, or a cancer stem cell (CSC)). In certain embodiments, the target antigen protein is human prostate-specific membrane antigen (PSMA), HER2, EGFR, EpCAM, CD133, or αvβ3.

In certain embodiments, the sdAb is an anti-PSMA sdAb, or an antigen binding fragment thereof. In certain embodiments, the sdAb is an anti-EGFR sdAb, or an antigen binding fragment thereof. In certain embodiments, the sdAb is an anti-EpCAM sdAb, or an antigen binding fragment thereof. In certain embodiments, the sdAb is an anti-CD133 sdAb, or an antigen binding fragment thereof. In certain embodiments, the sdAb is an anti-αvβ3 sdAb, or an antigen binding fragment thereof.

In certain embodiments, the anti-PSMA sdAb, an antigen binding fragment thereof, comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO:2. In certain embodiments, the anti-PSMA sdAb, an antigen binding fragment thereof, comprises an amino acid sequence having at least 85% sequence identity to SEQ ID NO:2. In certain embodiments, the anti-PSMA sdAb, an antigen binding fragment thereof, comprises an amino acid sequence having at least 90% sequence identity to SEQ ID NO:2. In certain embodiments, the anti-PSMA sdAb, an antigen binding fragment thereof, comprises an amino acid sequence having at least 95% sequence identity to SEQ ID NO:2. In certain embodiments, the anti-PSMA sdAb comprises the amino acid sequence of SEQ ID NO:2. In certain embodiments, the anti-PSMA sdAb consists of the amino acid sequence of SEQ ID NO:2.

In certain embodiments, the recombinant protein (hHINT1 masked anti-PSMA sdAb) comprises an amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO:8 or 9. In certain embodiments, the recombinant protein comprises an amino acid sequence having at least 75% sequence identity to SEQ ID NO: 8 or 9. In certain embodiments, the recombinant protein comprises an amino acid sequence having at least 80% sequence identity to SEQ ID NO: 8 or 9. In certain embodiments, the recombinant protein comprises an amino acid sequence having at least 85% sequence identity to SEQ ID NO: 8 or 9. In certain embodiments, the recombinant protein comprises an amino acid sequence having at least 90% sequence identity to SEQ ID NO: 8 or 9. In certain embodiments, the recombinant protein comprises an amino acid sequence having at least 95% sequence identity to SEQ ID NO: 8 or 9. In certain embodiments, the recombinant protein comprises the amino acid sequence of SEQ ID NO: 8 or 9. In certain embodiments, the recombinant protein consists of the amino acid sequence of SEQ ID NO: 8 or 9.

In certain embodiments, the target antigen of the sdAb, or antigen binding fragment thereof, is a cell surface protein expressed by an immune cell. In certain embodiments, the target antigen of the sdAb, or antigen binding fragment thereof, is a cell surface protein expressed by a T cell. For example, in certain embodiments, the sdAb is an anti-CD3 sdAb, or an antigen binding fragment thereof.

In certain embodiments, the anti-CD3 sdAb, an antigen binding fragment thereof, comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO:1. In certain embodiments, the anti-CD3 sdAb, an antigen binding fragment thereof, comprises an amino acid sequence having at least 85% sequence identity to SEQ ID NO:1. In certain embodiments, the anti-CD3 sdAb, an antigen binding fragment thereof, comprises an amino acid sequence having at least 90% sequence identity to SEQ ID NO:1. In certain embodiments, the anti-CD3 sdAb, an antigen binding fragment thereof, comprises an amino acid sequence having at least 95% sequence identity to SEQ ID NO:1. In certain embodiments, the anti-CD3 sdAb comprises the amino acid sequence of SEQ ID NO:1. In certain embodiments, the anti-CD3 sdAb consists of the amino acid sequence of SEQ ID NO:1. In certain embodiments, the anti-CD3 sdAb, or an antigen binding fragment thereof, comprises one or more CDR amino acid sequences of the anti-CD3 sdAb (see the underlined CDR1, CDR2, and CDR3 sequences of SEQ ID NO:1). In particular, in certain embodiments, the anti-CD3 sdAb, or an antigen binding fragment thereof, comprises one or more CDR amino acid sequences selected from the group consisting of: a CDR1 sequence comprising DYGMS (SEQ ID NO:38); a CDR2 sequence comprising DISWNGGSTYYADSVKG (SEQ ID NO:39); and a CDR3 sequence comprising MGEGGWGANDY (SEQ ID NO:40).

In certain embodiments, the recombinant protein (hHINT1 masked anti-CD3 sdAb) comprises an amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO:6 or 7. In certain embodiments, the recombinant protein comprises an amino acid sequence having at least 75% sequence identity to SEQ ID NO: 6 or 7. In certain embodiments, the recombinant protein comprises an amino acid sequence having at least 80% sequence identity to SEQ ID NO: 6 or 7. In certain embodiments, the recombinant protein comprises an amino acid sequence having at least 85% sequence identity to SEQ ID NO: 6 or 7. In certain embodiments, the recombinant protein comprises an amino acid sequence having at least 90% sequence identity to SEQ ID NO: 6 or 7. In certain embodiments, the recombinant protein comprises an amino acid sequence having at least 95% sequence identity to SEQ ID NO: 6 or 7. In certain embodiments, the recombinant protein comprises the amino acid sequence of SEQ ID NO: 6 or 7. In certain embodiments, the recombinant protein consists of the amino acid sequence of SEQ ID NO: 6 or 7.

In certain embodiments, the recombinant protein (epitope peptide masked anti-CD3 sdAb) comprises an amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO:11 or 12. In certain embodiments, the recombinant protein comprises an amino acid sequence having at least 75% sequence identity to SEQ ID NO: 11 or 12. In certain embodiments, the recombinant protein comprises an amino acid sequence having at least 80% sequence identity to SEQ ID NO: 11 or 12. In certain embodiments, the recombinant protein comprises an amino acid sequence having at least 85% sequence identity to SEQ ID NO: 11 or 12. In certain embodiments, the recombinant protein comprises an amino acid sequence having at least 90% sequence identity to SEQ ID NO: 11 or 12. In certain embodiments, the recombinant protein comprises an amino acid sequence having at least 95% sequence identity to SEQ ID NO: 11 or 12. In certain embodiments, the recombinant protein comprises the amino acid sequence of SEQ ID NO: 11 or 12. In certain embodiments, the recombinant protein consists of the amino acid sequence of SEQ ID NO: 11 or 12.

In certain embodiments, the recombinant protein comprises one or more optional peptide or polypeptide tag(s) at the N-terminus and/or C-terminus of the protein (e.g., a His6 tag (SEQ ID NO: 20) or FLAG tag, or c-Myc tag for purification or detection of the protein).

In certain embodiments, the recombinant protein is a masked sdAb. In certain embodiments, the recombinant protein further comprises a human immunoglobulin constant region sequence, including but not limited to a Fc domain (e.g., Fc domain from IgG1, IgG2, IgG3, IgG4, IgA, IgE, IgD, or IgM).

In certain embodiments, a therapeutic agent or detectable agent is operably linked to the recombinant protein. In certain embodiments, the therapeutic agent or detectable agent is linked to the recombinant protein via a linker group (e.g., via polypeptide linker or a chemical linking group). In certain embodiments, the therapeutic agent or detectable agent is directly linked to the recombinant protein (e.g., via a peptide bond).

In certain embodiments, the therapeutic or detectable agent is operably linked to the recombinant protein via a peptide bond or a polypeptide linker. In certain embodiments, the therapeutic or detectable agent is a polypeptide or protein (e.g., an enzyme or a protein toxin).

In certain embodiments, the therapeutic or detectable agent is directly linked to the recombinant protein (e.g., on a lysine or cysteine residue of the recombinant protein). In certain embodiments, the therapeutic or detectable agent is operably linked to the recombinant protein via a chemical linking group. Thus, certain embodiments provide a conjugate comprising a recombinant polypeptide described herein linked to a therapeutic or detectable agent. SdAb conjugates for cancer therapy or imaging detection are known in the art and described herein, for example, the entire content of W. Kang et al., Technol Cancer Res Treat. 2021; 20: 15330338211010117 is incorporated by reference herein.

In certain embodiments, the therapeutic or detectable agent is a small molecule with a molecular weight of no more than 1000 g/mol. In certain embodiments, the therapeutic or detectable agent is a therapeutic agent. In certain embodiments, the therapeutic agent is a cytotoxic drug. In certain embodiments, the therapeutic or detectable agent is a detectable agent. In certain embodiments, the therapeutic or detectable agent is a fluorescent dye. In certain embodiments, the therapeutic or detectable agent is a radionuclide.

In certain embodiments, the recombinant protein is conjugated to a cytotoxic drug.

In certain embodiments, the recombinant protein is not conjugated to a cytotoxic drug.

In certain embodiments, the recombinant protein further comprises one or more intracellular signaling domain(s). In certain embodiments, the recombinant protein is a chimeric antigen receptor expressed by an immune cell such as a T cell or NK cell (as a transmembrane protein). SdAbs incorporated as part of a chimeric antigen receptor expressed by a T cell (CAR-T cell) for cancer therapy are known in the art and described herein, for example, the entire content of Fengzhen Mo et al., Signal Transduct Target Ther. 2021 Feb. 25; 6(1):80; and C. Bao et al., Biomolecules 2021, 11(2), 238 are incorporated by reference herein.

In certain embodiments, the recombinant protein described herein further comprises a domain that may facilitate dimerization (e.g., an IgG Fc domain) of two polypeptide chains.

A recombinant protein comprising a masked sdAb as described herein may also be comprised within a multivalent protein. For example, certain embodiments of the invention also provide multivalent sdAbs (e.g., bivalent, trivalent, tetravalent, pentavalent or higher valence multivalent sdAbs), wherein at least one of the sdAbs is masked. Thus, certain embodiments of the invention provide a protein comprising two or more independently selected sdAbs as described herein, wherein the sdAbs are operably linked to each other (e.g., to form a dimer, trimer, tetramer, pentamer or higher valence multimer sdAb) and wherein at least one of the sdAbs is masked. In certain embodiments, a multivalent sdAb or binding protein as described herein is a homo-multimer (e.g., dimer, trimer, tetramer or pentamer). In certain embodiments, a multivalent sdAb or binder protein as described herein is a hetero-multimer (e.g., dimer, trimer, tetramer or pentamer). In certain embodiments, a multivalent sdAb or binder protein as described herein is a bispecific binder protein comprising two different sdAbs. In certain embodiments, one of the sdAbs present in a multivalent protein is masked. In certain embodiments, more than one of the sdAbs present in a multivalent protein are masked. In certain embodiments, all of the sdAbs present in a multivalent protein are masked.

In certain embodiments, the two or more sdAbs are operably linked via a linker group (e.g., a peptide linker group), disulfide bond(s) and/or by non-covalent interactions. In certain embodiments, the two or more sdAbs are operably linked via oligomerization of tag polypeptides (e.g., multimerization tags, such as a dimerization tag, trimerization tag, tetramerization tag, etc.).

In certain embodiments, the two or more sdAbs are operably linked via a linker group. The nature of the linker group is not critical, provided that the linker group does not interfere with the function of the sdAb. In certain embodiments, the linker group is a peptide linker group. In certain embodiments, the peptide linker is a glycine-serine rich linker.

In certain embodiments, two independently selected sdAbs are linked via a linker group (e.g., a peptide linker group) to form a dimeric sdAb. In certain embodiments, three independently selected sdAbs are linked via two linker groups (e.g., two peptide linker groups) to form a trimeric sdAb. In certain embodiments, four independently selected sdAbs are linked via three linker groups (e.g., three peptide linker groups) to form a tetrameric sdAb. In certain embodiments, five independently selected sdAbs are linked via four linker groups (e.g., four peptide linker groups) to form a pentameric sdAb.

In certain embodiments, the two or more sdAbs are operably linked via oligomerization of tag polypeptides. For example, a sdAb as described herein may be operably linked to a tag polypeptide to form a sdAb-tag fusion polypeptide, wherein the tag polypeptide is capable of oligomerizing. Accordingly, two or more sdAb-tag fusion polypeptides may be operably linked to form a dimer, trimer, tetramer, pentamer or a higher valence multimer via polypeptide tag-mediated oligomerization.

In certain embodiments, the sdAb and tag polypeptide are linked through a peptide linker to form the sdAb-tag fusion polypeptide. In certain embodiments, a sdAb and tag polypeptide are directly linked without an intervening peptide linker to form the sdAb-tag fusion polypeptide.

In certain embodiments, the tag polypeptide is a human Fc sequence. Accordingly, certain embodiments of the invention provide a protein comprising: two independently selected sdAb-Fc fusion polypeptides as described herein, wherein the two Fc polypeptides are linked to form a dimer (e.g., linked by a covalent bond, such as a disulfide bond, or by non-covalent interactions such as electrostatic interactions, hydrogen bonding, etc.), and wherein at least one of the sdAb-Fc fusion polypeptides are masked.

In certain embodiments, the two sdAb-Fc fusion polypeptides are the same. In certain embodiments, the two sdAb-Fc fusion polypeptides are different. In certain embodiments, sdAb-Fc polypeptides as described herein can form homo-dimers. In certain embodiments, sdAb-Fc polypeptides as described herein can form hetero-dimers. In certain embodiments, sdAb-Fc polypeptides as described herein can form bispecific hetero-dimers.

In certain other embodiments, a single sdAb of the invention is operably linked to an Fc dimer.

In certain embodiments, the recombinant protein described herein further comprises a domain that facilitates oligomerization or self-assembling into an assembly (e.g., a nanoring assembly). In certain embodiments, the recombinant protein is a monomer unit capable of oligomerizing. In certain embodiments, the recombinant protein is a monomer unit that is capable of assembling into or participating in the formation of a supramolecular structure or assembly. In certain embodiments, the recombinant protein is a monomer unit of a nanoring comprising a plurality of same or different monomer units.

In certain embodiments, the recombinant protein described herein further comprises one or more DHFR domains (e.g., a DHFR domain as described below). For example, as described below, such a recombinant protein may be capable of assembling into a chemically self-assembled antibody nanoring (CSAN) in the presence of bisMTX compound.

Recombinant Protein as a Monomer Unit Capable of Oligomerizing into a Chemically Self-Assembled Nanoring (CSAN)

As described herein, a recombinant protein of the invention may be incorporated into an assembly, such as a chemically self-assembled nanoring (CSAN), e.g., that might be used for diagnostic or therapeutic purposes.

Thus, certain embodiments of the invention provide a chemically self-assembled nanoring (CSAN) as described herein. As used herein, the term “CSAN” refers to an oligomeric nanoring comprising a plurality of (e.g., eight) monomeric polypeptide units having affinity for a chemical dimerizer so that the nanoring is assembled through the binding between monomer polypeptide units and the chemical dimerizer, wherein each monomer polypeptide unit binds two chemical dimerizers and each chemical dimerizer binds two monomer polypeptide units to close the loop of the nanoring. As described below, certain recombinant proteins as described herein may function as a monomeric polypeptide unit of the CSAN, and may be referred herein as a recombinant protein monomer unit. A “monomeric polypeptide unit”, “monomer polypeptide unit” and “recombinant protein monomer unit” are used interchangeably and as used herein broadly refer to a polypeptide (through its affinity for a chemical dimerizer) that is capable of forming a nanoring or being incorporated into a nanoring. As described below, a CSAN may comprise the same or different types of monomeric polypeptide units (i.e., homo-monomer based CSAN, or hetero-monomer based CSAN). In certain embodiments, a monomer polypeptide unit may comprise a masked sdAb (i.e., a masked sdAb as described herein, or an antigen binding fragment thereof). In certain embodiments, a monomer polypeptide unit may comprise a sdAb (i.e., a sdAb, or an antigen binding fragment thereof) and does not comprise a masking polypeptide.

In certain embodiments, the CSAN comprises eight recombinant protein monomer units (e.g., same or different monomer units). In certain embodiments, the CSAN comprises at least one recombinant protein monomer unit as described herein. In certain embodiments, the CSAN comprises a plurality (e.g., 2, 3, 4, 5, 6, 7, or 8) of recombinant protein monomer units as described herein.

In certain embodiments, the chemical stabilizer may be a bisMTX compound. Certain bisMTX compounds are known in the art. In certain embodiments, the bisMTX compound is a bisMTX compound described in, e.g., Carlson, J. C. T., et al. J. Am. Chem. Soc. 2006, 128, 7630-7638; Fegan, A., et al. Mol. Pharmaceutics. 2012, 9, 3218-3227; Li, Q., et al., J. Am. Chem. Soc. 2010, 132, 17247-17257; Shah, R, et al., Mol. Pharmaceutics. 2016, 13 (7), 2193-2203; Gangar, A., et al., J. Am. Chem. Soc. 2012, 134, 2895-2897; Shen, J., et al., J. Am. Chem. Soc. 2015, 137, 10108-10111; Qing, L., et al., Angew. Chem. Int. Ed. 2008, 47, 10179-10182; Gangar, A., et al., Mol. Pharmaceutics. 2013, 10, 3514-3518; Gabrielse, K., et al., Angew. Chem. Int. Ed. 2014, 53, 5112-5116; US Patent publication US2015-0343082, US Patent publication US2015-0017189, U.S. Pat. No. 8,236,925 or U.S. Pat. No. 8,580,921 (these documents are incorporated by reference herein for all purposes). The plurality of bisMTX compounds may consist of a single type of bisMTX or may be a mixture of different types of compounds (e.g., 2, 3, 4, 5 or more types of compounds).

In certain embodiments, the monomeric polypeptide units of the CSAN comprise one or more dihydrofolate reductase (DHFR) polypeptides, which have affinity for bisMTX compounds. For example, in certain embodiments, the nanoring comprises multiple fusion proteins, each comprising two subunits of dihydrofolate reductase (DHFR) joined by a peptide linker of variable length (e.g., 1-13 amino acids) and may be further fused to other domains (e.g., to a polypeptide described herein) and peptides (see, e.g., Carlson, J. C. T., et al. J. Am. Chem. Soc. 2006, 128, 7630-7638; Fegan, A., et al. Mol. Pharmaceutics. 2012, 9, 3218-3227; Li, Q., et al., J. Am. Chem. Soc. 2010, 132, 17247-17257; Shah, R, et al., Mol. Pharmaceutics. 2016, 13 (7), 2193-2203; Gangar, A., et al., J. Am. Chem. Soc. 2012, 134, 2895-2897; Shen, J., et al., J. Am. Chem. Soc. 2015, 137, 10108-10111; Qing, L., et al., Angew. Chem. Int. Ed. 2008, 47, 10179-10182; Gangar, A., et al., Mol. Pharmaceutics. 2013, 10, 3514-3518; Gabrielse, K., et al., Angew. Chem. Int. Ed. 2014, 53, 5112-5116. These documents are incorporated by reference in their entirety for all purposes).

Thus, certain embodiments provide a CSAN comprising at least one recombinant protein monomer unit that comprises a masked sdAb as described herein (i.e., a masked sdAb as described herein, or antigen binding fragment thereof). For example, a CSAN may be formed when certain recombinant proteins of the invention (e.g., one or more) are contacted with a chemical dimerizer (e.g., bis-methotrexate). As described herein, the monomeric polypeptide units having affinity for a chemical dimerizer may be a recombinant protein as described herein, which comprises one or more DHFR domains (e.g., a DHFR domain). Thus, in certain embodiments, the CSAN comprises a plurality of recombinant protein monomer units as described herein and a plurality of bisMTX compounds, wherein at least one recombinant protein monomer unit comprises a masked sdAb as described herein.

As noted above, the term “DHFR” refers to a dihydrofolate reductase polypeptide. Such a polypeptide may be included as a domain or subunit of certain embodiments of the recombinant protein described herein. DHFR domains are capable of binding a BisMTX compound. Accordingly, inclusion of DHFR domains in a recombinant protein described herein may facilitate the oligomerization of recombinant protein into a CSAN as described herein. In certain embodiments, the DHFR domain comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identity to a DHFR sequence (e.g., see NCBI accession number 5UII_A).

Thus, in certain embodiments, a recombinant protein described herein comprises one or more DHFR domains. In certain embodiments, the DHFR domain comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO:13 or 36. In certain embodiments, the DHFR domain comprises an amino acid sequence having at least 85% sequence identity to SEQ ID NO:13 or 36. In certain embodiments, the DHFR domain comprises an amino acid sequence having at least 90% sequence identity to SEQ ID NO:13 or 36. In certain embodiments, the DHFR domain comprises an amino acid sequence having at least 95% sequence identity to SEQ ID NO:13 or 36. In certain embodiments, the DHFR domain comprises the amino acid sequence of SEQ ID NO:13 or 36. In certain embodiments, the DHFR domain consists of the amino acid sequence of SEQ ID NO:13 or 36.

In certain embodiments, the recombinant protein described herein comprises a first DHFR domain and a second DHFR domain (also referred to as DHFR² domain). An example of a DHFR² domain is SEQ ID NO:14 or 37, which is also referenced herein as 1DD. Thus, in certain embodiments, the DHFR² domain comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO:14 or 37. In certain embodiments, the DHFR² domain comprises an amino acid sequence having at least 85% sequence identity to SEQ ID NO:14 or 37. In certain embodiments, the DHFR² domain comprises an amino acid sequence having at least 90% sequence identity to SEQ ID NO:14 or 37. In certain embodiments, the DHFR² domain comprises an amino acid sequence having at least 95% sequence identity to SEQ ID NO:14 or 37. In certain embodiments, the DHFR² domain comprises the amino acid sequence of SEQ ID NO:14 or 37. In certain embodiments, the DHFR² domain consists of the amino acid sequence of SEQ ID NO:14 or 37.

In certain embodiments, the recombinant protein monomer unit is a masked sdAb-DHFR² polypeptide, wherein the masked sdAb is operably linked to the DHFR² domain (e.g., comprising from N terminal to C terminal: a masking polypeptide, a protease-sensitive polypeptide linker, a sdAb, or an antigen binding fragment thereof, and a DHFR² domain). In certain embodiments, the recombinant protein monomer unit is a masked anti-CD3 sdAb-DHFR² polypeptide. In certain embodiments, the recombinant protein monomer unit is a masked anti-TAA sdAb-DHFR² polypeptide. For example, in certain embodiments, the recombinant protein monomer unit is a masked anti-PSMA sdAb-DHFR² polypeptide.

In certain embodiments, the CSAN comprises a plurality of recombinant protein monomer units of the same type (i.e., comprises homo-monomers).

In certain embodiments, the CSAN is a monospecific CSAN that has affinity for one target antigen (e.g., a T cell antigen such as CD3, or a TAA such as PSMA).

In certain embodiments, the CSAN comprises a plurality of recombinant protein monomer unit of different types (i.e., comprises hetero-monomers).

In certain embodiments, the CSAN is a bispecific CSAN that has affinity for two target antigens (e.g., for 1) a T cell antigen, such as CD3; and 2) a tumor associated antigen (TAA), such as PSMA, HER2, or EGFR, etc.).

As noted above, certain embodiments provide a CSAN comprising at least one recombinant protein monomer unit that comprises a masked sdAb as described herein. For example, in certain embodiments, the CSAN comprises 1, 2, 3, 4, 5, 6, 7, or 8 recombinant protein monomer unit comprising a masked sdAb (e.g., anti-CD3 or anti-TAA) as described herein.

In certain embodiments, the CSAN comprises at least two different types of recombinant protein monomer units. In certain embodiments, the CSAN (e.g., a bispecific CSAN) comprises a) one or more (e.g., 1, 2, 3, 4, 5, 6, or 7) first recombinant protein monomer units, and b) one or more (e.g., 1, 2, 3, 4, 5, 6, or 7) second recombinant protein monomer units. In certain embodiments, the first recombinant protein monomer unit comprises a masked sdAb as described herein. In certain embodiments, the CSAN is a bispecific CSAN, wherein the second recombinant protein monomer unit comprises a targeting domain, such as a sdAb, a single-chain variable fragment (scFv), an affibody, a human tenth type III fibronectin scaffold (Fn3), or a targeting peptide such as Arginine-Glycine-Aspartate (RGD). The term “targeting domain” as used herein for this second recombinant protein monomer unit of a bispecific CSAN refers to a peptide or polypeptide as a domain or subunit of the protein, which has affinity for a target (e.g., a cell membrane anchored protein). In certain embodiments, the targeting domain may have affinity for a cell surface protein, such as a cell membrane anchored protein. In certain embodiments, the polypeptide may be an antibody mimetic such as affibody or human tenth type III fibronectin scaffold (Fn3). In certain embodiments, the polypeptide may be a sdAb, or an antigen binding fragment. In certain embodiments, the polypeptide may be a scFv. In certain embodiments, the peptide comprises Arg-Gly-Asp (RGD) sequence or motif. In certain embodiments, this second recombinant protein monomer unit comprises, from N-terminal to C-terminal, a targeting domain and DHFR² domain. In certain embodiments, this second recombinant protein monomer unit comprises, from N-terminal to C-terminal, a DHFR² domain and a targeting domain (e.g., a DHFR² domain linked to scFv, such as 1DD-anti-CD3 scFv or SEQ ID NO:32 in Table 1).

Thus, in certain embodiments, the second recombinant protein monomer unit, or the assembly CSAN (e.g., bispecific CSAN), comprises or is linked or conjugated to a targeting domain (e.g., a sdAb, a single-chain variable fragment (scFv), an affibody, or human tenth type III fibronectin scaffold (Fn3), or a targeting peptide such as Arginine-Glycine-Aspartate (RGD)), that specifically binds a target antigen). In certain embodiments, the targeting domain of the second recombinant protein monomer unit may be a masked or unmasked sdAb. Thus, in certain embodiments, the CSAN is a bispecific CSAN comprising a plurality of (e.g., four) a first recombinant protein monomer unit and a plurality of (e.g., four) a second recombinant protein monomer unit, wherein the first and/or the second recombinant protein monomer unit comprises a masking polypeptide. Hence, in certain embodiments, the bispecific CSAN is a dual-masked CSAN. In certain embodiments, the bispecific CSAN is a mono-masked CSAN.

In certain embodiments, the CSAN comprises two different types of recombinant protein monomer units (e.g., one unit is anti-T cell antigen, and the other unit is anti-Tumor associated antigen). In certain embodiments, the CSAN comprises 1) anti-CD3 recombinant protein monomer unit (e.g., comprising anti-CD3 sdAb or anti-CD3 scFv), and 2) anti-TAA recombinant protein monomer unit (e.g., comprising anti-TAA sdAb or anti-TAA scFv).

In certain embodiments, the bispecific CSAN is a dual-masked CSAN (the affinity for both antigens are masked), wherein the first recombinant protein monomer unit comprises a masked sdAb and the second recombinant protein monomer unit comprises another masked sdAb. For example, in certain embodiments, the first recombinant protein monomer unit comprises a masked anti-TAA sdAb (e.g., anti-PSMA) and the second recombinant protein monomer unit comprises a masked anti-CD3 sdAb.

In certain embodiments, the bispecific CSAN is a mono-masked CSAN (the affinity for one antigen is masked and the affinity for the other antigen is not masked), wherein the first recombinant protein monomer unit comprises a masked sdAb as described herein and the second recombinant protein monomer unit comprises a targeting domain (e.g., sdAb, scFv, affibody, Fn3 or RGD peptide). For example, in certain embodiments, the first recombinant protein monomer unit comprises a masked anti-TAA sdAb as described herein and the second recombinant protein monomer unit comprises an anti-CD3 targeting domain (e.g., sdAb, scFv, affibody, or Fn3). In certain embodiments, the first recombinant protein monomer unit comprises a masked anti-CD3 sdAb as described herein and the second recombinant protein monomer unit comprises an unmasked anti-TAA sdAb.

In certain embodiments, the CSAN is an anti-CD3/anti-TAA bispecific CSAN, wherein the anti-CD3 monomer unit is a masked recombinant protein monomer unit. In certain embodiments, the CSAN is an anti-CD3/anti-TAA bispecific CSAN, wherein the anti-TAA monomer unit is a masked recombinant protein monomer unit. In certain embodiments, the ratio between two types of recombinant protein monomer units is 1:7, 1:3, 3:5, or 1:1. In certain embodiments, the ratio between two types of recombinant protein monomer units is 5:3, 3:1, or 7:1.

In certain embodiments, the CSAN is a bispecific CSAN wherein one monomer unit is masked, and the other monomer unit is unmasked. For example, in certain embodiments, the CSAN is a masked anti-CD3/unmasked anti-TAA bispecific CSAN. In certain embodiments, the CSAN is a masked anti-CD3/unmasked anti-PSMA bispecific CSAN. In certain embodiments, the CSAN is an unmasked anti-CD3/masked anti-TAA bispecific CSAN. In certain embodiments, the CSAN is an unmasked anti-CD3/masked anti-PSMA bispecific CSAN. Certain non-limiting, exemplary masked or unmasked protein sequences are described herein (see, e.g., Table 1, Example 1 and Example 2).

In certain embodiments, the binding between the monospecific CSAN and its target antigen is reduced by at least 15%, 20%, 30%, 40%, 50%, 60%, 70%, 80%, 90% or more (e.g., reduced by at least 1 fold, 5 fold, 10 fold, 15 fold, 20 fold, 25 fold, 30 fold, 35 fold, 40 fold, or more) as compared to the corresponding unmasked CSAN control.

In certain embodiments, the binding between the bispecific CSAN and one of its target antigen is reduced by at least 15%, 20%, 30%, 40%, 50%, 60%, 70%, 80%, 90% or more (e.g., reduced by at least 1 fold, 5 fold, 10 fold, 15 fold, 20 fold, 25 fold, 30 fold, 35 fold, 40 fold, or more) as compared to the corresponding unmasked bispecific CSAN control.

In certain embodiments, prior to protease cleavage or in an early period of contact (e.g., within 1, 2, 3, 4, 5, 6, 12, 24, 48, or 72 hours), the bispecific CSAN (e.g., anti-CD3/anti-TAA) mediated T cell killing of a target cell (e.g., a cancer cell) expressing a target antigen is reduced by at least 15%, 20%, 30%, 40%, 50%, 60%, 70%, 80%, 90% or more (e.g., reduced by at least 1 fold, 5 fold, 10 fold, 15 fold, 20 fold, 25 fold, 30 fold, 35 fold, 40 fold, or more) as compared to the corresponding unmasked CSAN control.

In certain embodiments, the bispecific CSAN (e.g., anti-CD3/anti-TAA) mediated T cell killing of a cell expressing a target antigen (e.g., healthy cell or a cell in healthy tissue) is reduced by at least 15%, 20%, 30%, 40%, 50%, 60%, 70%, 80%, 90% or more (e.g., reduced by at least 1 fold, 5 fold, 10 fold, 15 fold, 20 fold, 25 fold, 30 fold, 35 fold, 40 fold, or more) as compared to the corresponding unmasked CSAN control.

In certain embodiments, the bispecific CSAN (e.g., anti-CD3/anti-TAA) mediated T cell activation in a healthy tissue or outside tumor microenvironment is reduced by at least 15%, 20%, 30%, 40%, 50%, 60%, 70%, 80%, 90% or more (e.g., reduced by at least 1 fold, 5 fold, 10 fold, 15 fold, 20 fold, 25 fold, 30 fold, 35 fold, 40 fold, or more) as compared to the corresponding unmasked CSAN control.

In certain embodiments, the CSAN (e.g., bispecific CSAN) comprises a first recombinant protein comprising masked anti-TAA sdAb, and the CSAN further comprises a second recombinant protein comprising an anti-CD3 targeting domain (e.g., anti-CD3 scFv, affibody, Fn3, or sdAb).

In certain embodiments, the CSAN (e.g., bispecific CSAN) comprises a first recombinant protein comprising masked anti-CD3 sdAb, and the CSAN further comprises a second recombinant protein comprising an anti-TAA targeting domain (e.g., anti-TAA scFv, affibody, Fn3, or sdAb).

In certain embodiments, the recombinant protein monomer unit (hHINT1 masked anti-CD3-1DD) comprises an amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO:21. In certain embodiments, the recombinant protein comprises an amino acid sequence having at least 75% sequence identity to SEQ ID NO: 21. In certain embodiments, the recombinant protein comprises an amino acid sequence having at least 80% sequence identity to SEQ ID NO: 21. In certain embodiments, the recombinant protein comprises an amino acid sequence having at least 85% sequence identity to SEQ ID NO: 21. In certain embodiments, the recombinant protein comprises an amino acid sequence having at least 90% sequence identity to SEQ ID NO: 21. In certain embodiments, the recombinant protein comprises an amino acid sequence having at least 95% sequence identity to SEQ ID NO: 21. In certain embodiments, the recombinant protein comprises the amino acid sequence of SEQ ID NO: 21. In certain embodiments, the recombinant protein consists of the amino acid sequence of SEQ ID NO: 21.

In certain embodiments, the recombinant protein monomer unit (hHINT1 masked anti-PSMA-1DD) comprises an amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO:23. In certain embodiments, the recombinant protein comprises an amino acid sequence having at least 75% sequence identity to SEQ ID NO: 23. In certain embodiments, the recombinant protein comprises an amino acid sequence having at least 80% sequence identity to SEQ ID NO: 23. In certain embodiments, the recombinant protein comprises an amino acid sequence having at least 85% sequence identity to SEQ ID NO: 23. In certain embodiments, the recombinant protein comprises an amino acid sequence having at least 90% sequence identity to SEQ ID NO: 23. In certain embodiments, the recombinant protein comprises an amino acid sequence having at least 95% sequence identity to SEQ ID NO: 23. In certain embodiments, the recombinant protein comprises the amino acid sequence of SEQ ID NO: 23. In certain embodiments, the recombinant protein consists of the amino acid sequence of SEQ ID NO: 23.

In certain embodiments, the recombinant protein monomer unit (epitope peptide masked anti-CD3-1DD) comprises an amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO:25. In certain embodiments, the recombinant protein comprises an amino acid sequence having at least 75% sequence identity to SEQ ID NO: 25. In certain embodiments, the recombinant protein comprises an amino acid sequence having at least 80% sequence identity to SEQ ID NO: 25. In certain embodiments, the recombinant protein comprises an amino acid sequence having at least 85% sequence identity to SEQ ID NO: 25. In certain embodiments, the recombinant protein comprises an amino acid sequence having at least 90% sequence identity to SEQ ID NO: 25. In certain embodiments, the recombinant protein comprises an amino acid sequence having at least 95% sequence identity to SEQ ID NO: 25. In certain embodiments, the recombinant protein comprises the amino acid sequence of SEQ ID NO: 25. In certain embodiments, the recombinant protein consists of the amino acid sequence of SEQ ID NO: 25.

In certain embodiments, the recombinant protein monomer unit (1DD-anti-CD3 scFv) comprises an amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO:32. In certain embodiments, the recombinant protein comprises an amino acid sequence having at least 75% sequence identity to SEQ ID NO: 32. In certain embodiments, the recombinant protein comprises an amino acid sequence having at least 80% sequence identity to SEQ ID NO: 32. In certain embodiments, the recombinant protein comprises an amino acid sequence having at least 85% sequence identity to SEQ ID NO: 32. In certain embodiments, the recombinant protein comprises an amino acid sequence having at least 90% sequence identity to SEQ ID NO: 32. In certain embodiments, the recombinant protein comprises an amino acid sequence having at least 95% sequence identity to SEQ ID NO: 32. In certain embodiments, the recombinant protein comprises the amino acid sequence of SEQ ID NO: 32. In certain embodiments, the recombinant protein consists of the amino acid sequence of SEQ ID NO: 32.

In certain embodiments, the CSAN is an octameric nanoring comprising one or more types of recombinant protein monomer described herein.

In certain embodiments, the CSAN comprises one or more masked anti-CD3 sdAb-DHFR recombinant protein monomer unit(s) (e.g., SEQ ID NO:21 or 25). In certain embodiments, the CSAN comprises one, two, three, four, five, six, seven, or eight masked anti-CD3 sdAb-DHFR² recombinant protein monomer unit(s). In certain embodiments, the CSAN comprises one masked anti-CD3 sdAb-DHFR² recombinant protein monomer unit(s) (e.g., SEQ ID NO:21 or 25) and further comprises one, two, three, four, five, six, or seven additional masked anti-CD3 or anti-TAA sdAb-DHFR² recombinant protein monomer unit(s).

In certain embodiments, the CSAN comprises one or more masked anti-TAA sdAb-DHFR² monomer unit(s). In certain embodiments, the CSAN comprises one, two, three, four, five, six, seven, or eight masked anti-TAA sdAb-DHFR² monomer unit(s). In certain embodiments, the CSAN comprises one masked anti-TAA sdAb-DHFR² recombinant protein monomer unit(s) and further comprises one, two, three, four, five, six, or seven additional masked anti-TAA or anti-CD3 sdAb-DHFR² recombinant protein monomer unit(s).

In certain embodiments, the CSAN comprises one or more masked anti-PSMA sdAb-DHFR² monomer unit(s) (e.g., SEQ ID NO:23). In certain embodiments, the CSAN comprises one, two, three, four, five, six, or seven masked anti-PSMA sdAb-DHFR² monomer unit(s).

In certain embodiments, the CSAN comprises a) one or more unmasked anti-TAA sdAb-DHFR² monomer unit(s); and b) one or more masked anti-CD3 sdAb-DHFR² monomer unit(s) (e.g., SEQ ID NO:21 or 25). In certain embodiments, the ratio between a) and b) is 1:7, 1:3, 3:5, or 1:1. In certain embodiments, the ratio between a) and b) is 5:3, 3:1, or 7:1.

In certain embodiments, the CSAN comprises a) one or more unmasked anti-PSMA sdAb-DHFR² monomer unit(s) (e.g., SEQ ID NO:24); and b) one or more masked anti-CD3 sdAb-DHFR² monomer unit(s) (e.g., SEQ ID NO:21 or 25). In certain embodiments, the ratio between a) and b) is 1:7, 1:3, 3:5, or 1:1. In certain embodiments, the ratio between a) and b) is 5:3, 3:1, or 7:1.

In certain embodiments, the CSAN comprises a) one or more masked anti-TAA sdAb-DHFR² monomer unit(s) (e.g., SEQ ID NO:23); and b) one or more unmasked anti-CD3 sdAb-DHFR² monomer unit(s) (e.g., SEQ ID NO:22). In certain embodiments, the ratio between a) and b) is 1:7, 1:3, 3:5, or 1:1. In certain embodiments, the ratio between a) and b) is 5:3, 3:1, or 7:1.

In certain embodiments, the CSAN comprises a) one or more masked anti-PSMA sdAb-DHFR² monomer unit(s) (e.g., SEQ ID NO:23); and b) one or more unmasked anti-CD3 sdAb-DHFR² monomer unit(s) (e.g., SEQ ID NO:22). In certain embodiments, the ratio between a) and b) is 1:7, 1:3, 3:5, or 1:1. In certain embodiments, the ratio between a) and b) is 5:3, 3:1, or 7:1.

In certain embodiments, the CSAN comprises a) one or more masked anti-TAA sdAb-DHFR² monomer unit(s) (e.g., SEQ ID NO:23); and b) one or more anti-CD3 scFv-DHFR² monomer unit(s) (e.g., 1DD-anti-CD3 scFv, see SEQ ID NO:32). In certain embodiments, the ratio between a) and b) is 1:7, 1:3, 3:5, or 1:1. In certain embodiments, the ratio between a) and b) is 5:3, 3:1, or 7:1.

In certain embodiments, the CSAN comprises a) one or more masked anti-PSMA sdAb-DHFR² monomer unit(s) (e.g., SEQ ID NO:23); and b) one or more anti-CD3 scFv-DHFR² monomer unit(s) (e.g., 1DD-anti-CD3 scFv, see SEQ ID NO:32). In certain embodiments, the ratio between a) and b) is 1:7, 1:3, 3:5, or 1:1. In certain embodiments, the ratio between a) and b) is 5:3, 3:1, or 7:1.

In certain embodiments, the dual-masked CSAN comprises a) one or more masked anti-TAA sdAb-DHFR² monomer unit(s) (e.g., SEQ ID NO:23); and b) one or more masked anti-CD3 sdAb-DHFR² monomer unit(s) (e.g., SEQ ID NO:21 or 25). In certain embodiments, the ratio between a) and b) is 1:7, 1:3, 3:5, or 1:1. In certain embodiments, the ratio between a) and b) is 5:3, 3:1, or 7:1.

In certain embodiments, the dual-masked CSAN comprises a) one or more masked anti-PSMA sdAb-DHFR² monomer unit(s) (e.g., SEQ ID NO:23); and b) one or more masked anti-CD3 sdAb-DHFR² monomer unit(s) (e.g., SEQ ID NO:21 or 25). In certain embodiments, the ratio between a) and b) is 1:7, 1:3, 3:5, or 1:1. In certain embodiments, the ratio between a) and b) is 5:3, 3:1, or 7:1. In certain embodiments, the assembly (e.g., CSAN) or conjugate thereof may comprise a therapeutic agent or detectable agent. In certain embodiments, the CSAN may comprise one or more polypeptide monomer unit that is linked or conjugated to a therapeutic agent (e.g., toxic drug) or detectable agent (e.g., fluorescent agent). Thus, certain embodiments provide a conjugate comprising a CSAN as described herein.

TABLE 1

| SEQ | | |
|---|---|---|
| ID | | |
| NO: | Sequences | Comment |
| 1 | EVQLLEEVQLVESGGGLVQPGGSLRLSCAASGFTEDDYGMSWV | an exemplary anti- |
| | RQAPGKWLEWVSDISWNGGSTYYADSVKGRFTISRDNAENTLY | CD3 sdAb |
| | LQMNSLKPDDTAVYYCAKMGEGGWGANDYWGQGTQVTVSS | (CDR1, CDR2, and |
| | | CDR3 are underlined) |
| 2 | EVQLVESGGGLVQPGGSLTLSCAASREMISEYSMHWVRQAPGK | an exemplary anti- |
| | GLEWVSTINPAGTTDYAESVKGRFTISRDNAKNTLYLQMNSLK | PSMA sdAb |
| | PEDTAVYYCDGYGYRGQGTQVTVSS | |
| 3 | MADEIAKAQVARPGGDTIFGKIIRKEIPAKIIFEDDRSLAFHD | hHINT1 (126 aa in |
| | ISPQAPTHELVIPKKHISQISVAEDDDESLLGHLMIVGKKSAA | length) |
| | DLGLNKGYRMEVNEGSDGGQSVYHVHLHVLGGRQMHWPPG | |
| 31 | SNAMADEIAKAQVARPGGDTIFGKIIRKEIPAKIIFEDDRSLA | hHINT1 (129 aa in |
| | FHDISPQAPTHELVIPKKHISQISVAEDDDESLLGHLMIVGKK | length) |
| | SAADLGLNKGYRMEVNEGSDGGQSVYHVHLHVLGGRQMHWPPG | |
| 4 | GPLGVR | an exemplary MMP-2 |
| | | cleavage site |
| 5 | GGSGGGPLGVRGSGGEL | an exemplary Gly-Ser |
| | | linker with MMP-2 |
| | | cleavage site |
| | | (underlined) |
| 6 | MADEIAKAQVARPGGDTIFGKIIRKEIPAKIIFEDDRSLAFHD | an exemplary masked |
| | ISPQAPTHFLVIPKKHISQISVAEDDDESLLGHLMIVGKKSAA | sdAb sequence |
| | DLGLNKGYRMEVNEGSDGGQSVYHVHLHVLGGRQMHWPPGGGS | (hHINT1-anti-CD3): |
| | GGGPLGVRGSGGELEVQLLEEVQLVESGGGLVQPGGSLRLSCA | the masking hHINT1 |
| | ASGFTFDDYGMSWVRQAPGKWLEWVSDISWNGGSTYYADSVKG | sequence is bolded, |
| | RFTISRDNAENTLYLQMNSLKPDDTAVYYCAKMGEGGWGANDY | cleavage sequence is |
| | WGQGTQVTVSS | italicized (MMP-2 |
| | | cleavage site is |
| | | underlined) |
| 7 | MGHHHHHHMADEIAKAQVARPGGDTIFGKIIRKEIPAKIIFED | an exemplary masked |
| | DRSLAFHDISPQAPTHFLVIPKKHISQISVAEDDDESLLGHLM | sdAb sequence |
| | IVGKKSAADLGLNKGYRMEVNEGSDGGQSVYHVHLHVLGGRQM | (hHINT1-anti-CD3): |
| | HWPPGGGSGGGPLGVRGSGGELEVQLLEEVQLVESGGGLVQPG | the masking hHINT1 |
| | GSLRLSCAASGFTEDDYGMSWVRQAPGKWLEWVSDISWNGGST | sequence is bolded, |
| | YYADSVKGRFTISRDNAENTLYLQMNSLKPDDTAVYYCAKMGE | cleavage sequence is |
| | GGWGANDYWGQGTQVTVSS | italicized (MMP-2 |
| | | cleavage site is |
| | | underlined), the N- |
| | | terminal has a His |
| | | tag |
| 8 | MADEIAKAQVARPGGDTIFGKIIRKEIPAKIIFEDDRSLAFHD | an exemplary masked |
| | ISPQAPTHFLVIPKKHISQISVAEDDDESLLGHLMIVGKKSAA | sdAb sequence |
| | DLGLNKGYRMEVNEGSDGGQSVYHVHLHVLGGRQMHWPPGGGS | (hHINT1-anti-PSMA): |
| | GGGPLGVRGSGGELEVQLVESGGGLVQPGGSLTLSCAASREMI | the masking hHINT1 |
| | SEYSMHWVRQAPGKGLEWVSTINPAGTTDYAESVKGRFTISRD | sequence is bolded, |
| | NAKNTLYLQMNSLKPEDTAVYYCDGYGYRGQGTQVTVSS | cleavage sequence is |
| | | italicized (MMP-2 |
| | | cleavage site is |
| | | underlined) |
| 9 | MGHHHHHHMADEIAKAQVARPGGDTIFGKIIRKEIPAKIIFED | an exemplary masked |
| | DRSLAFHDISPQAPTHFLVIPKKHISQISVAEDDDESLLGHLM | sdAb sequence |
| | IVGKKSAADLGLNKGYRMEVNEGSDGGQSVYHVHLHVLGGRQM | (hHINT1-anti-PSMA): |
| | HWPPGGGSGGGPLGVRGSGGELEVQLVESGGGLVQPGGSLTLS | the masking hHINT1 |
| | CAASREMISEYSMHWVRQAPGKGLEWVSTINPAGTTDYAESVK | sequence is bolded, |
| | GRFTISRDNAKNTLYLQMNSLKPEDTAVYYCDGYGYRGQGTQV | cleavage sequence is |
| | TVSS | italicized (MMP-2 |
| | | cleavage site is |
| | | underlined), the N- |
| | | terminal has a His |
| | | tag |
| 10 | QDGNEEMGGITQTPYKVSISGTTVILT | an exemplary masking |
| | | sequence comprising |
| | | an epitope peptide |
| | | to which the anti- |
| | | CD3 sdAb (SEQ ID |
| | | NO: 1) bind |
| 11 | QDGNEEMGGITQTPYKVSISGTTVILTGGSGGGPLGVRGSGGE | an exemplary masked |
| | LEVQLLEEVQLVESGGGLVQPGGSLRLSCAASGFTEDDYGMSW | sdAb sequence |
| | VRQAPGKWLEWVSDISWNGGSTYYADSVKGRFTISRDNAENTL | (epitope peptide- |
| | YLQMNSLKPDDTAVYYCAKMGEGGWGANDYWGQGTQVTVSS | anti-CD3): the |
| | | masking sequence is |
| | | bolded, cleavage |
| | | sequence is |
| | | italicized (MMP-2 |
| | | cleavage site is |
| | | underlined) |
| 12 | MGHHHHHHQDGNEEMGGITQTPYKVSISGTTVILTGGSGGGPL | an exemplary masked |
| | GVRGSGGELEVQLLEEVQLVESGGGLVQPGGSLRLSCAASGFT | sdAb sequence |
| | FDDYGMSWVRQAPGKWLEWVSDISWNGGSTYYADSVKGRFTIS | (epitope peptide- |
| | RDNAENTLYLQMNSLKPDDTAVYYCAKMGEGGWGANDYWGQGT | anti-CD3): the |
| | QVTVSS | masking sequence is |
| | | bolded, cleavage |
| | | sequence is |
| | | italicized (MMP-2 |
| | | cleavage site is |
| | | underlined), the N- |
| | | terminal has a His |
| | | tag |
| 13 | GISLIAALAVDRVIGMENAMPWNLPADLAWFKRNTLNKPV | an exemplary DHFR |
| | IMGRHTWESIGRPLPGRKNIILSSQPGTDDRVTWVKSVDE | sequence |
| | AIAAAGDVPEIMVIGGGRVYEQFLPKAQKLYLTHIDAEVE | |
| | GDTHFPDYEPDDWESVESEFHDADAQNSHSYSFEILERR | |
| 36 | MISLIAALAVDRVIGMENAMPWNLPADLAWFKRNTLNKPV | an exemplary DHFR |
| | IMGRHTWESIGRPLPGRKNIILSSQPGTDDRVTWVKSVDE | sequence |
| | AIAAAGDVPEIMVIGGGRVYEQFLPKAQKLYLTHIDAEVE | |
| | GDTHFPDYEPDDWESVESEFHDADAQNSHSYSFEILERR | |
| 14 | GISLIAALAVDRVIGMENAMPWNLPADLAWFKRNTLNKPV | an exemplary DHFR2 |
| | IMGRHTWESIGRPLPGRKNIILSSQPGTDDRVTWVKSVDE | sequence (Glycine |
| | AIAAAGDVPEIMVIGGGRVYEQFLPKAQKLYLTHIDAEVE | linker is |
| | GDTHFPDYEPDDWESVFSEFHDADAQNSHSYSFEILERRG | underlined) |
| | GISLIAALAVDRVIGMENAMPWNLPADLAWFKRNTLNKPV | |
| | IMGRHTWESIGRPLPGRKNIILSSQPGTDDRVTWVKSVDE | |
| | AIAAAGDVPEIMVIGGGRVYEQFLPKAQKLYLTHIDAEVE | |
| | GDTHFPDYEPDDWESVESEFHDADAQNSHSYSFEILERR | |
| 37 | MISLIAALAVDRVIGMENAMPWNLPADLAWFKRNTLNKPV | an exemplary DHFR2 |
| | IMGRHTWESIGRPLPGRKNIILSSQPGTDDRVTWVKSVDE | sequence (Glycine |
| | AIAAAGDVPEIMVIGGGRVYEQFLPKAQKLYLTHIDAEVE | linker is |
| | GDTHFPDYEPDDWESVESEFHDADAQNSHSYSFEILERRG | underlined) |
| | MISLIAALAVDRVIGMENAMPWNLPADLAWFKRNTLNKPV | |
| | IMGRHTWESIGRPLPGRKNIILSSQPGTDDRVTWVKSVDE | |
| | AIAAAGDVPEIMVIGGGRVYEQFLPKAQKLYLTHIDAEVE | |
| | GDTHFPDYEPDDWESVESEFHDADAQNSHSYSFEILERR | |
| 15 | GGGGS | an exemplary Linker |
| | | sequence |
| 16 | GGGGSGGGGS | an exemplary Linker |
| | | sequence |
| 17 | GGSGG | an exemplary Linker |
| | | sequence |
| 18 | GGSGGGSGG | an exemplary Linker |
| | | sequence |
| 19 | GGGASGGGGSGGGGS | an exemplary Linker |
| | | sequence |
| 34 | GGSGGGSGGGSGG | an exemplary Linker |
| | | sequence |
| 20 | HHHHHH | His6 tag sequence |
| 21 | MGHHHHHHMADEIAKAQVARPGGDTIFGKIIRKEIPAKII | hHINT1-anti-CD3-1DD: |
| | FEDDRSLAFHDISPQAPTHFLVIPKKHISQISVAEDDDES | hHINT1-anti-CD3 (SEQ |
| | LLGHLMIVGKKSAADLGLNKGYRMEVNEGSDGGQSVYHVH | ID NO: 6) is operably |
| | LHVLGGRQMHWPPGGGSGGGPLGVRGSGGELEVQLLEEVQ | linked with DHFR2 |
| | LVESGGGLVQPGGSLRLSCAASGFTFDDYGMSWVRQAPGK | sequence (SEQ ID |
| | WLEWVSDISWNGGSTYYADSVKGRFTISRDNAENTLYLQM | NO: 14, underlined) by |
| | NSLKPDDTAVYYCAKMGEGGWGANDYWGQGTQVTVSSGGG | a linker |
| | ASGGGGSGGGGSGISLIAALAVDRVIGMENAMPWNLPADL | sequence (SEQ ID |
| | AWFKRNTLNKPVIMGRHTWESIGRPLPGRKNIILSSQPGT | NO: 19,bold/italic), |
| | DDRVTWVKSVDEAIAAAGDVPEIMVIGGGRVYEQFLPKAQ | the N terminal has |
| | KLYLTHIDAEVEGDTHEPDYEPDDWESVESEFHDADAQNS | an optional His6 tag |
| | HSYSFEILERRGGISLIAALAVDRVIGMENAMPWNLPADL | (SEQ ID NO: 20) |
| | AWFKRNTLNKPVIMGRHTWESIGRPLPGRKNIILSSQPGT | |
| | DDRVTWVKSVDEAIAAAGDVPEIMVIGGGRVYEQFLPKAQ | |
| | KLYLTHIDAEVEGDTHEPDYEPDDWESVESEFHDADAQNS | |
| | HSYSFEILERRGGGLKDYKDDDDK | |
| 22 | MGHHHHHHEVQLLEEVQLVESGGGLVQPGGSLRLSCAASG | anti-CD3-1DD: |
| | FTFDDYGMSWVRQAPGKWLEWVSDISWNGGSTYYADSVKG | unmasked anti-CD3 |
| | RFTISRDNAENTLYLQMNSLKPDDTAVYYCAKMGEGGWGA | (SEQ ID NO: 1) is |
| | NDYWGQGTQVTVSSGGGASGGGGSGGGGSGISLIAALAVD | operably linked with |
| | RVIGMENAMPWNLPADLAWFKRNTLNKPVIMGRHTWESIG | DHFR2 sequence (SEQ ID |
| | RPLPGRKNIILSSQPGTDDRVTWVKSVDEAIAAAGDVPEI | NO: 14, underlined) by |
| | MVIGGGRVYEQFLPKAQKLYLTHIDAEVEGDTHFPDYEPD | a linker |
| | DWESVESEFHDADAQNSHSYSFEILERRGGISLIAALAVD | sequence (SEQ ID |
| | RVIGMENAMPWNLPADLAWFKRNTLNKPVIMGRHTWESIG | NO: 19, bold/italic), |
| | RPLPGRKNIILSSQPGTDDRVTWVKSVDEAIAAAGDVPEI | the N terminal has |
| | MVIGGGRVYEQFLPKAQKLYLTHIDAEVEGDTHEPDYEPD | an optional His6 tag |
| | DWESVFSEFHDADAQNSHSYSFEILERRGGGLKDYKDDDD | (SEQ ID NO: 20) |
| | K | |
| 23 | MGHHHHHHMADEIAKAQVARPGGDTIFGKIIRKEIPAKII | hHINT1-anti-PSMA- |
| | FEDDRSLAFHDISPQAPTHFLVIPKKHISQISVAEDDDES | 1DD: |
| | LLGHLMIVGKKSAADLGLNKGYRMEVNEGSDGGQSVYHVH | hHINT1-anti-PSMA |
| | LHVLGGRQMHWPPGGGSGGGPLGVRGSGGELEVQLVESGG | (SEQ ID NO: 8) is |
| | GLVQPGGSLTLSCAASREMISEYSMHWVRQAPGKGLEWVS | operably linked with |
| | TINPAGTTDYAESVKGRFTISRDNAKNTLYLQMNSLKPED | DHFR2 sequence (SEQ ID |
| | TAVYYCDGYGYRGQGTQVTVSSGGGASGGGGSGGGGSGIS | NO: 14, underlined) by |
| | LIAALAVDRVIGMENAMPWNLPADLAWFKRNTLNKPVIMG | a linker |
| | RHTWESIGRPLPGRKNIILSSQPGTDDRVTWVKSVDEAIA | sequence (SEQ ID |
| | AAGDVPEIMVIGGGRVYEQFLPKAQKLYLTHIDAEVEGDT | NO: 19, bold/italic), |
| | HFPDYEPDDWESVESEFHDADAQNSHSYSFEILERRGGIS | the N terminal has |
| | LIAALAVDRVIGMENAMPWNLPADLAWFKRNTLNKPVIMG | an optional His6 tag |
| | RHTWESIGRPLPGRKNIILSSQPGTDDRVTWVKSVDEAIA | (SEQ ID NO: 20) |
| | AAGDVPEIMVIGGGRVYEQFLPKAQKLYLTHIDAEVEGDT | |
| | HFPDYEPDDWESVFSEFHDADAQNSHSYSFEILERRGGGL | |
| | KDYKDDDDK | |
| 24 | MGHHHHHHGGSGGGSGGEVQLVESGGGLVQPGGSLTLSCA | Anti-PSMA-1DD: |
| | ASRFMISEYSMHWVRQAPGKGLEWVSTINPAGTTDYAESV | unmasked anti-PSMA |
| | KGRFTISRDNAKNTLYLQMNSLKPEDTAVYYCDGYGYRGQ | (SEQ ID NO: 2) is |
| | GTQVTVSSGGGASGGGGSGGGGSGISLIAALAVDRVIGME | operably linked with |
| | NAMPWNLPADLAWFKRNTLNKPVIMGRHTWESIGRPLPGR | DHFR2 sequence (SEQ ID |
| | KNIILSSQPGTDDRVTWVKSVDEAIAAAGDVPEIMVIGGG | NO: 14, underlined) by |
| | RVYEQFLPKAQKLYLTHIDAEVEGDTHEPDYEPDDWESVE | a linker |
| | SEFHDADAQNSHSYSFEILERRGGISLIAALAVDRVIGME | sequence (SEQ ID |
| | NAMPWNLPADLAWFKRNTLNKPVIMGRHTWESIGRPLPGR | NO: 19,bold/italic), |
| | KNIILSSQPGTDDRVTWVKSVDEAIAAAGDVPEIMVIGGG | the N terminal has |
| | RVYEQFLPKAQKLYLTHIDAEVEGDTHEPDYEPDDWESVE | an optional His6 tag |
| | SEFHDADAQNSHSYSFEILERRGGGLKDYKDDDDK | (SEQ ID NO: 20)/ |
| | | linker (SEQ ID |
| | | NO: 18) |
| 25 | MGHHHHHHQDGNEEMGGITQTPYKVSISGTTVILTGGSGG | Peptide-xCD3-1DD: |
| | GPLGVRGSGGELEVQLLEEVQLVESGGGLVQPGGSLRLSC | peptide-anti-CD3 |
| | AASGFTFDDYGMSWVRQAPGKWLEWVSDISWNGGSTYYAD | (SEQ ID NO: 11) is |
| | SVKGRFTISRDNAENTLYLQMNSLKPDDTAVYYCAKMGEG | operably linked with |
| | GWGANDYWGQGTQVTVSSGGGASGGGGSGGGGSGISLIAA | DHFR2 sequence (SEQ ID |
| | LAVDRVIGMENAMPWNLPADLAWFKRNTLNKPVIMGRHTW | NO: 14, underlined) by |
| | ESIGRPLPGRKNIILSSQPGTDDRVTWVKSVDEAIAAAGD | a linker |
| | VPEIMVIGGGRVYEQFLPKAQKLYLTHIDAEVEGDTHEPD | sequence (SEQ ID |
| | YEPDDWESVESEFHDADAQNSHSYSFEILERRGGISLIAA | NO: 19,bold/italic), |
| | LAVDRVIGMENAMPWNLPADLAWFKRNTLNKPVIMGRHTW | the N terminal has |
| | ESIGRPLPGRKNIILSSQPGTDDRVTWVKSVDEAIAAAGD | an optional His6 tag |
| | VPEIMVIGGGRVYEQFLPKAQKLYLTHIDAEVEGDTHEPD | (SEQ ID NO: 20) |
| | YEPDDWESVESEFHDADAQNSHSYSFEILERRGGGLKDYK | |
| | DDDDK | |
| 32 | MGHHHHHHGGSGGGSGGGSGGMISLIAALAVDRVIGMENA | 1DD-Anti-CD3 scFv |
| | MPWNLPADLAWFKRNTLNKPVIMGRHTWESIGRPLPGRKN | DHFR2 sequence (SEQ ID |
| | IILSSQPGTDDRVTWVKSVDEAIAAAGDVPEIMVIGGGRV | NO: 37, underlined) is |
| | YEQFLPKAQKLYLTHIDAEVEGDTHFPDYEPDDWESVESE | operably linked with |
| | FHDADAQNSHSYSFEILERRGMISLIAALAVDRVIGMENA | Anti-CD3 ScFv (SEQ |
| | MPWNLPADLAWFKRNTLNKPVIMGRHTWESIGRPLPGRKN | ID NO: 35) by a |
| | IILSSQPGTDDRVTWVKSVDEAIAAAGDVPEIMVIGGGRV | linker sequence (SEQ |
| | YEQFLPKAQKLYLTHIDAEVEGDTHEPDYEPDDWESVESE | ID |
| | FHDADAQNSHSYSFEILERRGGSGGGSGGGSGGDIQMTQT | NO: 34, bold/italic), |
| | TSSLSASLGDRVTISCRASQDIRNYLNWYQQKPDGTVKLL | the N terminal of |
| | IYYTSRLHSGVPSKFSGSGSGTDYSLTISNLEQEDIATYF | this construct has |
| | CQQGNTLPWTFAGGTKLEIKRGGGGSGGGGSGGGGSGGRE | an optional His6 tag |
| | VQLQQSGPELVKPGASMKISCKASGYSFTGYTMNWVKQSH | (SEQ ID NO: 20)/ |
| | GKNLEWMGLINPYKGVSTYNQKFKDKATLTVDKSSSTAYM | linker (SEQ ID |
| | ELLSLTSEDSAVYYCARSGYYGDSDWYFDVWGAGTTVTVS | NO: 34) |
| | S | |
| 35 | DIQMTQTTSSLSASLGDRVTISCRASQDIRNYLNWYQQKP | Anti-CD3 scFv |
| | DGTVKLLIYYTSRLHSGVPSKFSGSGSGTDYSLTISNLEQ | |
| | EDIATYFCQQGNTLPWTFAGGTKLEIKRGGGGSGGGGSGG | |
| | GGSGGREVQLQQSGPELVKPGASMKISCKASGYSFTGYTM | |
| | NWVKQSHGKNLEWMGLINPYKGVSTYNQKFKDKATLTVDK | |
| | SSSTAYMELLSLTSEDSAVYYCARSGYYGDSDWYFDVWGA | |
| | GTTVTVSS | |
| 33 | ATGGGACACCATCACCATCACCACGGTGGTTCAGGTGGTGGTT | an exemplary DNA |
| | CAGGAGGTGGATCTGGAGGTATGATCAGTCTGATTGCGGCGTT | sequence encoding |
| | AGCGGTAGATCGCGTGATTGGTATGGAAAACGCCATGCCGTGG | 1DD-anti-CD3 ScFv |
| | AACCTGCCTGCCGATCTCGCCTGGTTTAAACGCAACACCTTAA | |
| | ATAAACCCGTGATTATGGGCCGCCATACCTGGGAATCAATCGG | |
| | TCGTCCGTTGCCAGGACGCAAAAATATTATCCTCAGCAGTCAA | |
| | CCGGGTACGGACGATCGCGTAACGTGGGTGAAGTCGGTGGATG | |
| | AAGCCATCGCGGCGGCTGGTGACGTACCAGAAATCATGGTGAT | |
| | TGGCGGCGGTCGCGTTTATGAACAGTTCTTGCCAAAAGCGCAA | |
| | AAACTGTATCTGACGCATATCGACGCAGAAGTGGAAGGCGACA | |
| | CCCATTTCCCGGATTACGAGCCGGATGACTGGGAATCGGTATT | |
| | CAGTGAATTCCACGATGCTGATGCGCAGAACTCTCACAGCTAT | |
| | AGCTTTGAGATTCTGGAGCGGCGGGGCATGATCTCTCTGATCG | |
| | CGGCTCTTGCTGTTGATCGCGTTATCGGCATGGAGAATGCAAT | |
| | GCCTTGGAACTTGCCAGCGGACCTTGCTTGGTTCAAGCGTAAT | |
| | ACATTGAATAAACCTGTAATCATGGGTCGCCATACGTGGGAGT | |
| | CGATCGGCCGCCCTCTGCCCGGCCGCAAAAACATCATTCTGAG | |
| | CTCTCAACCAGGTACTGATGACCGTGTTACGTGGGTTAAAAGT | |
| | GTAGACGAAGCCATTGCAGCTGCGGGTGATGTACCCGAGATTA | |
| | TGGTAATCGGAGGGGGGCGTGTATACGAACAGTTCTTGCCCAA | |
| | GGCGCAAAAGTTATATTTGACGCATATTGATGCCGAAGTCGAA | |
| | GGCGATACACATTTTCCGGATTACGAGCCTGATGACTGGGAAT | |
| | CGGTTTTTTCCGAGTTTCATGACGCGGATGCCCAAAACTCTCA | |
| | CAGTTATTCTTTTGAGATTCTTGAACGTCGCGGGGGCAGTGGA | |
| | GGTGGCAGTGGAGGTGGGTCTGGGGGCGACATTCAGATGACGC | |
| | AGACGACATCGAGCTTATCCGCAAGCCTGGGGGATCGCGTTAC | |
| | GATCTCTTGTCGTGCATCCCAAGACATCCGCAACTACTTAAAC | |
| | TGGTATCAACAAAAACCGGATGGAACCGTGAAATTGCTTATCT | |
| | ACTATACTTCGCGCCTGCACTCGGGAGTACCCTCAAAGTTTAG | |
| | CGGCTCCGGGAGTGGTACAGACTATAGTCTTACCATTTCCAAT | |
| | CTTGAACAGGAAGACATTGCAACGTACTTTTGCCAACAAGGGA | |
| | ATACTCTTCCTTGGACTTTTGCGGGAGGGACGAAACTTGAGAT | |
| | TAAACGCGGCGGGGGAGGATCAGGGGGTGGGGGCTCAGGAGGC | |
| | GGAGGTTCCGGCGGGCGCGAGGTACAATTACAGCAGTCCGGGC | |
| | CGGAGCTTGTTAAGCCGGGAGCGAGCATGAAAATTTCATGTAA | |
| | GGCAAGTGGCTATAGCTTCACTGGTTATACCATGAACTGGGTT | |
| | AAGCAATCGCACGGAAAGAATCTGGAGTGGATGGGTTTGATTA | |
| | ACCCCTATAAGGGGGTTTCGACTTACAATCAAAAGTTTAAAGA | |
| | CAAAGCCACACTGACTGTTGATAAGAGTTCTTCGACCGCATAC | |
| | ATGGAATTGCTGTCACTGACCAGTGAGGATTCGGCGGTGTACT | |
| | ATTGTGCACGCAGCGGTTATTATGGAGATTCCGACTGGTACTT | |
| | CGATGTCTGGGGAGCTGGTACAACGGTCACCGTTAGTTCA | |
| 26 | ATGGGCCACCATCACCATCATCATATGGCAGATGAGATTGCCA | an exemplary DNA |
| | AGGCTCAGGTCGCTCGGCCTGGTGGCGACACGATCTTTGGGAA | sequence encoding |
| | GATCATCCGCAAGGAAATACCAGCCAAAATCATTTTTGAGGAT | hHINT1-anti-CD3-1DD |
| | GACCGGAGCCTTGCTTTCCATGACATTTCCCCTCAAGCACCAA | |
| | CACATTTTCTGGTGATACCCAAGAAACATATATCCCAGATTTC | |
| | TGTGGCAGAAGATGATGATGAAAGTCTTCTTGGACACTTAATG | |
| | ATTGTTGGCAAGAAAAGCGCTGCTGATCTGGGCCTGAATAAGG | |
| | GTTATCGAATGGAAGTGAATGAAGGTTCAGATGGTGGACAGTC | |
| | TGTCTATCACGTTCATCTCCATGTTCTTGGAGGTCGGCAAATG | |
| | CATTGGCCTCCTGGTGGAGGTTCAGGTGGTGGACCGCTGGGAG | |
| | TGCGCGGCAGCGGAGGCGAGCTCGAGGTGCAGCTGCTCGAGGA | |
| | GGTGCAGCTGGTGGAGTCTGGGGGAGGCTTGGTGCAGCCTGGG | |
| | GGGTCTCTGAGACTCTCCTGTGCAGCCTCTGGATTCACTTTTG | |
| | ATGATTATGGCATGAGCTGGGTCCGACAGGCTCCAGGGAAGTG | |
| | GCTGGAGTGGGTCTCAGATATTAGCTGGAATGGTGGTAGCACA | |
| | TACTATGCAGACTCCGTGAAGGGCCGGTTCACCATCTCCAGAG | |
| | ACAACGCCGAGAACACGCTGTATCTGCAAATGAACAGCCTGAA | |
| | ACCTGACGACACGGCCGTGTATTACTGTGCAAAAATGGGTGAA | |
| | GGGGGATGGGGTGCAAATGACTACTGGGGCCAGGGGACCCAGG | |
| | TCACCGTCTCCTCCGGAGGCGGAGCTAGCGGTGGAGGCGGCTC | |
| | AGGGGGAGGTGGATCCGGAATCTCGTTGATTGCGGCATTAGCG | |
| | GTCGACCGCGTTATCGGAATGGAAAACGCGATGCCCTGGAATT | |
| | TACCTGCTGACCTTGCTTGGTTCAAGCGTAACACTTTAAACAA | |
| | GCCGGTGATTATGGGACGCCACACGTGGGAATCCATCGGCCGC | |
| | CCTCTGCCGGGACGTAAGAACATCATTCTTTCAAGCCAACCAG | |
| | GAACCGACGATCGCGTGACGTGGGTCAAGAGTGTCGACGAAGC | |
| | AATCGCGGCCGCGGGAGACGTCCCGGAAATCATGGTCATCGGA | |
| | GGAGGACGTGTCTATGAGCAGTTTTTGCCTAAGGCGCAGAAGC | |
| | TGTACTTAACCCATATCGACGCAGAGGTGGAGGGCGACACACA | |
| | CTTCCCCGATTACGAGCCCGATGATTGGGAGTCAGTGTTCTCA | |
| | GAATTTCACGACGCGGATGCGCAGAACTCTCACTCTTATAGTT | |
| | TCGAGATTTTGGAGCGCCGCGGTGGAATTAGTCTTATCGCTGC | |
| | GTTGGCAGTCGATCGCGTAATCGGTATGGAGAATGCTATGCCT | |
| | TGGAACCTTCCCGCAGACTTGGCCTGGTTCAAACGCAATACTT | |
| | TAAATAAACCTGTGATTATGGGCCGTCATACTTGGGAGTCGAT | |
| | CGGGCGTCCTTTGCCCGGACGCAAGAATATCATTTTGAGTTCC | |
| | CAACCGGGCACCGATGATCGTGTTACGTGGGTTAAGAGTGTGG | |
| | ACGAAGCTATCGCCGCTGCTGGGGACGTACCCGAAATTATGGT | |
| | TATTGGGGGTGGACGCGTATATGAGCAATTTCTGCCGAAAGCC | |
| | CAAAAACTTTATCTTACCCACATTGATGCCGAAGTGGAAGGCG | |
| | ATACGCATTTCCCGGACTATGAGCCGGATGATTGGGAATCAGT | |
| | GTTTAGCGAGTTTCACGATGCAGACGCTCAGAACAGTCATTCA | |
| | TACTCGTTTGAGATTTTAGAGCGCCGTGGAGGTGGCCTTAAGG | |
| | ATTACAAGGACGACGATGACAAGTAA | |
| 27 | ATGGGCCACCATCACCATCATCATGAGGTGCAGCTGCTCGAGG | an exemplary DNA |
| | AGGTGCAGCTGGTGGAGTCTGGGGGAGGCTTGGTGCAGCCTGG | sequence encoding |
| | GGGGTCTCTGAGACTCTCCTGTGCAGCCTCTGGATTCACTTTT | anti-CD3-1DD |
| | GATGATTATGGCATGAGCTGGGTCCGACAGGCTCCAGGGAAGT | |
| | GGCTGGAGTGGGTCTCAGATATTAGCTGGAATGGTGGTAGCAC | |
| | ATACTATGCAGACTCCGTGAAGGGCCGGTTCACCATCTCCAGA | |
| | GACAACGCCGAGAACACGCTGTATCTGCAAATGAACAGCCTGA | |
| | AACCTGACGACACGGCCGTGTATTACTGTGCAAAAATGGGTGA | |
| | AGGGGGATGGGGTGCAAATGACTACTGGGGCCAGGGGACCCAG | |
| | GTCACCGTCTCCTCCGGAGGCGGAGCTAGCGGTGGAGGCGGCT | |
| | CAGGGGGAGGTGGATCCGGAATCTCGTTGATTGCGGCATTAGC | |
| | GGTCGACCGCGTTATCGGAATGGAAAACGCGATGCCCTGGAAT | |
| | TTACCTGCTGACCTTGCTTGGTTCAAGCGTAACACTTTAAACA | |
| | AGCCGGTGATTATGGGACGCCACACGTGGGAATCCATCGGCCG | |
| | CCCTCTGCCGGGACGTAAGAACATCATTCTTTCAAGCCAACCA | |
| | GGAACCGACGATCGCGTGACGTGGGTCAAGAGTGTCGACGAAG | |
| | CAATCGCGGCCGCGGGAGACGTCCCGGAAATCATGGTCATCGG | |
| | AGGAGGACGTGTCTATGAGCAGTTTTTGCCTAAGGCGCAGAAG | |
| | CTGTACTTAACCCATATCGACGCAGAGGTGGAGGGCGACACAC | |
| | ACTTCCCCGATTACGAGCCCGATGATTGGGAGTCAGTGTTCTC | |
| | AGAATTTCACGACGCGGATGCGCAGAACTCTCACTCTTATAGT | |
| | TTCGAGATTTTGGAGCGCCGCGGTGGAATTAGTCTTATCGCTG | |
| | CGTTGGCAGTCGATCGCGTAATCGGTATGGAGAATGCTATGCC | |
| | TTGGAACCTTCCCGCAGACTTGGCCTGGTTCAAACGCAATACT | |
| | TTAAATAAACCTGTGATTATGGGCCGTCATACTTGGGAGTCGA | |
| | TCGGGCGTCCTTTGCCCGGACGCAAGAATATCATTTTGAGTTC | |
| | CCAACCGGGCACCGATGATCGTGTTACGTGGGTTAAGAGTGTG | |
| | GACGAAGCTATCGCCGCTGCTGGGGACGTACCCGAAATTATGG | |
| | TTATTGGGGGTGGACGCGTATATGAGCAATTTCTGCCGAAAGC | |
| | CCAAAAACTTTATCTTACCCACATTGATGCCGAAGTGGAAGGC | |
| | GATACGCATTTCCCGGACTATGAGCCGGATGATTGGGAATCAG | |
| | TGTTTAGCGAGTTTCACGATGCAGACGCTCAGAACAGTCATTC | |
| | ATACTCGTTTGAGATTTTAGAGCGCCGTGGAGGTGGCCTTAAG | |
| | GATTACAAGGACGACGATGACAAGTAA | |
| 28 | ATGGGCCACCATCACCATCATCATATGGCAGATGAGATTGCCA | an exemplary DNA |
| | AGGCTCAGGTCGCTCGGCCTGGTGGCGACACGATCTTTGGGAA | sequence encoding |
| | GATCATCCGCAAGGAAATACCAGCCAAAATCATTTTTGAGGAT | hHINT1-anti-PSMA-1DD |
| | GACCGGAGCCTTGCTTTCCATGACATTTCCCCTCAAGCACCAA | |
| | CACATTTTCTGGTGATACCCAAGAAACATATATCCCAGATTTC | |
| | TGTGGCAGAAGATGATGATGAAAGTCTTCTTGGACACTTAATG | |
| | ATTGTTGGCAAGAAAAGCGCTGCTGATCTGGGCCTGAATAAGG | |
| | GTTATCGAATGGAAGTGAATGAAGGTTCAGATGGTGGACAGTC | |
| | TGTCTATCACGTTCATCTCCATGTTCTTGGAGGTCGGCAAATG | |
| | CATTGGCCTCCTGGTGGAGGTTCAGGTGGTGGACCGCTGGGAG | |
| | TGCGCGGTAGCGGAGGCGAGCTCGAAGTGCAATTAGTAGAGAG | |
| | TGGCGGGGGGCTTGTTCAGCCCGGAGGTAGCTTGACTCTTTCC | |
| | TGCGCGGCCAGCCGTTTTATGATTTCCGAGTATTCTATGCACT | |
| | GGGTGCGTCAGGCGCCCGGCAAGGGACTGGAATGGGTCAGTAC | |
| | GATTAATCCCGCCGGTACGACCGATTATGCGGAGAGCGTAAAA | |
| | GGCCGTTTCACTATCTCTCGCGATAACGCCAAAAATACCTTAT | |
| | ATTTGCAAATGAATTCCCTTAAACCAGAAGATACGGCTGTCTA | |
| | CTATTGCGACGGCTACGGATACCGTGGCCAAGGGACCCAAGTC | |
| | ACGGTTTCATCAGGAGGCGGAGCTAGCGGTGGAGGCGGCTCAG | |
| | GGGGAGGTGGATCCGGAATCTCGTTGATTGCGGCATTAGCGGT | |
| | CGACCGCGTTATCGGAATGGAAAACGCGATGCCCTGGAATTTA | |
| | CCTGCTGACCTTGCTTGGTTCAAGCGTAACACTTTAAACAAGC | |
| | CGGTGATTATGGGACGCCACACGTGGGAATCCATCGGCCGCCC | |
| | TCTGCCGGGACGTAAGAACATCATTCTTTCAAGCCAACCAGGA | |
| | ACCGACGATCGCGTGACGTGGGTCAAGAGTGTCGACGAAGCAA | |
| | TCGCGGCCGCGGGAGACGTCCCGGAAATCATGGTCATCGGAGG | |
| | AGGACGTGTCTATGAGCAGTTTTTGCCTAAGGCGCAGAAGCTG | |
| | TACTTAACCCATATCGACGCAGAGGTGGAGGGCGACACACACT | |
| | TCCCCGATTACGAGCCCGATGATTGGGAGTCAGTGTTCTCAGA | |
| | ATTTCACGACGCGGATGCGCAGAACTCTCACTCTTATAGTTTC | |
| | GAGATTTTGGAGCGCCGCGGTGGAATTAGTCTTATCGCTGCGT | |
| | TGGCAGTCGATCGCGTAATCGGTATGGAGAATGCTATGCCTTG | |
| | GAACCTTCCCGCAGACTTGGCCTGGTTCAAACGCAATACTTTA | |
| | AATAAACCTGTGATTATGGGCCGTCATACTTGGGAGTCGATCG | |
| | GGCGTCCTTTGCCCGGACGCAAGAATATCATTTTGAGTTCCCA | |
| | ACCGGGCACCGATGATCGTGTTACGTGGGTTAAGAGTGTGGAC | |
| | GAAGCTATCGCCGCTGCTGGGGACGTACCCGAAATTATGGTTA | |
| | TTGGGGGTGGACGCGTATATGAGCAATTTCTGCCGAAAGCCCA | |
| | AAAACTTTATCTTACCCACATTGATGCCGAAGTGGAAGGCGAT | |
| | ACGCATTTCCCGGACTATGAGCCGGATGATTGGGAATCAGTGT | |
| | TTAGCGAGTTTCACGATGCAGACGCTCAGAACAGTCATTCATA | |
| | CTCGTTTGAGATTTTAGAGCGCCGTGGAGGTGGCCTTAAGGAT | |
| | TACAAGGACGACGATGACAAGTAA | |
| 29 | atgGGCCACCATCACCATCATCATGGCGGTAGCGGAGGCGGTT | an exemplary DNA |
| | CAGGTGGTGAAGTGCAATTAGTAGAGAGTGGCGGGGGGCTTGT | sequence encoding |
| | TCAGCCCGGAGGTAGCTTGACTCTTTCCTGCGCGGCCAGCCGT | anti-PSMA-1DD |
| | TTTATGATTTCCGAGTATTCTATGCACTGGGTGCGTCAGGCGC | |
| | CCGGCAAGGGACTGGAATGGGTCAGTACGATTAATCCCGCCGG | |
| | TACGACCGATTATGCGGAGAGCGTAAAAGGCCGTTTCACTATC | |
| | TCTCGCGATAACGCCAAAAATACCTTATATTTGCAAATGAATT | |
| | CCCTTAAACCAGAAGATACGGCTGTCTACTATTGCGACGGCTA | |
| | CGGATACCGTGGCCAAGGGACCCAAGTCACGGTTTCATCAGGA | |
| | GGCGGAGCTAGCGGTGGAGGCGGCTCAGGGGGAGGTGGATCCG | |
| | GAATCTCGTTGATTGCGGCATTAGCGGTCGACCGCGTTATCGG | |
| | AATGGAAAACGCGATGCCCTGGAATTTACCTGCTGACCTTGCT | |
| | TGGTTCAAGCGTAACACTTTAAACAAGCCGGTGATTATGGGAC | |
| | GCCACACGTGGGAATCCATCGGCCGCCCTCTGCCGGGACGTAA | |
| | GAACATCATTCTTTCAAGCCAACCAGGAACCGACGATCGCGTG | |
| | ACGTGGGTCAAGAGTGTCGACGAAGCAATCGCGGCCGCGGGAG | |
| | ACGTCCCGGAAATCATGGTCATCGGAGGAGGACGTGTCTATGA | |
| | GCAGTTTTTGCCTAAGGCGCAGAAGCTGTACTTAACCCATATC | |
| | GACGCAGAGGTGGAGGGCGACACACACTTCCCCGATTACGAGC | |
| | CCGATGATTGGGAGTCAGTGTTCTCAGAATTTCACGACGCGGA | |
| | TGCGCAGAACTCTCACTCTTATAGTTTCGAGATTTTGGAGCGC | |
| | CGCGGTGGAATTAGTCTTATCGCTGCGTTGGCAGTCGATCGCG | |
| | TAATCGGTATGGAGAATGCTATGCCTTGGAACCTTCCCGCAGA | |
| | CTTGGCCTGGTTCAAACGCAATACTTTAAATAAACCTGTGATT | |
| | ATGGGCCGTCATACTTGGGAGTCGATCGGGCGTCCTTTGCCCG | |
| | GACGCAAGAATATCATTTTGAGTTCCCAACCGGGCACCGATGA | |
| | TCGTGTTACGTGGGTTAAGAGTGTGGACGAAGCTATCGCCGCT | |
| | GCTGGGGACGTACCCGAAATTATGGTTATTGGGGGTGGACGCG | |
| | TATATGAGCAATTTCTGCCGAAAGCCCAAAAACTTTATCTTAC | |
| | CCACATTGATGCCGAAGTGGAAGGCGATACGCATTTCCCGGAC | |
| | TATGAGCCGGATGATTGGGAATCAGTGTTTAGCGAGTTTCACG | |
| | ATGCAGACGCTCAGAACAGTCATTCATACTCGTTTGAGATTTT | |
| | AGAGCGCCGTGGAGGTGGCCTTAAGGATTACAAGGACGACGAT | |
| | GACAAGTAA | |
| 30 | ATGGGCCACCATCACCATCATCATCAGGATGGCAACGAAGAAA | an exemplary DNA |
| | TGGGCGGCATTACCCAGACCCCGTATAAAGTGAGCATTAGCGG | sequence encoding |
| | CACCACCGTGATTCTGACCGGAGGTTCAGGTGGTGGACCGCTG | Peptide-αCD3-1DD |
| | GGAGTGCGCGGCAGCGGAGGCGAGCTCGAGGTGCAGCTGCTCG | |
| | AGGAGGTGCAGCTGGTGGAGTCTGGGGGAGGCTTGGTGCAGCC | |
| | TGGGGGGTCTCTGAGACTCTCCTGTGCAGCCTCTGGATTCACT | |
| | TTTGATGATTATGGCATGAGCTGGGTCCGACAGGCTCCAGGGA | |
| | AGTGGCTGGAGTGGGTCTCAGATATTAGCTGGAATGGTGGTAG | |
| | CACATACTATGCAGACTCCGTGAAGGGCCGGTTCACCATCTCC | |
| | AGAGACAACGCCGAGAACACGCTGTATCTGCAAATGAACAGCC | |
| | TGAAACCTGACGACACGGCCGTGTATTACTGTGCAAAAATGGG | |
| | TGAAGGGGGATGGGGTGCAAATGACTACTGGGGCCAGGGGACC | |
| | CAGGTCACCGTCTCCTCCGGAGGCGGAGCTAGCGGTGGAGGCG | |
| | GCTCAGGGGGAGGTGGATCCGGAATCTCGTTGATTGCGGCATT | |
| | AGCGGTCGACCGCGTTATCGGAATGGAAAACGCGATGCCCTGG | |
| | AATTTACCTGCTGACCTTGCTTGGTTCAAGCGTAACACTTTAA | |
| | ACAAGCCGGTGATTATGGGACGCCACACGTGGGAATCCATCGG | |
| | CCGCCCTCTGCCGGGACGTAAGAACATCATTCTTTCAAGCCAA | |
| | CCAGGAACCGACGATCGCGTGACGTGGGTCAAGAGTGTCGACG | |
| | AAGCAATCGCGGCCGCGGGAGACGTCCCGGAAATCATGGTCAT | |
| | CGGAGGAGGACGTGTCTATGAGCAGTTTTTGCCTAAGGCGCAG | |
| | AAGCTGTACTTAACCCATATCGACGCAGAGGTGGAGGGCGACA | |
| | CACACTTCCCCGATTACGAGCCCGATGATTGGGAGTCAGTGTT | |
| | CTCAGAATTTCACGACGCGGATGCGCAGAACTCTCACTCTTAT | |
| | AGTTTCGAGATTTTGGAGCGCCGCGGTGGAATTAGTCTTATCG | |
| | CTGCGTTGGCAGTCGATCGCGTAATCGGTATGGAGAATGCTAT | |
| | GCCTTGGAACCTTCCCGCAGACTTGGCCTGGTTCAAACGCAAT | |
| | ACTTTAAATAAACCTGTGATTATGGGCCGTCATACTTGGGAGT | |
| | CGATCGGGCGTCCTTTGCCCGGACGCAAGAATATCATTTTGAG | |
| | TTCCCAACCGGGCACCGATGATCGTGTTACGTGGGTTAAGAGT | |
| | GTGGACGAAGCTATCGCCGCTGCTGGGGACGTACCCGAAATTA | |
| | TGGTTATTGGGGGTGGACGCGTATATGAGCAATTTCTGCCGAA | |
| | AGCCCAAAAACTTTATCTTACCCACATTGATGCCGAAGTGGAA | |
| | GGCGATACGCATTTCCCGGACTATGAGCCGGATGATTGGGAAT | |
| | CAGTGTTTAGCGAGTTTCACGATGCAGACGCTCAGAACAGTCA | |
| | TTCATACTCGTTTGAGATTTTAGAGCGCCGTGGAGGTGGCCTT | |
| | AAGGATTACAAGGACGACGATGACAAGTAA | |

Certain embodiments provide a nucleic acid as provided herein. For example, certain embodiments provide a polynucleotide comprising a nucleic acid sequence having at least about 70%, 75%, 80%, 85%, 90%, 95%, 96%, 97%, 98%, 99% or 100% sequence identity to a sequence described herein (e.g., any one of SEQ ID NOs: 26-30).

Certain embodiments also provide expression cassettes and vectors comprising such nucleic acids. For example, certain embodiments provide an expression cassette comprising a nucleic acid as described herein and a promoter. Certain embodiments also provide a vector comprising such an expression cassette.

Certain embodiments of the invention provide a cell comprising a nucleic acid, expression cassette or vector as described herein. In certain embodiments, the cell expresses a recombinant protein described herein as a cell membrane anchored protein on the cell surface.

Certain embodiments of the invention provide a method for treating cancer in an animal in need of, comprising administering a therapeutically effective amount of a recombinant protein (e.g., a masked sdAb or other fusion protein described herein) as described herein to the animal (e.g., a mammal such as human). In certain embodiments, the tumor is a solid tumor. In certain embodiments, the cancer is prostate cancer, lung cancer, hepatic cancer, head and neck cancer, pancreatic cancer, brain cancer, colon cancer, bile duct cancer, or breast cancer.

Certain embodiments of the invention provide a method for treating cancer in an animal in need of, comprising administering a therapeutically effective amount of an assembly (e.g., a C SAN) as described herein to the animal (e.g., a mammal such as human). In certain embodiments, the cancer is prostate cancer, lung cancer, hepatic cancer, head and neck cancer, pancreatic cancer, brain cancer, colon cancer, bile duct cancer, or breast cancer.

Certain embodiments of the invention provide a method comprising contacting a recombinant protein (e.g., a masked sdAb) as described herein with an immune cell (e.g., T cell) or a tumor cell.

Certain embodiments of the invention provide a method comprising contacting an assembly (e.g., a CSAN) as described herein with an immune cell (e.g., T cell) or a tumor cell.

In certain embodiments, the contacting is in vitro. In certain embodiments, the contacting is in vivo. In certain embodiments, the immune cell (e.g., T cell) or the tumor cell is located in a solid tumor. In certain embodiments, the immune cell is a CD8⁺ cytotoxic T cell. In certain embodiments, the tumor cell expresses or overexpresses a TAA (e.g., HER2, EGFR, EpCAM, or PSMA). In certain embodiments, the tumor cell expresses a protease such as a MMP (e.g., MMP2).

Certain embodiments of the invention provide a method comprising contacting a recombinant protein or an assembly (e.g., a CSAN) as described herein with a tumor in an animal having cancer. In certain embodiments, the tumor comprises cells that express a protease such as a MMP (e.g., MMP2).

Certain embodiments of the invention provide a method comprising contacting a recombinant protein or an assembly (e.g., a CSAN) as described herein with a hHINT1 substrate that could be activated by hHINT1 to emit detectable signal (e.g., fluorescent signal). In certain embodiments, the contacting is in vitro. In certain embodiments, the contacting is in vivo. In certain embodiment, the method further comprises detecting the signal. In certain embodiment, the hHINT1 substrate is Tryptamine-5′-adenosine phosphoramidate.

### Administration

The proteins, polypeptides and assemblies (e.g., a CSAN), or conjugates thereof (e.g., a conjugate comprising a recombinant protein or CSAN as described herein), described herein can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present proteins, polypeptides and assemblies, or conjugate thereof, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the proteins, polypeptides and assemblies, or conjugate thereof, may be combined with one or more excipients and used in the form of ingestible tablets, buccal tablets, troches, capsules, elixirs, suspensions, syrups, wafers, and the like. Such compositions and preparations should contain at least 0.1% of protein, polypeptide or conjugate. The percentage of the compositions and preparations may, of course, be varied and may conveniently be between about 2 to about 60% of the weight of a given unit dosage form. The amount of the protein, polypeptide, assembly, or conjugate thereof in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the proteins, polypeptides or conjugates, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the proteins, polypeptides or conjugates may be incorporated into sustained-release preparations and devices.

The proteins, polypeptides or assemblies, or conjugate thereof, may also be administered intravenously or intraperitoneally by infusion or injection. Solutions of the proteins, polypeptides, assemblies, or conjugates thereof, can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the proteins, polypeptides or conjugates which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the proteins, polypeptides or assemblies, or a conjugate thereof, in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the proteins, polypeptides or conjugates plus any additional desired ingredient present in the previously sterile-filtered solutions.

For topical administration, the present proteins, polypeptides or assemblies, or a conjugate thereof, may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present proteins, polypeptides or conjugates can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the proteins, polypeptides, assemblies, or a conjugate thereof to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the proteins, polypeptides and assemblies, or a conjugate thereof, can be determined by comparing their in vitro activity, and in vivo activity in animal models. Methods for the extrapolation of effective dosages in mice, and other animals, to humans are known to the art; for example, see U.S. Pat. No. 4,938,949.

The amount of the proteins, polypeptides or assemblies, or conjugate thereof, required for use in treatment will vary with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into several discrete loosely spaced administrations.

### Certain Embodiments

Embodiment 1. A protein or polypeptide as described herein.

Embodiment 2. A protein or polypeptide comprising an amino acid sequence having at least about 70%, 75%, 80%, 85%, 90%, 95%, 96%, 97%, 98%, 99% or 100% sequence identity to a sequence described herein.

Embodiment 3. A nucleic acid encoding a polypeptide as described herein.

Embodiment 4. A nucleic acid comprising a sequence having at least about 70%, 75%, 80%, 85%, 90%, 95%, 96%, 97%, 98%, 99% or 100% sequence identity to a sequence described herein.

Embodiment 5. An expression cassette comprising a nucleic acid sequence as described herein, and optionally a promoter operably linked to the nucleic acid

Embodiment 6. A vector comprising an expression cassette, wherein the expression cassette comprises a nucleic acid sequence as described herein and a promoter operably linked to the nucleic acid.

Embodiment 7. A protein comprising a human histidine nucleotide triad binding protein 1 (hHINT1) polypeptide linked through a polypeptide linker to a nanobody, or an antigen binding fragment thereof.

Embodiment 8. The protein of embodiment 7, wherein the hHINT1 polypeptide comprises an amino acid sequence having at least about 70%, 75%, 80%, 85%, 90%, 95%, 96%, 97%, 98%, 99% or 100% sequence identity to a hHINT1 sequence described herein.

Embodiment 9. A protein comprising a human CD3ε derived peptide linked through a polypeptide linker to a nanobody, or an antigen binding fragment thereof.

Embodiment 10. The protein of embodiment 9, wherein the human CD3ε derived peptide comprises an amino acid sequence having at least about 70%, 75%, 80%, 85%, 90%, 95%, 96%, 97%, 98%, 99% or 100% sequence identity to a human CD3ε peptide sequence described herein.

Embodiment 11. The protein of any one of embodiments 7-10, wherein the linker is a flexible polypeptide linker.

Embodiment 12. The protein of any one of embodiments 7-11, wherein the linker is cleavable polypeptide linker that is capable of being cleaved by a proteolytic enzyme expressed within a solid tumor.

Embodiment 13. The protein of embodiment 12, wherein the cleavable polypeptide linker comprises a polypeptide sequence disclosed herein (e.g., MMP-2 substrate sequence GPLGVR (SEQ ID NO: 4)).

Embodiment 14. The protein of embodiment 12, wherein the proteolytic enzyme is MMP-2.

Embodiment 15. The protein of any one of embodiments 7-14, wherein the nanobody is anti-CD3 nanobody, or an antigen binding fragment thereof.

Embodiment 16. A masked nanobody, or an antigen binding fragment thereof, as described herein.

Embodiment 17. A CSAN conjugate comprising a polypeptide or protein as described herein.

Embodiment 18. A pharmaceutical composition comprising a polypeptide, protein or conjugate as described herein and a pharmaceutically acceptable excipient.

Embodiment 19. A method for treating or preventing cancer in an animal comprising administering a therapeutically effective amount of a protein or conjugate as described herein to the animal.

Embodiment 20. A method as described herein for preparing a masked nanobody.

### Certain Definitions

As used herein the term “plurality” refers to 2 or more.

As used herein, the term “specifically binds” when referring to the interaction between two molecules (e.g., between a sdAb and its target antigen; or between a sdAb and epitope polypeptide sequence in a masking polypeptide), refers to a binding reaction whereby a given molecule binds to its target with greater affinity, greater avidity, and/or greater duration than it binds to a structurally different target. For example, in certain embodiments, a given sdAb has at least 5-fold, 10-fold, 50-fold, 100-fold, 1,000-fold, 10,000-fold, or greater affinity for a specific target epitope polypeptide sequence as compared to an unrelated target when assayed under the same affinity assay conditions.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucl. Acids Res., 19:508; Ohtsuka et al. (1985) JBC, 260:2605; Rossolini et al. (1994) Mol. Cell. Probes, 8:91. A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

By “portion” or “fragment,” as it relates to a nucleic acid molecule, sequence or segment of the invention, when it is linked to other sequences for expression, is meant a sequence having at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention.

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆) alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein).

The terms “protein,” “peptide” and “polypeptide” are used interchangeably herein. Polypeptide sequences specifically recited herein are written with the amino terminus on the left and the carboxy terminus on the right.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

“Wild-type” refers to the normal gene, or organism found in nature without any known mutation.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Recombinant DNA molecule” is a combination of DNA sequences that are joined together using recombinant DNA technology and procedures used to join together DNA sequences as described, for example, in Sambrook and Russell, Molecular Cloning: A Laboratory Manual, Cold Spring Harbor, NY: Cold Spring Harbor Laboratory Press (3^{rd} edition, 2001).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

The term “gene” is used broadly to refer to any segment of nucleic acid associated with a biological function. Genes include coding sequences and/or the regulatory sequences required for their expression. For example, gene refers to a nucleic acid fragment that expresses mRNA, functional RNA, or a specific protein, including its regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

A “vector” is defined to include, inter alia, any viral vector, plasmid, cosmid, phage or binary vector in double or single stranded linear or circular form which may or may not be self-transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al. (1995) Mol. Biotech. 3:225).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. “Operably-linked” also refers to the association of two moieties (e.g., chemical moieties and/or protein domains) that are linked directly or indirectly, for example, via fusion as a recombinant protein and/or conjugation via covalent or noncovalent bonding.

“Expression” refers to the transcription and/or translation in a cell of an endogenous gene, transgene, as well as the transcription and stable accumulation of sense (mRNA) or functional RNA. In the case of antisense constructs, expression may refer to the transcription of the anti sense DNA only. Expression may also refer to the production of protein.

“Transcription stop fragment” refers to nucleotide sequences that contain one or more regulatory signals, such as polyadenylation signal sequences, capable of terminating transcription. Examples of transcription stop fragments are known to the art.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

The following terms are used to describe the sequence relationships between two or more sequences (e.g., nucleic acids, polynucleotides or polypeptides): (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA, gene sequence or peptide sequence, or the complete cDNA, gene sequence or peptide sequence.

(b) As used herein, “comparison window” makes reference to a contiguous and specified segment of a sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988) CABIOS, 4:11; the local homology algorithm of Smith et al. (1981) Adv. Appl. Math. 2:482; the homology alignment algorithm of Needleman and Wunsch, (1970) JMB, 48:443; the search-for-similarity-method of Pearson and Lipman, (1988) Proc. Natl. Acad. Sci. USA, 85:2444; the algorithm of Karlin and Altschul, (1990) Proc. Natl. Acad. Sci. USA, 87:2264, modified as in Karlin and Altschul, (1993) Proc. Natl. Acad. Sci. USA, 90:5873.

Computer implementations of these mathematical algorithms can be utilized for comparison of sequences to determine sequence identity. Such implementations include, but are not limited to: CLUSTAL in the PC/Gene program (available from Intelligenetics, Mountain View, California); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wisconsin, USA). Alignments using these programs can be performed using the default parameters. The CLUSTAL program is well described by Higgins et al. (1988) Gene 73:237; Higgins et al. (1989) CABIOS 5:151; Corpet et al. (1988) Nucl. Acids Res. 16:10881; Huang et al. (1992) CABIOS 8:155; and Pearson et al. (1994) Meth. Mol. Biol. 24:307. The ALIGN program is based on the algorithm of Myers and Miller, supra. The BLAST programs of Altschul et al. (1990) JMB, 215:403; Nucl. Acids Res., (1990), are based on the algorithm of Karlin and Altschul supra.

Software for performing BLAST analyses is publicly available through the National Center for Biotechnology Information (available on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of sequences for determination of percent sequence identity to another sequence may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

(d) As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, at least 95%.

Another indication that nucleotide sequences are substantially identical is if two molecules hybridize to each other under stringent conditions (see below). Generally, stringent conditions are selected to be about 5° C. lower than the thermal melting point (T m) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (T m) is the temperature (under defined ionic strength and pH) at which 50% of the target sequence hybridizes to a perfectly matched probe. Specificity is typically the function of post-hybridization washes, the critical factors being the ionic strength and temperature of the final wash solution.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel (1985) Proc. Natl. Acad. Sci. USA 82:488; Kunkel et al. (1987) Meth. Enzymol. 154:367; U.S. Pat. No. 4,873,192; Walker and Gaastra (1983) Techniques in Mol. Biol. (MacMillan Publishing Co., and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass naturally occurring proteins as well as variations and modified forms thereof. Such variants will continue to possess the desired activity. In certain embodiments, the deletions, insertions, and substitutions of the polypeptide sequence encompassed herein may not produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” “transduced” and “recombinant” refer to a host cell or organism into which a heterologous nucleic acid molecule has been introduced. The nucleic acid molecule can be stably integrated into the genome generally known in the art and are disclosed in Sambrook and Russell, supra. See also Innis et al., PCR Protocols, Academic Press (1995); and Gelfand, PCR Strategies, Academic Press (1995); and Innis and Gelfand, PCR Methods Manual, Academic Press (1999). Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

As used herein, the term “derived” or “directed to” with respect to a nucleotide molecule means that the molecule has complementary sequence identity to a particular molecule of interest.

The terms “treat” and “treatment” refer to both therapeutic treatment and prophylactic or preventative measures, wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a protein, polypeptide, assembly, or conjugate of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the polypeptide, protein, assembly, or conjugate may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells.

The invention will now be illustrated by the following non-limiting Examples.

### Example 1. Construction and Characterization of Pro-Chemically Self-Assembled Nanorings (Pro-CSANs) for Cancer Immunotherapy

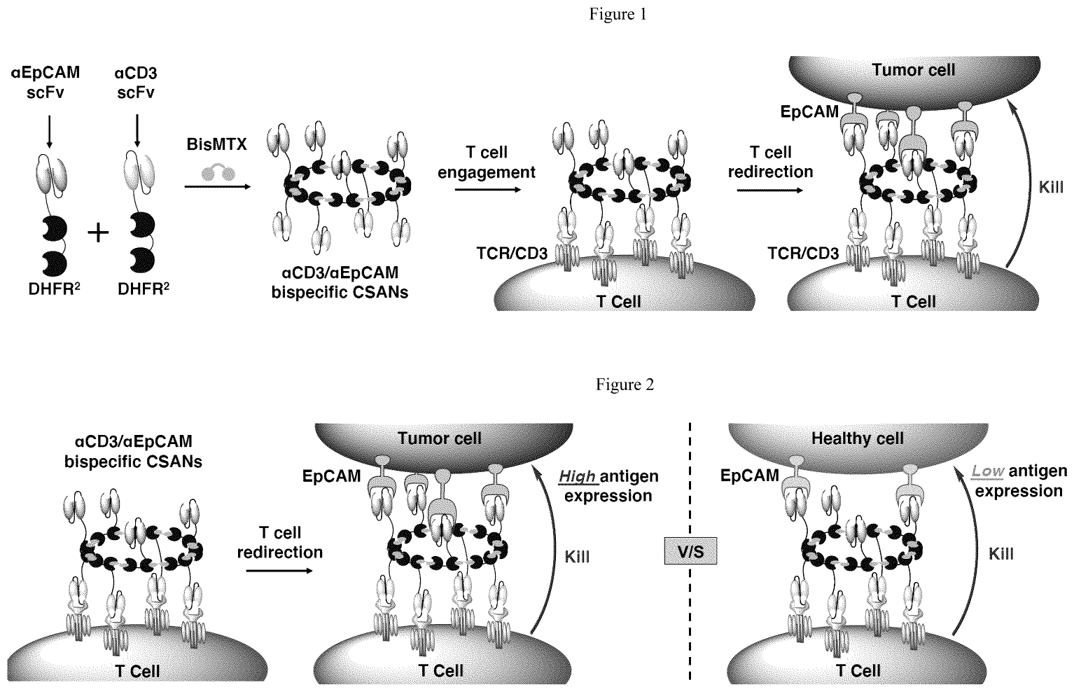

We have previously developed a non-genetic platform to re-direct human T cells to combat solid tumors. This approach is based on fusing a tumor-targeting protein or a T cell-targeting anti-CD3 protein to a dimer of Escherichia coli (E. coli) dihydrofolate reductase (DHFR²). Incubating a mixture of tumor-targeting and T cell-targeting DHFR² fusion proteins with a chemical dimerizer, bis-methotrexate, leads to the spontaneous assembly of bispecific, multivalent nanorings, or CSANs (Shen, J. et al. J. Am. Chem. Soc. 2015, 137, 10108-10111., and Petersburg, J. R. ACS Nano. 2018, 12, 6563-6576). These bispecific CSANs bind to CD3⁺ T cells and re-direct them to target and destroy cancer cells (e.g., see FIG. 1).

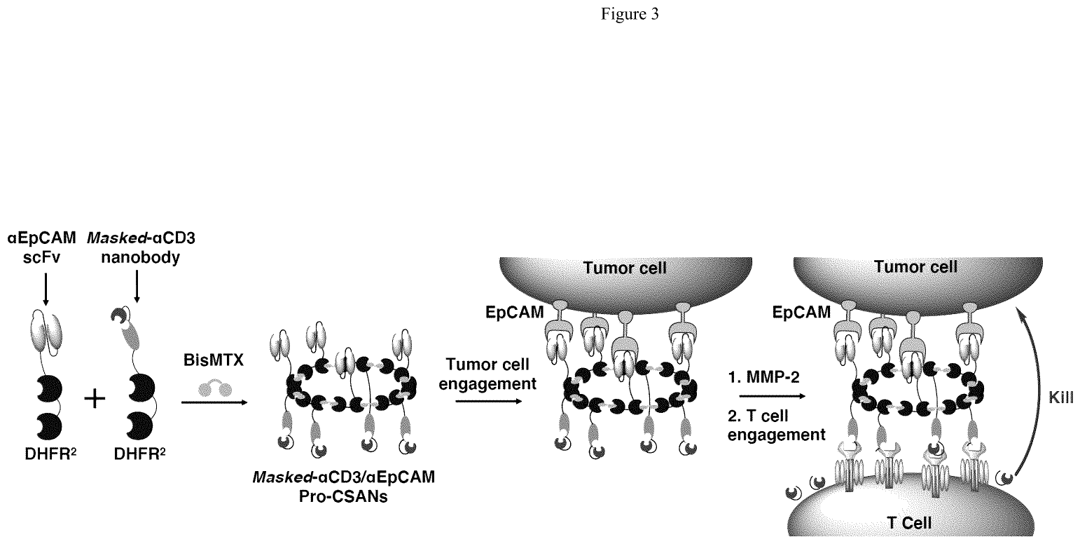

Tumor-associated antigen targeting relies on differential antigen expression between cancer and healthy cells. The high affinity and avidity of the multivalent bispecific CSANs may potentially target healthy tissue with low antigen expression, thereby potentially exhibiting “on-target, off-tumor” toxicity (e.g., see FIG. 2). Matrix metalloproteinases are overexpressed in a variety of solid tumors. Therefore, to improve tumor specificity, we hypothesized herein that masking the anti-CD3 moiety in our CSANs with a peptide or a protein, through a matrix metalloproteinase-2 (MMP-2) sensitive linker will prevent engagement and activation of T cells outside the tumor microenvironment (TME). Once the CSANs enter the TME, cleavage by MMP-2 will lead to unmasking of the anti-CD3 moiety, followed by engagement and activation of T cells, ultimately causing targeted cancer cell lysis (e.g., see FIG. 3).

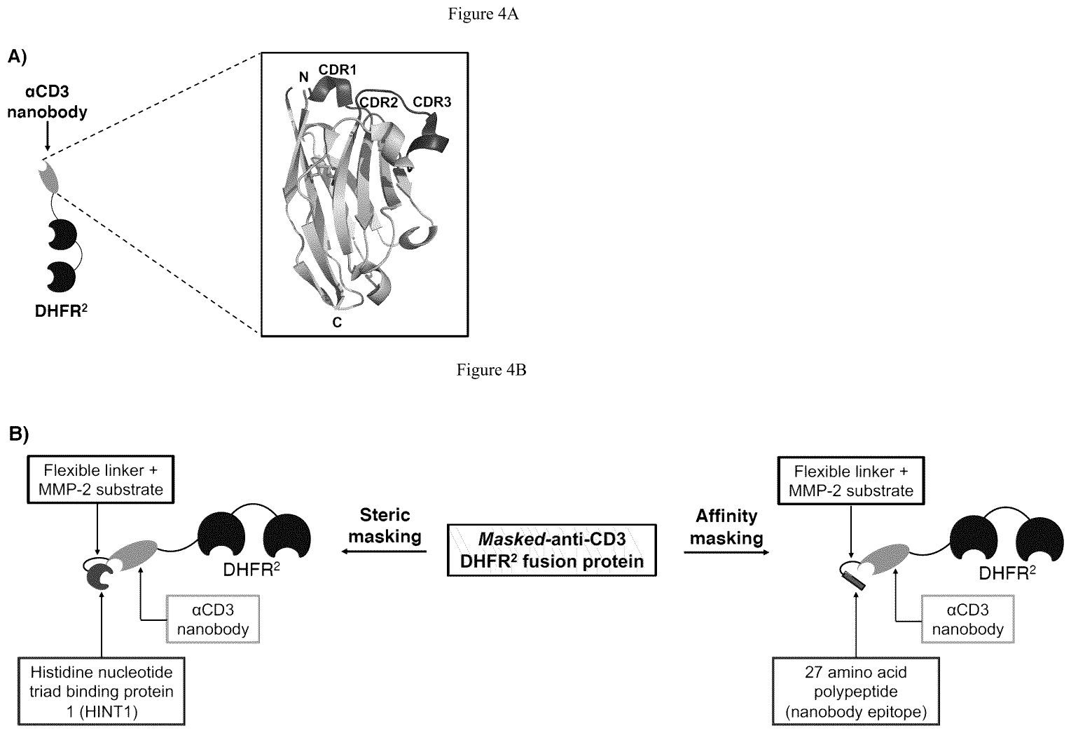

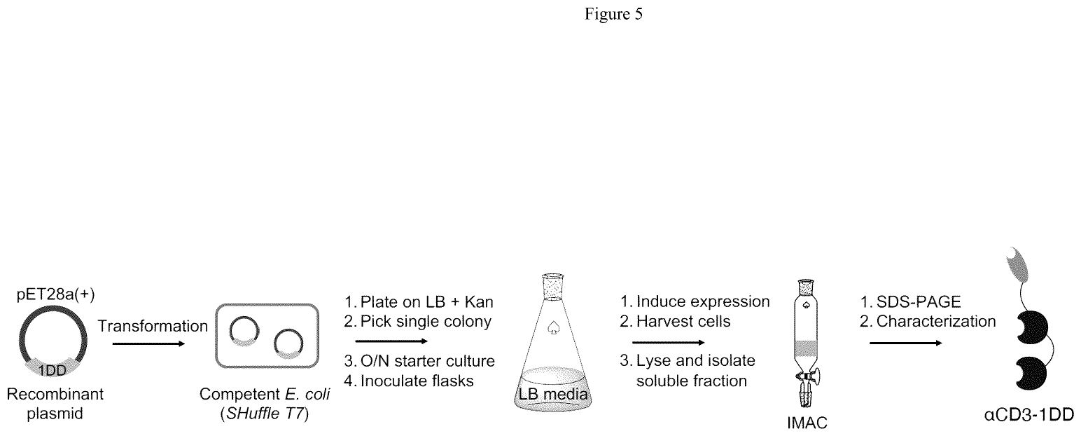

To this end, we generated an anti-CD3-DHFR fusion protein containing an anti-CD3 nanobody (e.g., see FIG. 4A). To mask the anti-CD3 nanobody, the first design embodiment involved genetically fusing a 27 amino acid polypeptide to the nanobody's N-terminus via an MMP-2 sensitive linker (e.g., see FIG. 4B right side). In the second design embodiment, the polypeptide was replaced with human histidine triad nucleotide binding protein 1, which will potentially serve as a steric mask (e.g., see FIG. 4B left side). The anti-CD3 and masked-anti-CD3-DHFR² fusion proteins were successfully expressed and purified from E. coli. Characterizations of these fusion proteins are described herein.

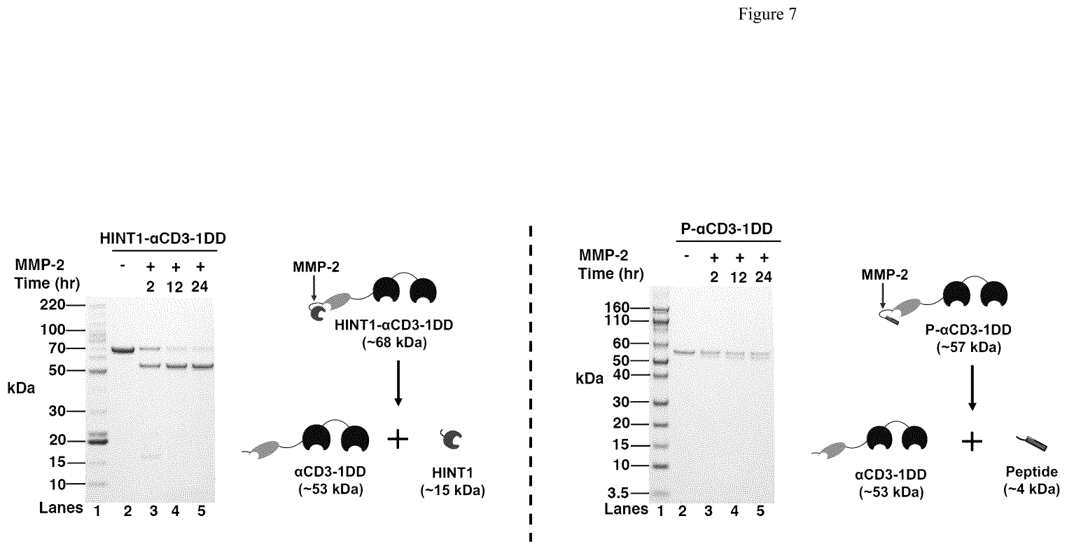

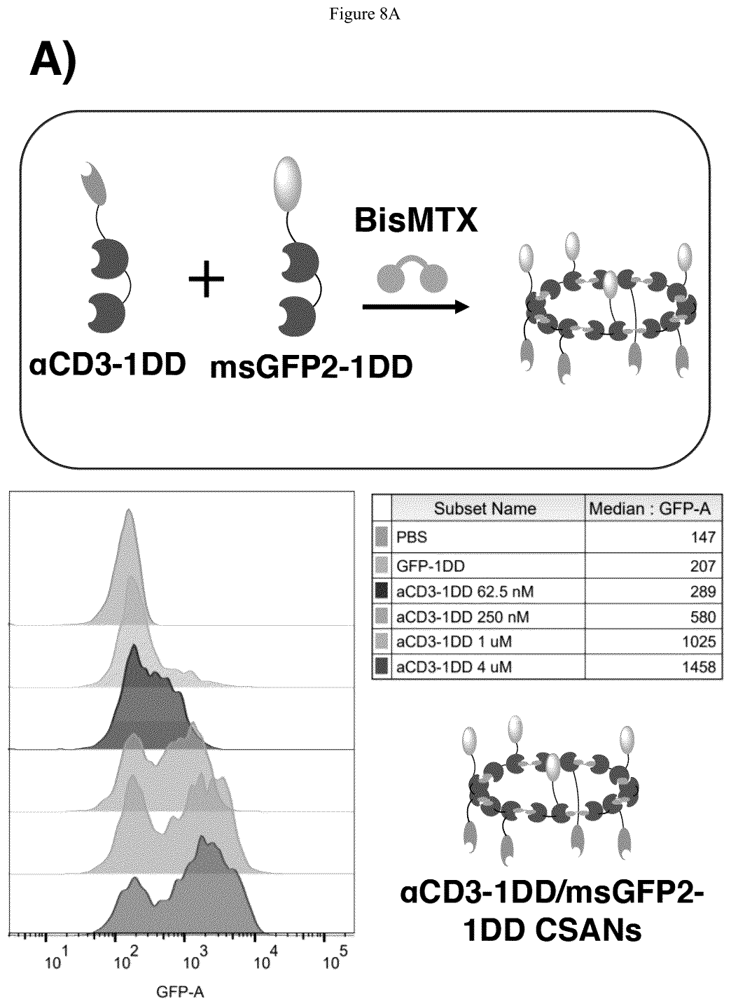

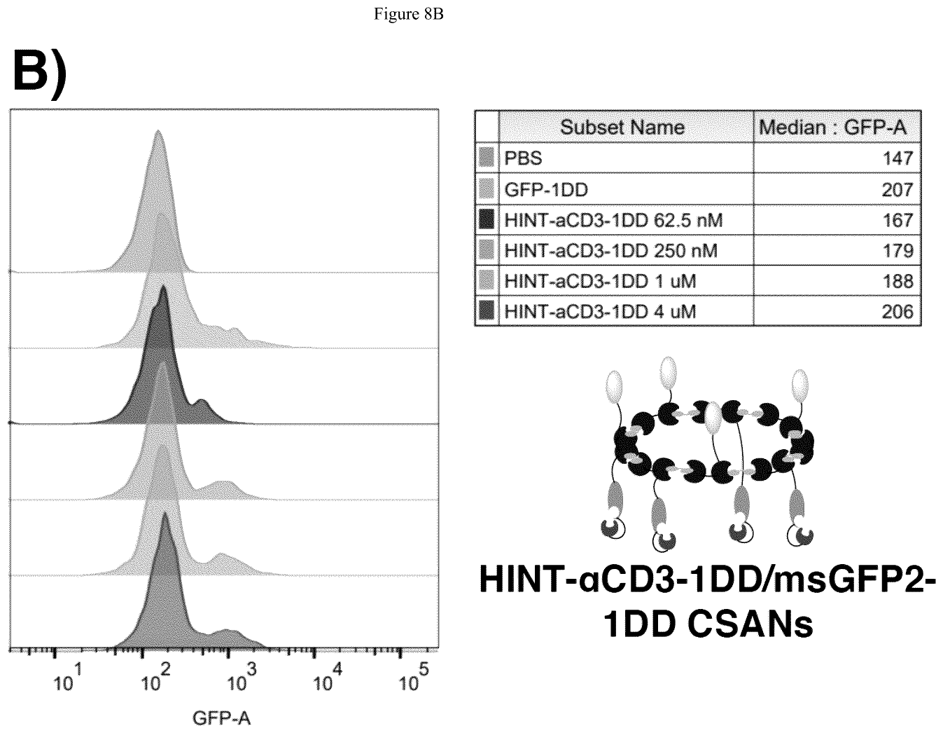

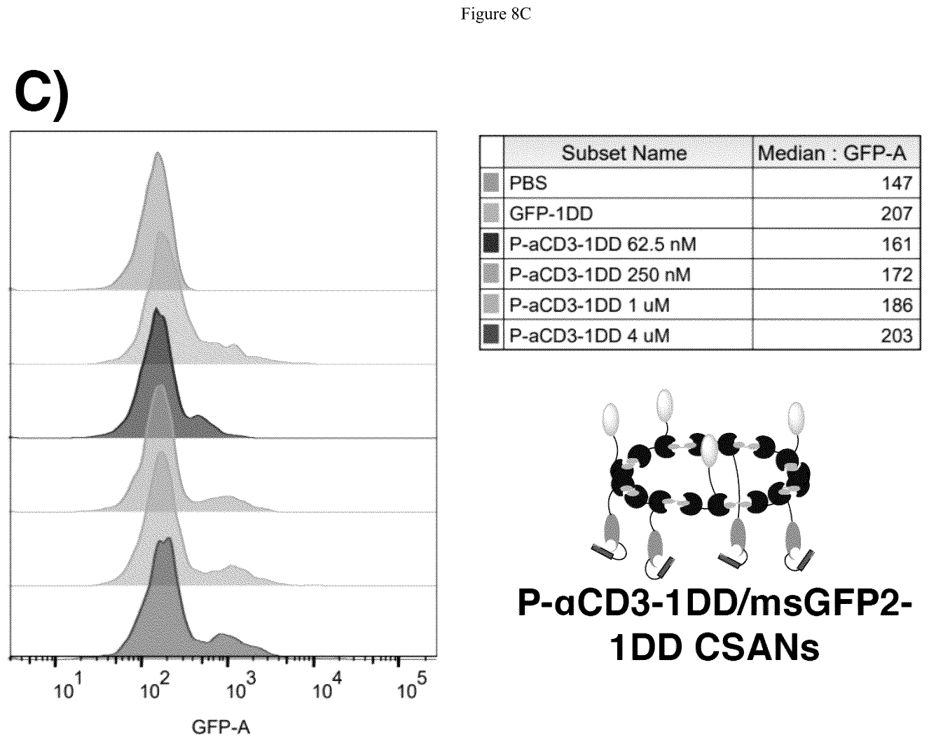

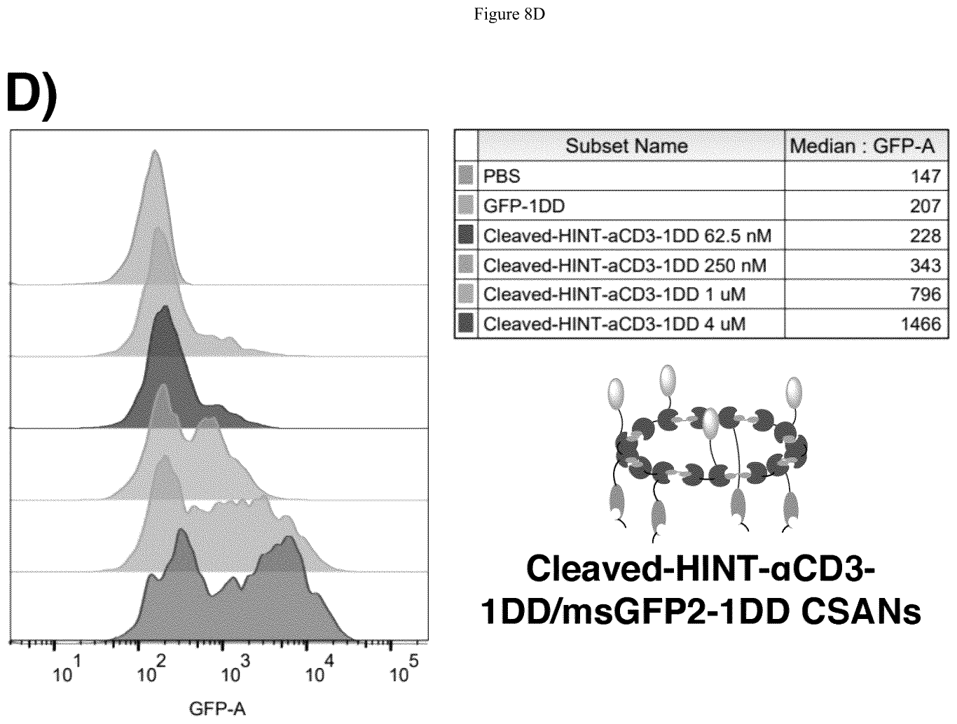

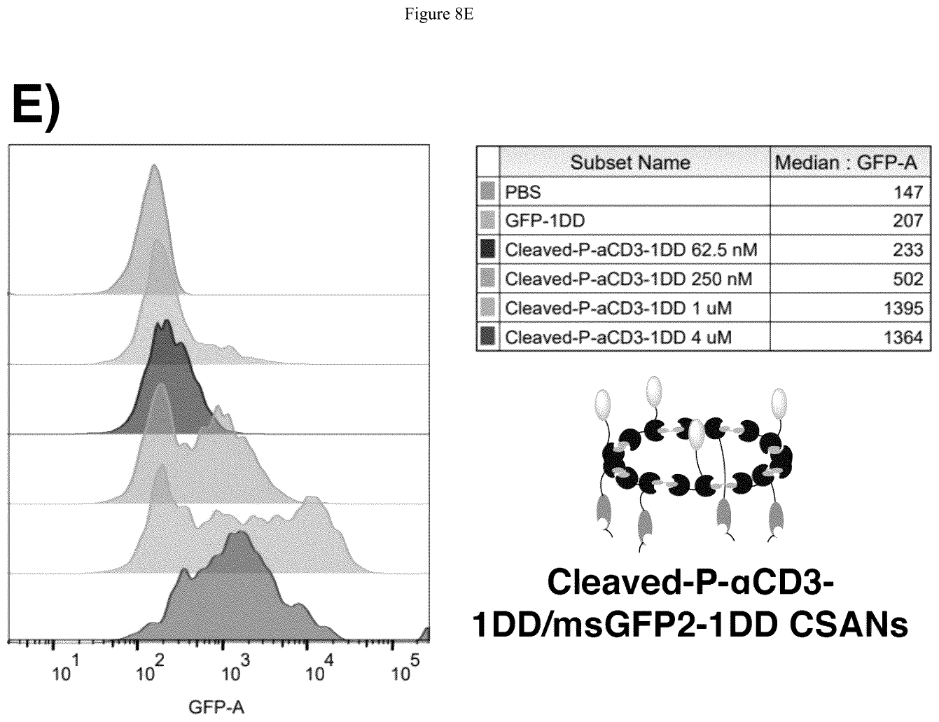

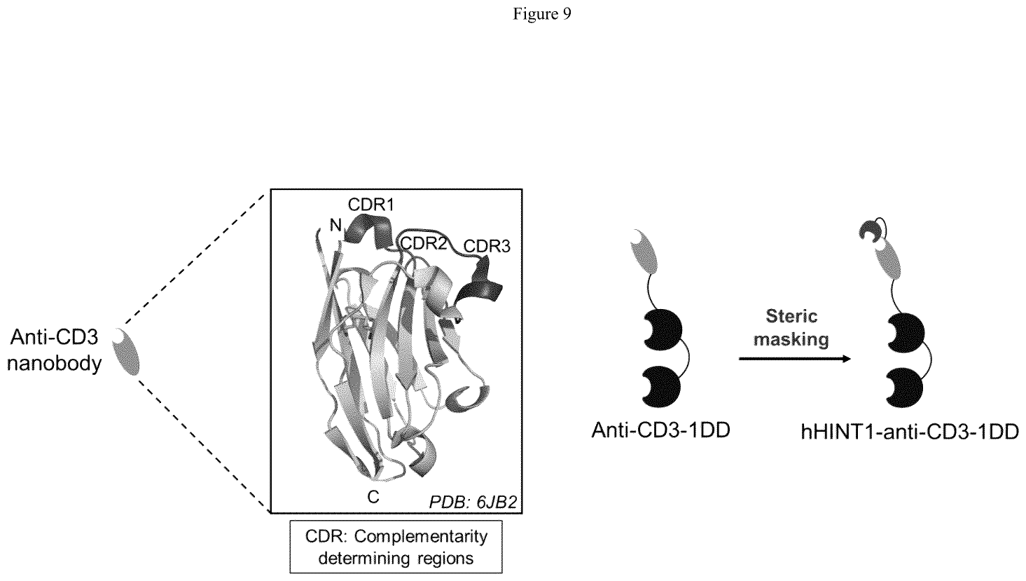

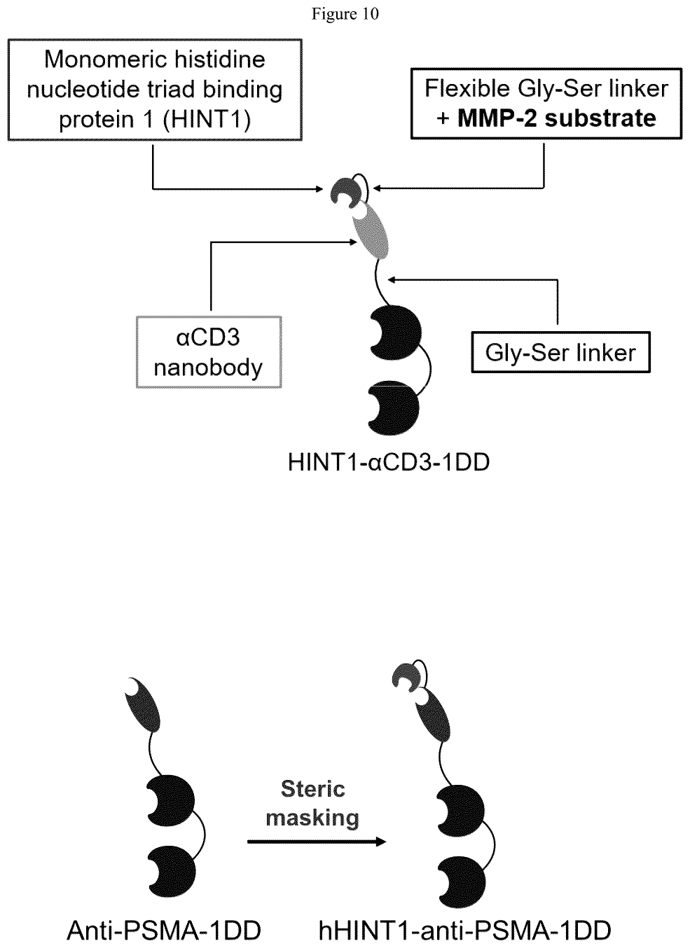

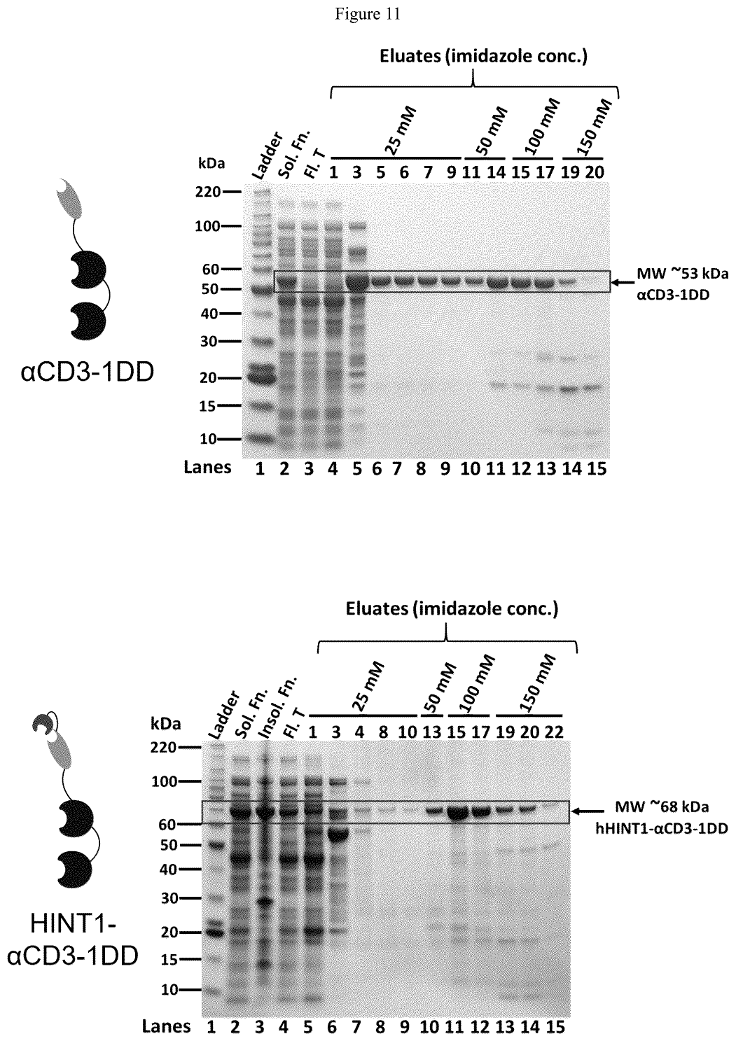

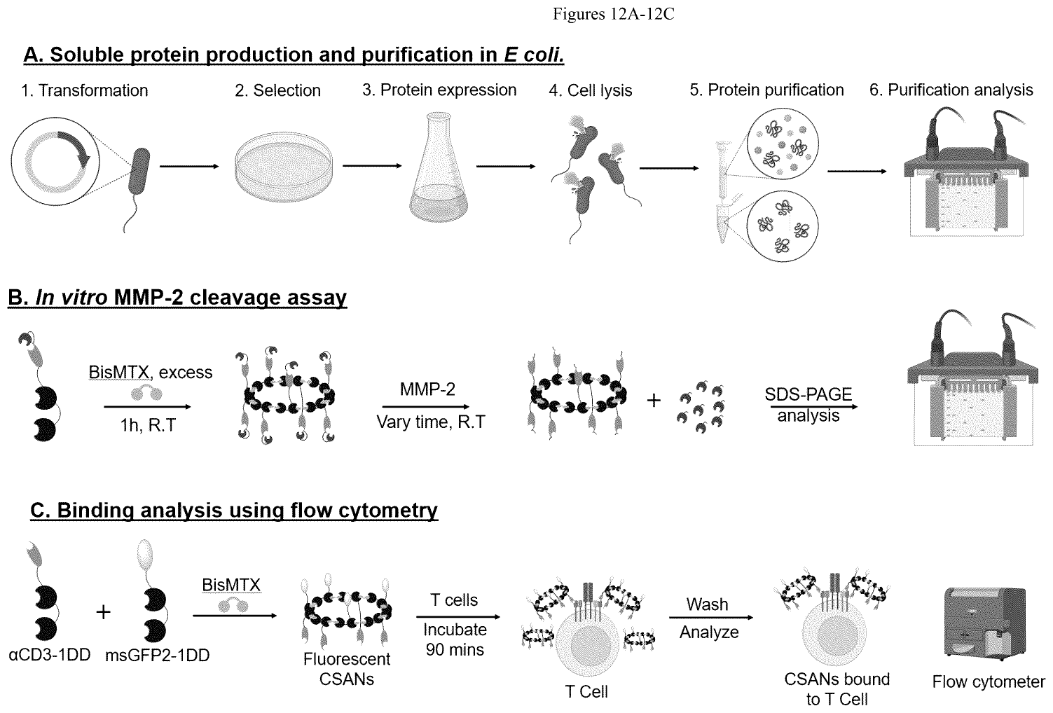

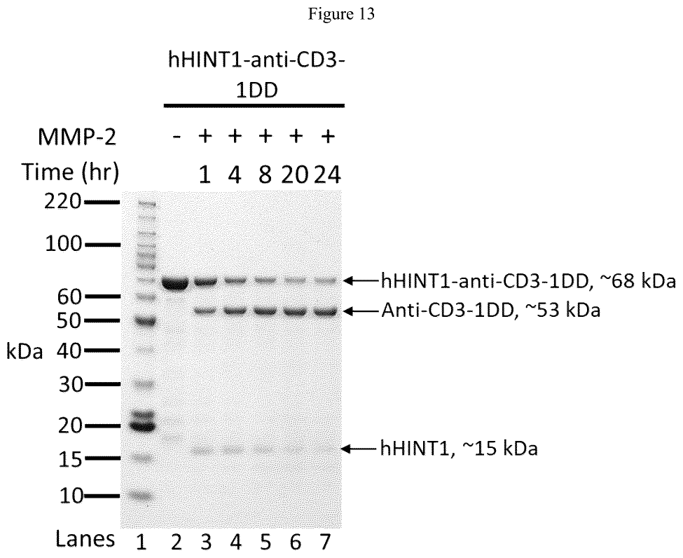

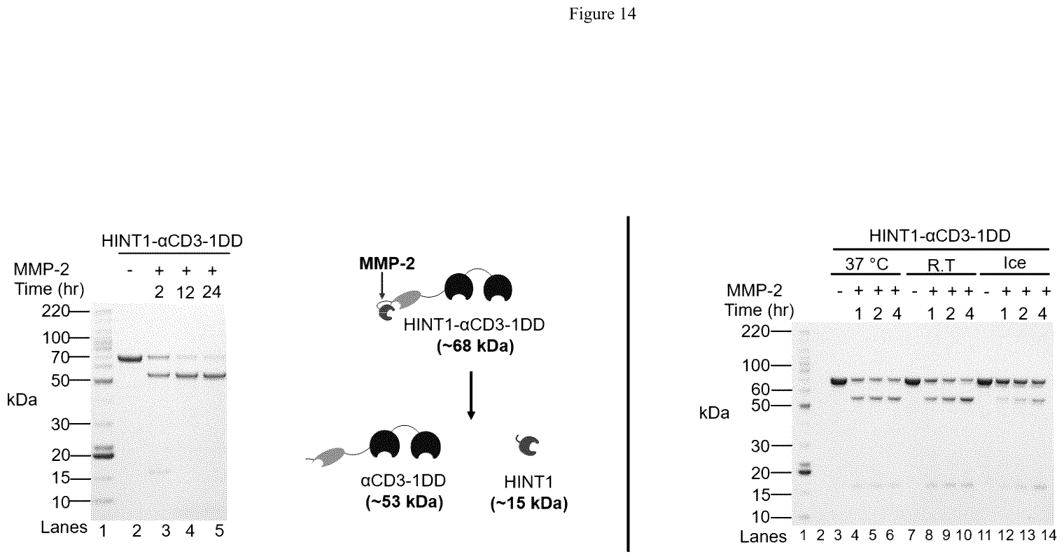

We have successfully expressed and purified genetically masked αCD3-1DD nanobody-based proteins (FIGS. 5-6, FIG. 11). Preliminary studies indicated that both the steric and the affinity (peptide) masks are cleaved by recombinant MMP-2 in vitro (FIG. 7, FIG. 13, FIG. 14). Flow cytometry analysis established a proof-of-concept that masked-αCD3-1DD proteins show reduced binding to T cells but regain binding upon cleavage with MMP-2 (FIG. 8). Future studies may include further characterizations of the proteins using dynamic light scattering, mass spectrometry, and cryo-TEM; and studies for assessing the effect of masking on T cell activation in the presence and absence of target cells; 2D and 3D in vitro cytotoxicity assays.

### Methods/Results

### Design of the Masked-αCD3-1DD Fusion Proteins.

A single-domain antibody (sdAb), or nanobody, that binds to the CD3ε receptor on the surface of T cells was found in the literature. To genetically mask the anti-CD3 nanobody, a structural analysis revealed that the antigen binding region is oriented near the N-terminus of the nanobody. Therefore, we decided to modify the nanobody's N-terminus with a steric or an affinity mask (e.g., FIG. 4B).

We utilized a monomeric version of the human histidine nucleotide triad binding protein 1 (hHINT1) as a steric mask. hHINT1 being a human protein is potentially non-immunogenic. Moreover, the monomeric version is catalytically active, so it could be used to study the biodistribution of the proteins by utilizing fluorescent probes that get activated by hHINT1. Secondly, since the αCD3 nanobody recognizes a linear stretch of the amino acids 1-27 of the N-terminal portion of human CD3ε, we employed this peptide epitope to serve as an affinity mask. Both the steric and affinity masks were genetically fused to the nanobody's N-terminus via a 17 amino acid linker possessing an MMP-2 substrate sequence (GPLGVR (SEQ ID NO: 4)).

### Size Exclusion Chromatography (SEC) Analysis

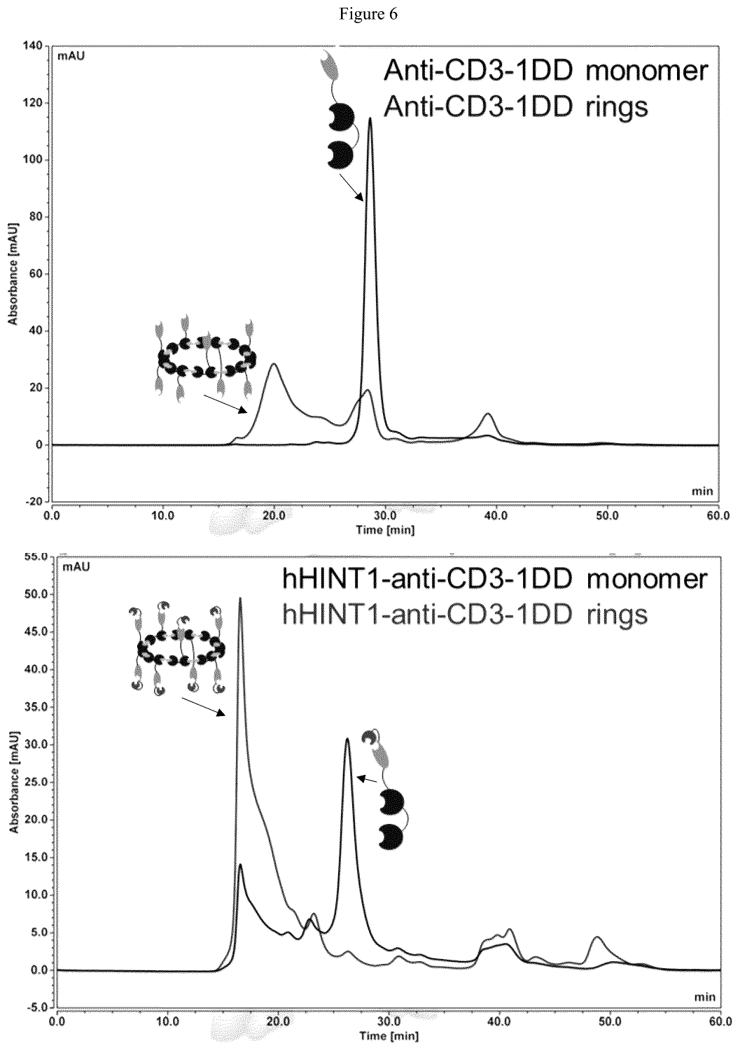

SEC is a technique that separates proteins based on their molecular weight. When the chemical dimerizer, bisMTX is added to a solution of our protein, the monomeric protein should spontaneously self-assemble to form a higher molecular weight species that will show up earlier in a size exclusion chromatogram. The data shown in FIG. 6 indicated that both the anti-CD3-1DD and hHINT1-anti-CD3-1DD proteins could assemble to form nanorings.

### Protease-Cleavage Assay

Since the hHINT1-anti-CD3-1DD protein contains a matrix metalloproteinase-2 (MMP-2) sensitive linker sequence (GLPGVR (SEQ ID NO: 44)), MMP-2 should digest the protein to remove the hHINT1 mask and result in the formation of anti-CD3-1DD. This unmasking or removal of hHINT1 is hypothesized to occur selectively in the tumor tissue due to the upregulated activity of MMP-2.

As seen through the above time-dependent SDS-PAGE analysis (FIG. 7 left panel, FIG. 13 and FIG. 14 left panel), hHINT1-anti-CD3-1DD was almost completely digested by MMP-2 within 24 hours, resulting in the removal of hHINT1 mask and formation of anti-CD3-1DD protein.

Similarly, peptide-anti-CD3-1DD was also digested by MMP-2, resulting in the removal of epitope peptide mask and formation of anti-CD3-1DD protein (FIG. 7 right panel).

### Flow Cytometry Analysis

For proof-of-concept studies, we evaluated the binding potential of the hHINT1-anti-CD3-1DD and anti-CD3-1DD fusion proteins using T cells via flow cytometry. We hypothesized that the hHINT1 mask would occlude the binding of the anti-CD3 nanobody to T cells. Once treated with MMP-2, the hHINT1 mask should be cleaved, and the cleaved protein should bind to T cells.

Flow cytometry was used to evaluate binding of the fusion proteins to surface of T cells. The CD3 targeting masked and unmasked fusion protein were mixed with msGFP2-1DD (monomeric super-folder green fluorescent protein) to form fluorescent protein nanorings. Anti-CD3-1DD fusion protein showed a concentration dependent binding to T cells (FIG. 8A). In contrast, masked fusion proteins hHINT1-anti-CD3-1DD (FIG. 8B) and epitope peptide-anti-CD3-1DD (FIG. 8C) showed little to no binding to T cells as a concentration-dependent shift to the right in the histograms was not observed. hHINT1-anti-CD3-1DD (FIG. 8D) or epitope peptide-anti-CD3-1DD (FIG. 8E) was cleaved with MMP-2 and proteins then regained binding to T cells.

### Dose-Dependent Titration Assay

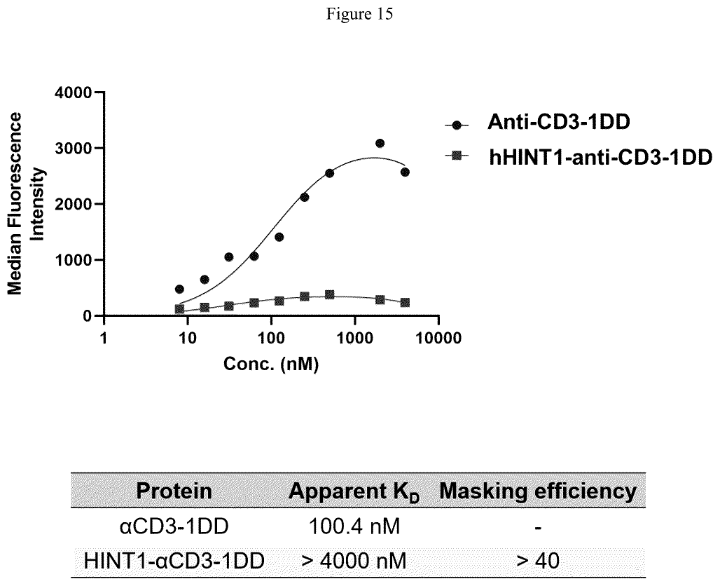

The apparent affinity of the fusion proteins was determined using flow cytometry with T cells isolated from healthy donor blood. Anti-CD3-1DD fusion protein showed an apparent K_{D} value of 100 nM, whereas the hHINT1-anti-CD3-1DD fusion protein showed an apparent K_{D} of greater than 4000 nM (FIG. 15). Therefore, the hHINT1 mask reduced the binding affinity of the anti-CD3 nanobody fusion protein by about 40-fold.

DNA and amino acid sequences of the masked-αCD3-1DD fusion proteins are listed in Table 1 and below:

1. DNA sequence (SEQ ID NO: 26) encoding
hHINT1-αCD3-1DD:
(SEQ ID NO: 26)
ATGGGCCACCATCACCATCATCATATGGCAGATGAGATTG
CCAAGGCTCAGGTCGCTCGGCCTGGTGGCGACACGATCTT
TGGGAAGATCATCCGCAAGGAAATACCAGCCAAAATCATT
TTTGAGGATGACCGGAGCCTTGCTTTCCATGACATTTCCC
CTCAAGCACCAACACATTTTCTGGTGATACCCAAGAAACA
TATATCCCAGATTTCTGTGGCAGAAGATGATGATGAAAGT
CTTCTTGGACACTTAATGATTGTTGGCAAGAAAAGCGCTG
CTGATCTGGGCCTGAATAAGGGTTATCGAATGGAAGTGAA
TGAAGGTTCAGATGGTGGACAGTCTGTCTATCACGTTCAT
CTCCATGTTCTTGGAGGTCGGCAAATGCATTGGCCTCCTG
GTGGAGGTTCAGGTGGTGGACCGCTGGGAGTGCGCGGCAG
CGGAGGCGAGCTCGAGGTGCAGCTGCTCGAGGAGGTGCAG
CTGGTGGAGTCTGGGGGAGGCTTGGTGCAGCCTGGGGGGT
CTCTGAGACTCTCCTGTGCAGCCTCTGGATTCACTTTTGA
TGATTATGGCATGAGCTGGGTCCGACAGGCTCCAGGGAAG
TGGCTGGAGTGGGTCTCAGATATTAGCTGGAATGGTGGTA
GCACATACTATGCAGACTCCGTGAAGGGCCGGTTCACCAT
CTCCAGAGACAACGCCGAGAACACGCTGTATCTGCAAATG
AACAGCCTGAAACCTGACGACACGGCCGTGTATTACTGTG
CAAAAATGGGTGAAGGGGGATGGGGTGCAAATGACTACTG
GGGCCAGGGGACCCAGGTCACCGTCTCCTCCGGAGGCGGA
GCTAGCGGTGGAGGCGGCTCAGGGGGAGGTGGATCCGGAA
TCTCGTTGATTGCGGCATTAGCGGTCGACCGCGTTATCGG
AATGGAAAACGCGATGCCCTGGAATTTACCTGCTGACCTT
GCTTGGTTCAAGCGTAACACTTTAAACAAGCCGGTGATTA
TGGGACGCCACACGTGGGAATCCATCGGCCGCCCTCTGCC
GGGACGTAAGAACATCATTCTTTCAAGCCAACCAGGAACC
GACGATCGCGTGACGTGGGTCAAGAGTGTCGACGAAGCAA
TCGCGGCCGCGGGAGACGTCCCGGAAATCATGGTCATCGG
AGGAGGACGTGTCTATGAGCAGTTTTTGCCTAAGGCGCAG
AAGCTGTACTTAACCCATATCGACGCAGAGGTGGAGGGCG
ACACACACTTCCCCGATTACGAGCCCGATGATTGGGAGTC
AGTGTTCTCAGAATTTCACGACGCGGATGCGCAGAACTCT
CACTCTTATAGTTTCGAGATTTTGGAGCGCCGCGGTGGAA
TTAGTCTTATCGCTGCGTTGGCAGTCGATCGCGTAATCGG
TATGGAGAATGCTATGCCTTGGAACCTTCCCGCAGACTTG
GCCTGGTTCAAACGCAATACTTTAAATAAACCTGTGATTA
TGGGCCGTCATACTTGGGAGTCGATCGGGCGTCCTTTGCC
CGGACGCAAGAATATCATTTTGAGTTCCCAACCGGGCACC
GATGATCGTGTTACGTGGGTTAAGAGTGTGGACGAAGCTA
TCGCCGCTGCTGGGGACGTACCCGAAATTATGGTTATTGG
GGGTGGACGCGTATATGAGCAATTTCTGCCGAAAGCCCAA
AAACTTTATCTTACCCACATTGATGCCGAAGTGGAAGGCG
ATACGCATTTCCCGGACTATGAGCCGGATGATTGGGAATC
AGTGTTTAGCGAGTTTCACGATGCAGACGCTCAGAACAGT
CATTCATACTCGTTTGAGATTTTAGAGCGCCGTGGAGGTG
GCCTTAAGGATTACAAGGACGACGATGACAAGTAA
Protein sequence (SEQ ID NO: 21) of
hHINT1-αCD3-1DD:
(SEQ ID NO: 21)
MGHHHHHHMADEIAKAQVARPGGDTIFGKIIRKEIPAKII
FEDDRSLAFHDISPQAPTHFLVIPKKHISQISVAEDDDES
LLGHLMIVGKKSAADLGLNKGYRMEVNEGSDGGQSVYHVH
LHVLGGROMHWPPGGGSGGGPLGVRGSGGELEVQLLEEVO
LVESGGGLVQPGGSLRLSCAASGFTFDDYGMSWVRQAPGK
WLEWVSDISWNGGSTYYADSVKGRFTISRDNAENTLYLQM
NSLKPDDTAVYYCAKMGEGGWGANDYWGQGTQVTVSSGGG
ASGGGGSGGGGSGISLIAALAVDRVIGMENAMPWNLPADL
AWFKRNTLNKPVIMGRHTWESIGRPLPGRKNIILSSQPGT
DDRVTWVKSVDEAIAAAGDVPEIMVIGGGRVYEQFLPKAQ
KLYLTHIDAEVEGDTHFPDYEPDDWESVFSEFHDADAQNS
HSYSFEILERRGGISLIAALAVDRVIGMENAMPWNLPADL
AWFKRNTLNKPVIMGRHTWESIGRPLPGRKNIILSSQPGT
DDRVTWVKSVDEAIAAAGDVPEIMVIGGGRVYEQFLPKAQ
KLYLTHIDAEVEGDTHFPDYEPDDWESVFSEFHDADAQNS
HSYSFEILERRGGGLKDYKDDDDK
hHINT1 = italics; Gly-Ser linker with MMP-2
cleavage site = bold,
MMP-2 cleavage site = bold, underlined
2. DNA sequence (SEQ ID NO: 30) encoding
Peptide-αCD3-1DD:
(SEQ ID NO: 30)
ATGGGCCACCATCACCATCATCATCAGGATGGCAACGAAG
AAATGGGCGGCATTACCCAGACCCCGTATAAAGTGAGCAT
TAGCGGCACCACCGTGATTCTGACCGGAGGTTCAGGTGGT
GGACCGCTGGGAGTGCGCGGCAGCGGAGGCGAGCTCGAGG
TGCAGCTGCTCGAGGAGGTGCAGCTGGTGGAGTCTGGGGG
AGGCTTGGTGCAGCCTGGGGGGTCTCTGAGACTCTCCTGT
GCAGCCTCTGGATTCACTTTTGATGATTATGGCATGAGCT
GGGTCCGACAGGCTCCAGGGAAGTGGCTGGAGTGGGTCTC
AGATATTAGCTGGAATGGTGGTAGCACATACTATGCAGAC
TCCGTGAAGGGCCGGTTCACCATCTCCAGAGACAACGCCG
AGAACACGCTGTATCTGCAAATGAACAGCCTGAAACCTGA
CGACACGGCCGTGTATTACTGTGCAAAAATGGGTGAAGGG
GGATGGGGTGCAAATGACTACTGGGGCCAGGGGACCCAGG
TCACCGTCTCCTCCGGAGGCGGAGCTAGCGGTGGAGGCGG
CTCAGGGGGAGGTGGATCCGGAATCTCGTTGATTGCGGCA
TTAGCGGTCGACCGCGTTATCGGAATGGAAAACGCGATGC
CCTGGAATTTACCTGCTGACCTTGCTTGGTTCAAGCGTAA
CACTTTAAACAAGCCGGTGATTATGGGACGCCACACGTGG
GAATCCATCGGCCGCCCTCTGCCGGGACGTAAGAACATCA
TTCTTTCAAGCCAACCAGGAACCGACGATCGCGTGACGTG
GGTCAAGAGTGTCGACGAAGCAATCGCGGCCGCGGGAGAC
GTCCCGGAAATCATGGTCATCGGAGGAGGACGTGTCTATG
AGCAGTTTTTGCCTAAGGCGCAGAAGCTGTACTTAACCCA
TATCGACGCAGAGGTGGAGGGCGACACACACTTCCCCGAT
TACGAGCCCGATGATTGGGAGTCAGTGTTCTCAGAATTTC
ACGACGCGGATGCGCAGAACTCTCACTCTTATAGTTTCGA
GATTTTGGAGCGCCGCGGTGGAATTAGTCTTATCGCTGCG
TTGGCAGTCGATCGCGTAATCGGTATGGAGAATGCTATGC
CTTGGAACCTTCCCGCAGACTTGGCCTGGTTCAAACGCAA
TACTTTAAATAAACCTGTGATTATGGGCCGTCATACTTGG
GAGTCGATCGGGCGTCCTTTGCCCGGACGCAAGAATATCA
TTTTGAGTTCCCAACCGGGCACCGATGATCGTGTTACGTG
GGTTAAGAGTGTGGACGAAGCTATCGCCGCTGCTGGGGAC
GTACCCGAAATTATGGTTATTGGGGGTGGACGCGTATATG
AGCAATTTCTGCCGAAAGCCCAAAAACTTTATCTTACCCA
CATTGATGCCGAAGTGGAAGGCGATACGCATTTCCCGGAC
TATGAGCCGGATGATTGGGAATCAGTGTTTAGCGAGTTTC
ACGATGCAGACGCTCAGAACAGTCATTCATACTCGTTTGA
GATTTTAGAGCGCCGTGGAGGTGGCCTTAAGGATTACAAG
GACGACGATGACAAGTAA
Protein sequence (SEQ ID NO: 25) of
peptide-αCD3-1DD:
(SEQ ID NO: 25)
MGHHHHHHQDGNEEMGGITQTPYKVSISGTTVILTGGSGG
GPLGVRGSGGELEVQLLEEVQLVESGGGLVQPGGSLRLSC
AASGFTFDDYGMSWVRQAPGKWLEWVSDISWNGGSTYYAD
SVKGRFTISRDNAENTLYLQMNSLKPDDTAVYYCAKMGEG
GWGANDYWGQGTQVTVSSGGGASGGGGSGGGGSGISLIAA
LAVDRVIGMENAMPWNLPADLAWFKRNTLNKPVIMGRHTW
ESIGRPLPGRKNIILSSQPGTDDRVTWVKSVDEAIAAAGD
VPEIMVIGGGRVYEQFLPKAQKLYLTHIDAEVEGDTHFPD
YEPDDWESVFSEFHDADAQNSHSYSFEILERRGGISLIAA
LAVDRVIGMENAMPWNLPADLAWFKRNTLNKPVIMGRHTW
ESIGRPLPGRKNIILSSQPGTDDRVTWVKSVDEAIAAAGD
VPEIMVIGGGRVYEQFLPKAQKLYLTHIDAEVEGDTHFPD
YEPDDWESVFSEFHDADAQNSHSYSFEILERRGGGLKDYK
DDDDK
Masking peptide = italics;
Gly-Ser linker with MMP-2
cleavage site = bold,
MMP-2 cleavage site = bold, underlined
3. DNA sequence (SEQ ID NO: 27)
encoding αCD3-1DD:
(SEQ ID NO: 27)
ATGGGCCACCATCACCATCATCATGAGGTGCAGCTGCTCG
AGGAGGTGCAGCTGGTGGAGTCTGGGGGAGGCTTGGTGCA
GCCTGGGGGGTCTCTGAGACTCTCCTGTGCAGCCTCTGGA
TTCACTTTTGATGATTATGGCATGAGCTGGGTCCGACAGG
CTCCAGGGAAGTGGCTGGAGTGGGTCTCAGATATTAGCTG
GAATGGTGGTAGCACATACTATGCAGACTCCGTGAAGGGC
CGGTTCACCATCTCCAGAGACAACGCCGAGAACACGCTGT
ATCTGCAAATGAACAGCCTGAAACCTGACGACACGGCCGT
GTATTACTGTGCAAAAATGGGTGAAGGGGGATGGGGTGCA
AATGACTACTGGGGCCAGGGGACCCAGGTCACCGTCTCCT
CCGGAGGCGGAGCTAGCGGTGGAGGCGGCTCAGGGGGAGG
TGGATCCGGAATCTCGTTGATTGCGGCATTAGCGGTCGAC
CGCGTTATCGGAATGGAAAACGCGATGCCCTGGAATTTAC
CTGCTGACCTTGCTTGGTTCAAGCGTAACACTTTAAACAA
GCCGGTGATTATGGGACGCCACACGTGGGAATCCATCGGC
CGCCCTCTGCCGGGACGTAAGAACATCATTCTTTCAAGCC
AACCAGGAACCGACGATCGCGTGACGTGGGTCAAGAGTGT
CGACGAAGCAATCGCGGCCGCGGGAGACGTCCCGGAAATC
ATGGTCATCGGAGGAGGACGTGTCTATGAGCAGTTTTTGC
CTAAGGCGCAGAAGCTGTACTTAACCCATATCGACGCAGA
GGTGGAGGGCGACACACACTTCCCCGATTACGAGCCCGAT
GATTGGGAGTCAGTGTTCTCAGAATTTCACGACGCGGATG
CGCAGAACTCTCACTCTTATAGTTTCGAGATTTTGGAGCG
CCGCGGTGGAATTAGTCTTATCGCTGCGTTGGCAGTCGAT
CGCGTAATCGGTATGGAGAATGCTATGCCTTGGAACCTTC
CCGCAGACTTGGCCTGGTTCAAACGCAATACTTTAAATAA
ACCTGTGATTATGGGCCGTCATACTTGGGAGTCGATCGGG
CGTCCTTTGCCCGGACGCAAGAATATCATTTTGAGTTCCC
AACCGGGCACCGATGATCGTGTTACGTGGGTTAAGAGTGT
GGACGAAGCTATCGCCGCTGCTGGGGACGTACCCGAAATT
ATGGTTATTGGGGGTGGACGCGTATATGAGCAATTTCTGC
CGAAAGCCCAAAAACTTTATCTTACCCACATTGATGCCGA
AGTGGAAGGCGATACGCATTTCCCGGACTATGAGCCGGAT
GATTGGGAATCAGTGTTTAGCGAGTTTCACGATGCAGACG
CTCAGAACAGTCATTCATACTCGTTTGAGATTTTAGAGCG
CCGTGGAGGTGGCCTTAAGGATTACAAGGACGACGATGAC
AAGTAA
Protein sequence (SEQ ID NO: 22)
of αCD3-1DD:
(SEQ ID NO: 22)
MGHHHHHHEVQLLEEVQLVESGGGLVQPGGSLRLSCAASG
FTFDDYGMSWVRQAPGKWLEWVSDISWNGGSTYYADSVKG
RFTISRDNAENTLYLQMNSLKPDDTAVYYCAKMGEGGWGA
NDYWGQGTQVTVSSGGGASGGGGSGGGGSGISLIAALAVD
RVIGMENAMPWNLPADLAWFKRNTLNKPVIMGRHTWESIG
RPLPGRKNIILSSQPGTDDRVTWVKSVDEAIAAAGDVPEI
MVIGGGRVYEQFLPKAQKLYLTHIDAEVEGDTHFPDYEPD
DWESVFSEFHDADAQNSHSYSFEILERRGGISLIAALAVD
RVIGMENAMPWNLPADLAWFKRNTLNKPVIMGRHTWESIG
RPLPGRKNIILSSQPGTDDRVTWVKSVDEAIAAAGDVPEI
MVIGGGRVYEQFLPKAQKLYLTHIDAEVEGDTHFPDYEPD
DWESVFSEFHDADAQNSHSYSFEILERRGGGLKDYKDDDD
K

### Example 2: Masking of a Nanobody Targeting Prostate Specific Membrane Antigen (PSMA) on the Surface of Prostate Cancer Cells

Design of hHINT1-Anti-PSMA-1DD Protein

As described in Example 1, hHINT1 successfully masked the anti-CD3 nanobody. Without wanting to be bound by theory, mechanistically, we believe that the hHINT1 fused to the N-terminus of the anti-CD3 nanobody physically occludes or prevents the binding of the 40 nanobody to CD3. Therefore, since the masking by hHINT1 may be dependent on sterics, hHINT1 should serve as a universal mask that would potentially block the binding of any nanobody to its target of interest. Therefore, to explore the applicability of hHINT1 as a universal mask, we designed an anti-PSMA-1DD and hHINT1-anti-PSMA-1DD protein bearing an anti-PSMA nanobody. A similar design strategy was utilized as described above (protein sequences and DNA enconding hHINT1-anti-PSMA-1DD protein can be found in Table 1; protein sequence for anti-PSMA nanobody can also be found in Table 1, or K chatalic, et al., Journal of Nuclear Medicine July 2015, 56 (7) 1094-1099, the entire content of which is incorporated by reference herein).

### Size Exclusion Chromatography (SEC) Analysis

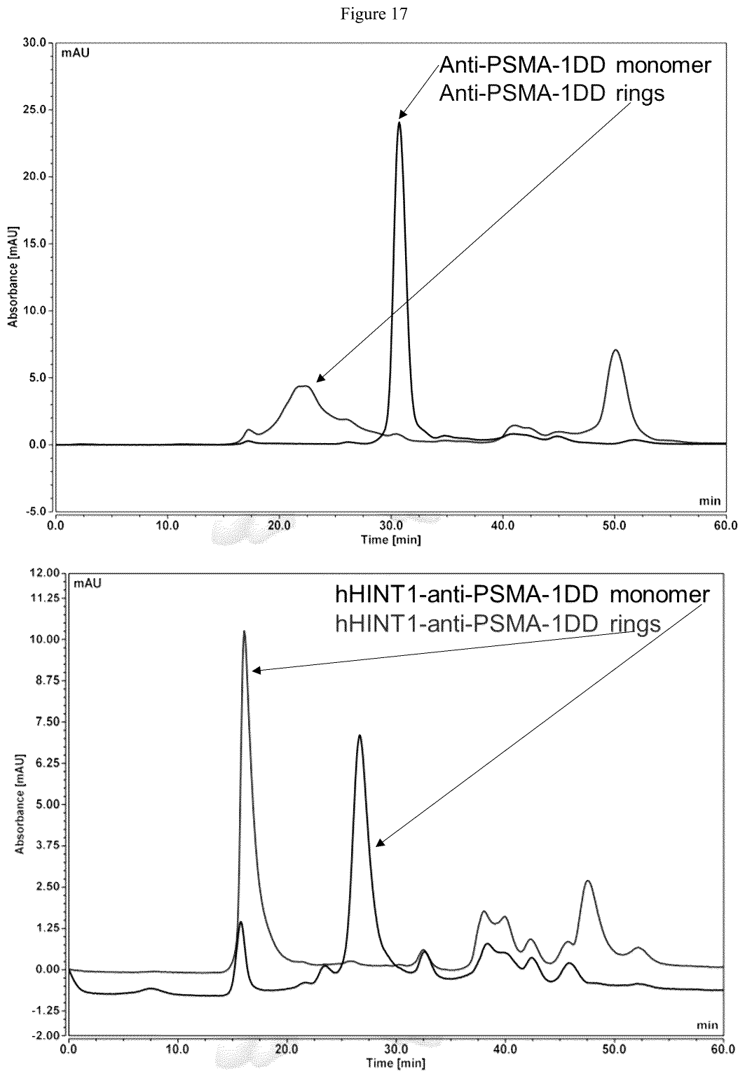

SEC is a technique that separates proteins based on their molecular weight. When the chemical dimerizer, bisMTX is added to a solution of protein, the monomeric protein should spontaneously self-assemble to form a higher molecular weight species that will show up earlier in a size exclusion chromatogram. The data shown in FIG. 17 indicated that both the anti-PSMA-1DD and hHINT1-anti-PSMA-1DD proteins could assemble to form nanorings.

### Clinical Utility

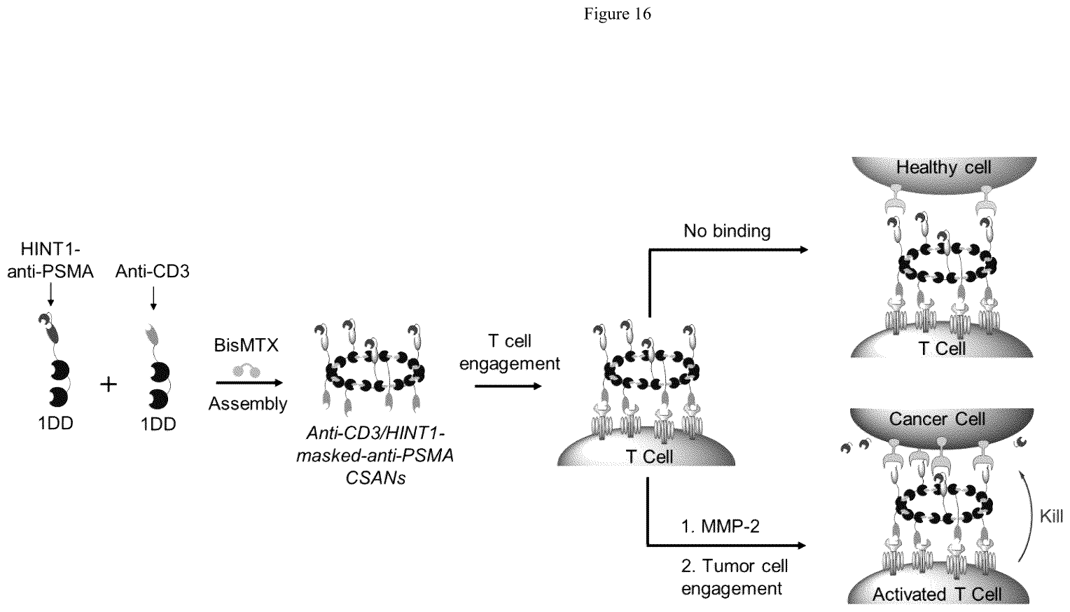

CSANs built using hHINT1-anti-PSMA-1DD and anti-CD3-1DD (anti-CD3 scFv was used herein) fusion proteins will label the surface of T cells. However, since the anti-PSMA nanobody is masked by hHINT1, the nanoring-labeled T cells can not engage PSMA in healthy tissues such as in the salivary gland and the kidney. Once these nanoring-labeled T cells enter the prostate tumor environment, MMP-2 will cleave hHINT1 and the T cells can then subsequently cross-link with tumor cells through the protein nanorings and lead to T cell-mediated cancer cell death (FIG. 16).

### Protease-Cleavage Assay

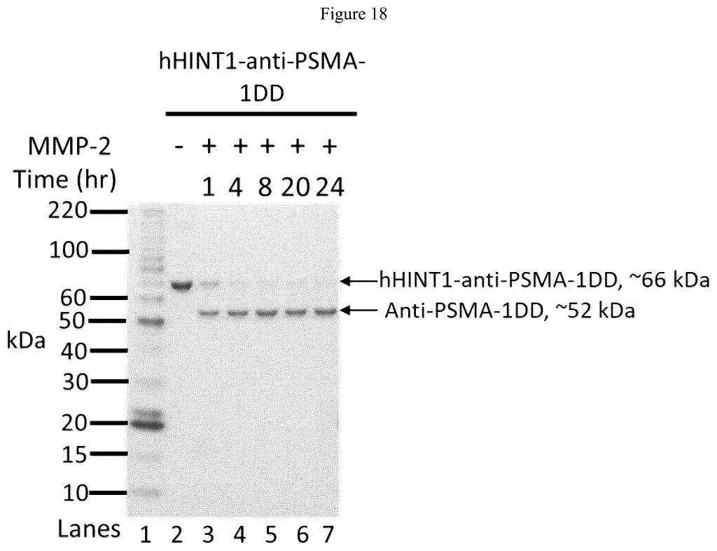

As seen through the above time-dependent SDS-PAGE analysis (FIG. 18), hHINT1-anti-PSMA-1DD was almost completely digested by MMP-2 within 24 hours, resulting in the removal of hHINT1 mask and formation of anti-PSMA-1DD protein.

### In Vitro Cytotoxicity Assay

Anti-PSMA-1DD and anti-CD3 scFv-1DD-based CSANs should redirect T cells to target and destroy PSMA+LNCaP (prostate cancer) cells. Conversely, hHINT1-anti-PSMA-1DD and anti-CD3 scFv-1DD-based CSANs should show a reduction in the lysis of LNCaP cells by T cells as the anti-PSMA nanobody is potentially sterically masked. Anti-CD3 scFv-1DD is also referred to as 1DD-anti-CD3 scFv (see SEQ ID NO:32 in Table 1).

5×10³ LNCaP-Red cells/well were plated in a 96-well flat bottom plate and allowed to adhere overnight. On the day of the assay, appropriate CSAN treatments were made by mixing proteins with bisMTX for 1 hour. T cells were isolated from peripheral blood mononuclear cells (PBMCs) using the Akadeum Human T Cell Isolation Kit. Isolated T cells were resuspended in fresh RPMI media supplemented with 10% FBS. Isolated T cells were diluted appropriately and 5×10³ T cells were added to each well and effector: target cell ratio was kept constant at 1:1. 50 nM CSANs were then added to the plate and the red object sum area/well was measured every 4 hours using a Cytation 10 confocal imager. All treatments were tested in triplicates and data is presented as the mean±SEM.

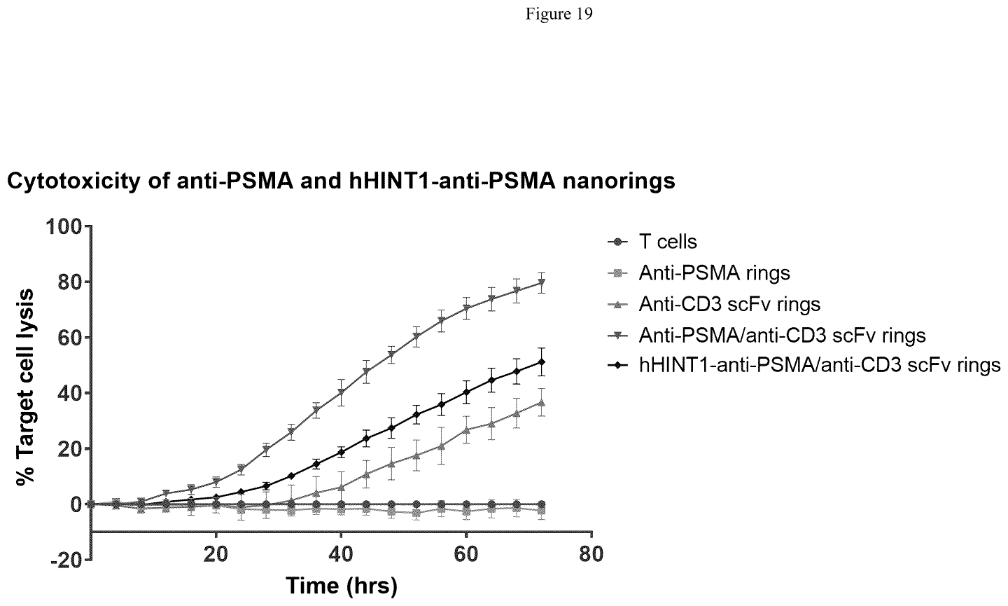

Anti-PSMA/anti-CD3 scFv labeled T cells could kill PSMA+ LNCaP cells. Anti-PSMA/anti-CD3 scFv nanorings efficiently lysed tumor cells over the period of 72 hrs. Anti-CD3 scFv rings (monospecific, do not possess the PSMA-targeting nanobody) showed comparatively lesser tumor cell lysis at 72 hrs. Additionally, the masked protein, hHINT1-anti-PSMA/anti-CD3 scFv showed a reduction in the target cell lysis (approximately 33% reduction) when compared to the anti-PSMA/anti-CD3 scFv treatment (FIG. 19).

This assay further provides a proof-of-concept for the steric masking of the anti-PSMA nanobody by hHINT1. On-going studies are geared to treat the masked protein with MMP-2 to cleave the hHINT1 mask and evaluate the unmasked protein's cytotoxic potential on LNCaP cells.

In summary, data described herein indicated that hHINT1 can sterically mask a nanobody when fused to the nanobody's N-terminus, and that this mask can be removed by MMP-2 that is often upregulated in the tumor microenvironment. Therefore, CSANs bearing hHINT1-masked nanobody fusion proteins have the potential to redirect human T cells specifically to the solid tumors, thereby having minimal on-target, off-tumor toxicity. The hHINT1 mask can potentially be applied as a universal, steric masking strategy to prevent binding of nanobodies to their antigen of interest outside the tumor microenvironment. The hHINT1-masked nanobodies can also be utilized as a cancer imaging tool, as well as for the tumor-specific delivery of anti-cancer drugs.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A recombinant protein, comprising a masking polypeptide linked to a single-domain antibody (sdAb), or an antigen binding fragment thereof, via a protease-sensitive polypeptide linker, wherein the binding affinity of the sdAb, or the antigen binding fragment thereof, to its target antigen is reduced by the masking polypeptide.

2. The recombinant protein of claim 1, wherein the masking polypeptide is linked to the N-terminus of the sdAb, or an antigen binding fragment thereof.

3. The recombinant protein of claim 1, wherein the binding affinity for the target antigen is reduced through steric hindrance provided by the masking polypeptide.

4. The recombinant protein of claim 1, wherein the masking polypeptide comprises an amino acid sequence having at least about 80% sequence identity to SEQ ID NO:3.

5. The recombinant protein of claim 1, wherein the masking polypeptide comprises an epitope polypeptide sequence that specifically binds to the sdAb, or an antigen binding fragment thereof.

6. The recombinant protein of claim 5, wherein the epitope polypeptide sequence comprises a human CD3ε derived polypeptide sequence.

7. The recombinant protein of claim 6, wherein the human CD3ε derived polypeptide sequence comprises an amino acid sequence having at least 90% sequence identity to SEQ ID NO:10.

8. The recombinant protein of claim 1, wherein the protease-sensitive polypeptide linker is capable of being cleaved by a proteolytic enzyme expressed within a solid tumor.

9. The recombinant protein of claim 1, wherein the protease-sensitive polypeptide linker is capable of being cleaved by a matrix metalloproteinase (MMP).

10. The recombinant protein of claim 9, wherein the protease-sensitive polypeptide linker comprises a GPLGVR (SEQ ID NO:4) sequence.

11. The recombinant protein of claim 1, wherein the sdAb is an anti-CD3 sdAb, or an antigen binding fragment thereof.

12. The recombinant protein of claim 1, wherein the sdAb is an anti-tumor associated antigen (anti-TAA) sdAb, or an antigen binding fragment thereof.

13. The recombinant protein of claim 12, wherein the tumor associated antigen (TAA) is selected from the group consisting of EpCAM, PSMA, CD133, and Her2.

14. The recombinant protein of claim 12, wherein the sdAb is an anti-PSMA sdAb, or an antigen binding fragment thereof.

15. The recombinant protein of claim 14, comprising an amino acid sequence having at least 80% sequence identity to SEQ ID NO:8.

16. The recombinant protein of claim 11, comprising an amino acid sequence having at least 80% sequence identity to SEQ ID NO:6 or 11.

17. The recombinant protein of claim 1, further comprising a DHFR domain sequence having at least 80% sequence identity to SEQ ID NO:14.

18. The recombinant protein of claim 17, comprising an amino acid sequence having at least 80% sequence identity to SEQ ID NO: 21, 23, or 25.

19. A polynucleotide comprising a nucleic acid sequence encoding a recombinant protein of claim 1.

20. A chemically self-assembled nanoring (CSAN) comprising a recombinant protein of claim 17.

21. The CSAN of claim 20, wherein the recombinant protein comprises an anti-TAA sdAb, or an antigen binding fragment thereof, and the CSAN further comprises a second recombinant protein comprising an anti-CD3 targeting domain (e.g., anti-CD3 scFv, affibody, Fn3, or sdAb).

22. The CSAN of claim 20, wherein the recombinant protein comprises an anti-CD3 sdAb, or an antigen binding fragment thereof, and the CSAN further comprises a second recombinant protein comprising an anti-TAA targeting domain (e.g., anti-TAA scFv, affibody, Fn3, or sdAb).

23. A pharmaceutical composition comprising a recombinant protein of claim 1 and a pharmaceutically acceptable excipient.

24. A pharmaceutical composition comprising a CSAN of claim 20 and a pharmaceutically acceptable excipient.

25. A method for treating or preventing cancer in an animal, comprising administering a therapeutically effective amount of a recombinant protein of claim 1 to the animal.

26. A method for treating or preventing cancer in an animal, comprising administering a therapeutically effective amount of a CSAN of claim 20 to the animal.
